(19) United States
(12) Patent Application Publication
(10) Pub. No.: US 2010/0120763 A1
(43) Pub. Date: May 13, 2010

(54) IMIDAZO[5,1-C][1,2,4]BENZOTRIAZINE DERIVATIVES AS INHIBITORS OF PHOSPHODIESTERASES

(75) Inventors: Hans Stange, Riesa (DE); Barbara Langen, Radebeul (DE); Ute Egerland, Radebeul (DE); Norbert Hoefgen, Ottendorf-Okrilla (DE); Martina Priebs, Freital (DE); Michael S. Malamas, Jamison, PA (US); James Joseph Erdei, Flourtown, PA (US); Yike Ni, Hillsborough, NJ (US)

Correspondence Address: FISH & RICHARDSON PC, P.O. BOX 1022, MINNEAPOLIS, MN, 55440-1022, US

(73) Assignee: WYETH, Madison, NJ (US)

(21) Appl. No.: 12/614,146

(22) Filed: Nov. 6, 2009

Related U.S. Application Data
(60) Provisional application No. 61/198,695, filed on Nov. 7, 2008.

(51) Int. Cl. A61K 31/5377; C07D 487/04; A61K 31/53

(52) U.S. Cl. 514/233.2; 544/184; 514/243; 544/112

(57) ABSTRACT

The invention relates to imidazo[5,1-c][1,2,4]benzotriazine derivatives of formula I:

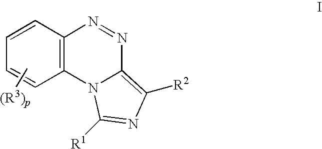

which are inhibitors of phosphodiesterase 2 or 10 useful in treating central nervous system diseases such as psychosis and also in treating, for example, obesity, type 2 diabetes, metabolic syndrome, glucose intolerance, and pain.

This application claims the benefit of priority of U.S. Provisional Application No. 61/198,695, which is incorporated herein by reference in its entirety.

### TECHNICAL FIELD

The invention relates to imidazo[5,1-c][1,2,4]benzotriazine derivatives which are inhibitors of phosphodiesterase 2 or 10, useful in treating central nervous system diseases such as psychosis and also in treating, for example, obesity, type 2 diabetes, metabolic syndrome, glucose intolerance, and pain.

### BACKGROUND

Psychotic disorders, especially schizophrenia, are severe mental disorders which extremely impair daily life. The symptoms of psychosis may be divided into two fractions. In the acute phase, it is predominated by hallucinations and delusions being called the positive symptoms. When the agitated phase abates the so called negative symptoms become obvious. They include cognitive deficits, social phobia, reduced vigilance, indifference and deficits in verbal learning and memory, verbal fluency and motor function.

Although several antipsychotics have become available, the present therapy of psychosis is not satisfactory. The classic antipsychotics, such as haloperidol, with a high affinity to dopamine D2 receptor show extreme side effects, such as extrapyramidal symptoms (=EPS) and do not improve the negative symptoms of schizophrenia so that they do not enable the patient to return to everyday life. Other antipsychotics, such as clozapine, can show negative side effects, such as agranulocytosis.

In addition to psychotic disorders, depression is a severe mental disorder which extremely impairs daily life. Its prevalence is about 10% of the world population with an incidence of 2% according to WHO. Women are more affected than men and elder people more than younger people. The disorder mostly implies a life-long treatment due to the progress of the disease and permanent total disability.

The most prominent symptoms of the disease are anhedonia, feeling of hopelessness, decreased self esteem, loss of appetite and sleep disturbance. Most patients are suicidal. Depression is often combined with anxiety disorders. Interestingly, it is less known that depression is also regularly associated with various cognitive impairments (Gualtieri et al., 2006; Mandelli et al., 2006). Here, deficits of attentional and executive function are mostly reported (Paelecke-Habermann et al., 2005). Cognitive deficits are even discussed to be involved in the development of the disease (Beck depression model, Beck, 2008) Actually, the severity of the cognitive deficits may predict non-response to certain antidepressant treatment (Dunkin et al., 2000; Gorlyn et al., 2008).

Elder antidepressants are reported to impair memory in animal models of learning and memory probably due to their anticholinergic component (Kumar and Kulkarni, 1996). In contrast, SSRIs, especially fluoxetine, are described to impair hippocampal-independent but not hippocampal dependent learning in different rodent models (Valluzi and Chan, 2007). Some modern antidepressants are described to reverse cognitive impairments associated with stress-induced depression in rats (Ramanathan et al., 2003).

At least, in clinic current therapy it is not possible to fully reverse cognitive deficits. Thus, in depressive patients who had been successfully treated cognitive performance could be improved but not normalised (Gualtieri et al., 2006). Therefore, an antidepressant with higher efficacy on cognitive impairment may improve disease outcome.

Phosphodiesterases (PDE) are expressed in nearly all mammalian cells. As a consequence, they play an important role in numerous physiological and pathophysiological processes. To date eleven families of phosphodiesterases have been identified in mammals (Essayan, 2001). It is well established that PDEs are critically involved in cell signalling. Specifically, PDEs are known to inactivate the cyclic nucleotides cAMP and/or cGMP (Soderling and Beavo, 2000). The cyclic nucleotides cAMP and cGMP are synthesised by the adenylyl and guanylyl cyclases and are second messengers that control many key cellular functions. The synthesis of cAMP and cGMP is regulated by different G-protein-coupled receptor types including dopamine D1 and D2 receptors. By its effect PDEs may reduce or even eliminate the signal cascade initiated by activating extracellular receptors. PDE inhibitors, in contrast, may prolong or amplify this effect. Thereby the different phosphodiesterase families and their inhibitors may very specifically participate in the maintenance and the regulation of the homeostasis of an organism.

The phosphodiesterases of the different families vary in their substrate selectivity. Thus, some families only hydrolyse cAMP others only cGMP. Some phosphodiesterases inactivate both cAMP and cGMP (Menniti et al., 2006). Furthermore, there is a difference in the distribution of the different phosphodiesterases within the organism and additionally, within any particular tissue or organ. For instance, the distribution pattern of the phosphodiesterases within the brain is quite specific (Menniti et al., 2006).

Finally, phosphodiesterase families have different regulatory properties and intracellular location; some are bound to cell membranes and some are dissociated in the cytoplasm, additionally, a division into various intracellular compartments has been reported (Conti and Jin, 1999).

These differences in the function and location of the different PDE enzyme families suggests that the individual phosphodiesterases are selectively involved in regulating many different physiological processes. Accordingly, selective phosphodiesterase inhibitors may with fine specificity regulate different physiological and pathophysiological processes.

PDE2 hydrolyses both, cGMP and cAMP and is activated by cGMP (Menniti et al., 2006). It is abundantly expressed in the brain (Bolger et al., 1994). Here, PDE2 mRNA is mainly distributed in olfactory bulb, olfactory tubercle, cortex, amygdala, striatum, and hippocampus (Lakics et al., 2005; van Staveren et al., 2003).

The expression of PDE2 in the hippocampus and the cortex indicate an involvement in the mechanism of learning and memory. This is supported by the fact that increased levels of both cGMP and cAMP are involved in the process of LTP forming (Blokland et al., 2006; Prickaerts et al., 2002). LTP is regarded as the electrophysiological basis of long term memory (Baddeley, 2003). Boess et al. (2004) showed that PDE2 inhibitors amplify the generation of long term potentiation (LTP). Additionally, it is reported that the selective PDE2 inhibitor BAY60-7550 enhances learning and memory in rats and mice in different animal models (Boess et al., 2004; Rutten et al., 2006). Thus, BAY60-7550 is efficacious in the novel object recognition test, the social recognition test and the T-maze, an animal model of working memory.

Furthermore, the expression of PDE2 in the nucleus accumbens (part of the striatum), the olfactory bulb, the olfactory tubercle and the amygdale supports additional involvement of PDE2 in the pathophysiology of anxiety and depression (Modell et al., 1990). As described above, PDE2 inhibitors increase cAMP and cGMP in neuronal cells. There is evidence that chronic administration of antidepressants up-regulates the cAMP pathway at several levels, including increased expression of the cAMP response element binding protein (CREB) (Duman, 1998; Nibuya et al., 1996). In contrast, patients with depression show an impairment of the cAMP pathway. Thus, Shelton et al. (1999) detected a reduction of cAMP associated protein kinase A in depressive patients. Finally, Masood et al. (2008) report an anxiolytic effect of PDE2 inhibition in mice. They reversed oxidative stress-induced anxiety by the PDE2 inhibitor BAY60-7550.

Consequently, PDE2 inhibitors are described to have a potential to alleviate central nervous system (CNS) disorders, e.g. depression and Alzheimer's disease but also peripheral diseases like metabolic disorders, septic shock and cancer. PDE10 (PDE10A) is primarily expressed in the brain and here in the nucleus accumbens and the caudate putamen. Areas with moderate expression are the thalamus, hippocampus, frontal cortex and olfactory tubercle (Menniti et al., William Harvey Research Conference, Porto, Dec. 6-8, 2001). All these brain areas are described to participate in the pathomechanism of schizophrenia (Lapiz et al., Neurosci Behav Physiol 33: 13-29, 2003) so that the location of the enzyme indicates a predominate role in the pathomechanism of psychosis.

PDE2 inhibitors address a novel target in the brain. PDE2 inhibitors are described to have an antidepressant and anxiolytic effect. Additionally, they improve impaired but also un-impaired learning and memory (Boess et al., 2004; Rutten et al., 2006b). Thus, PDE2 inhibitors are a promising new target to improve the therapy of CNS disorders, especially depression and Alzheimer's disease.

Several families of PDE2 inhibitors are known. Imidazotriazinones are claimed in WO 02068423 for the treatment of e.g. memory deficiency, cognitive disorders, dementia and Alzheimer's disease. Oxindoles are described in WO 05041957 for the treatment of dementia. Further inhibitors of PDE2 are known from WO 07121319 for the treatment of anxiety and depression, from WO 06072615, WO 06072612, WO 06024640 and WO 05113517 for the treatment of arthritis, cancer, edema and septic shock, from WO 05063723 for the treatment of renal and liver failure, liver dysfunction, restless leg syndrome, rheumatic disorders, arthritis, rhinitis, asthma and obesity, from WO 05041957 for the treatment of cancer and thrombotic disorders, from WO 06102728 for the treatment of angina pectoris and hypertension from WO 08043461 for the treatment of cardiovascular disorders, erectile dysfunction, inflammation and renal failure and from WO 05061497 for the treatment of e.g. dementia, memory disorders, cancer and osteoporosis.

Finally, benzodiazepines are claimed in WO 2005063723 for the general treatment of CNS diseases including anxiety, depression, ADHD, neurodegeneration, Alzheimer's disease and psychosis.

Unfortunately, there is no PDE2 inhibitor that could be successfully developed to become a treatment medication. Most of them are not optimal for CNS penetration or suffer on pure physical properties.

There is still an urgent need to provide new PDE2 inhibitors with improved properties for the treatment of diseases where inhibition of PDE2 is of therapeutic value.

In the striatum PDE10A is predominately found in the medium spiny neurons and they are primarily associated to the postsynaptic membranes of these neurons (Xie et al., Neuroscience 139: 597-607, 2006). By this location PDE10A may have an important influence on the signal cascade induced by dopaminergic and glutamatergic input on the medium spiny neurons two neurotransmitter systems playing a predominate role in the pathomechanism of psychosis.

Phosphodiesterase (PDE) 10A, in particular, hydrolyses both cAMP and cGMP having a higher affinity for cAMP (Km=0.05 μM) than for cGMP (Km=3 μM) (Soderling et al., Curr. Opin. Cell Biol 12: 174-179, 1999).

Psychotic patients have been shown to have a dysfunction of cGMP and cAMP levels and its downstream substrates (Kaiya, Prostaglandins Leukot Essent Fatty Acids 46: 33-38, 1992; Muly, Psychopharmacol Bull 36: 92-105, 2002; Garver et al., Life Sci 31: 1987-1992, 1982). Additionally, haloperidol treatment has been associated with increased cAMP and cGMP levels in rats and patients, respectively (Leveque et al., J Neurosci 20: 4011-4020, 2000; Gattaz et al., Biol Psychiatry 19: 1229-1235, 1984). As PDE10A hydrolyses both cAMP and cGMP (Kotera et al., Biochem Biophys Res Commun 261: 551-557, 1999), an inhibition of PDE10A would also induce an increase of cAMP and cGMP and thereby have a similar effect on cyclic nucleotide levels as haloperidol.

The antipsychotic potential of PDE10A inhibitors is further supported by studies of Kostowski et al. (Pharmacol Biochem Behav 5: 15-17, 1976) who showed that papaverine, a moderate selective PDE10A inhibitor, reduces apomorphine-induced stereotypies in rats, an animal model of psychosis, and increases haloperidol-induced catalepsy in rats while concurrently reducing dopamine concentration in rat brain, activities that are also seen with classical antipsychotics. This is further supported by a patent application establishing papaverine as a PDE10A inhibitor for the treatment of psychosis (US Patent Application Pub. No. 2003/0032579).

In addition to classical antipsychotics which mainly ameliorate the positive symptoms of psychosis, PDE10A also bears the potential to improve the negative and cognitive symptoms of psychosis.

Focusing on the dopaminergic input on the medium spiny neurons, PDE10A inhibitors by up-regulating cAMP and cGMP levels act as D1 agonists and D2 antagonists because the activation of Gs-protein coupled dopamine D1 receptor increases intracellular cAMP, whereas the activation of the Gi-protein coupled dopamine D2 receptor decreases intracellular cAMP levels through inhibition of adenylyl cyclase activity (Mutschler et al., Mutschler Arzneimittelwirkungen. 8th ed. Stuttgart: Wissenschaftliche Verlagsgesellschaft mbH, 2001).

Elevated intracellular cAMP levels mediated by D1 receptor signalling seems to modulate a series of neuronal processes responsible for working memory in the prefrontal cortex (Sawaguchi, Parkinsonism Relat Disord 7: 9-19, 2000), and it is reported that D1 receptor activation may improve working memory deficits in schizophrenic patients (Castner et al., Science 287: 2020-2022, 2000).

Further indication of an effect of PDE10A inhibition on negative symptoms of psychosis was given by Rodefer et al. (Eur. J Neurosci 21: 1070-1076, 2005) who could show that papaverine reverses attentional set-shifting deficits induced by subchronic administration of phencyclidine, an NMDA antagonist, in rats. Attentional deficits including an impairment of shifting attention to novel stimuli belongs to the negative symptoms of schizophrenia. In the study the attentional deficits were induced by administering phencyclidine for 7 days followed by a washout period. The PDE10A inhibitor papaverine was able to reverse the enduring deficits induced by the subchronic treatment.

Several routes are described for the synthesis of 1,2,4-Benzotriazines. Sadchikova et al. published an intermolecular cyclisation of 2-hydroxyphenyl- or 2-methoxyphenylazo dyes (Sadchikova et al. 2000 and 2005). An other opportunity is the reaction of 1-(2-aminophenyl)-imidazoles with sodium nitrite and H2SO4. In this case the resulting triazine was observed as a side product in low yields (6%) only (Antonini et al. 1976; Simonov et al. 1969 and 1967). In US 2006/0111568 a number of 1,2,4-benzotriazines are claimed to be azo dyes. These derivatives are substituted with cyano groups at the imidazole ring and with hydroxyl groups at the benzo ring of the molecule. Salts of similar derivatives were claimed in DE 2348382. In this case the benzo ring needed to be substituted with an amino group. In WO 2005/014595 the use of benzo-1,2,4-triazines as a herbicide or plant growth regulator is claimed.

In conclusion, there is a need for new antipsychotic and antidepressant agents. This invention addresses this need and others.

### SUMMARY

The present invention provides, inter alia, compounds of formula (I):

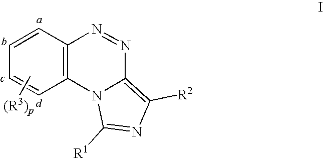

or pharmaceutically acceptable salts thereof.

The present invention also provides a pharmaceutical composition comprising a compound of formula (I), or a pharmaceutically acceptable salt thereof, and a pharmaceutically acceptable carrier.

The present invention further provides a method of treating disorders associated with phosphodiesterase 2 or 10 hyperactivity, the method comprising administering to a patient in need thereof a therapeutically effective amount of a compound of formula (I), or a pharmaceutically acceptable salt thereof.

The present invention also provides a method of treating a central nervous system disorder in a patient in need thereof comprising, administering to said patient a therapeutically effective amount of a compound of formula (I), or a pharmaceutically acceptable salt thereof.

The present invention further provides a method of treating obesity, type II diabetes, metabolic syndrome, glucose intolerance and related health risks, symptoms or disorders in a patient in need thereof comprising administering to said patient a therapeutically effective amount of a compound of formula (I), or a pharmaceutically acceptable salt thereof.

The present invention also provides a method of treating or preventing disorders associated with enhanced endothelial activity, impaired endothelial barrier or enhanced neoangiogenesis, septic shock; vascular edema, reduced natriuria pathology, inflammatory diseases, asthma, rhinitis, arthritis, rheumatoid diseases, autoimmune diseases, acute renal or liver failure, liver dysfunction, and benign or malignant neoplasia in a patient in need thereof comprising, administering to said patient a therapeutically effective amount of a compound of formula (I), or a pharmaceutically acceptable salt thereof.

The present invention further provides a method of treating or preventing a disorder associated with thrombosis or embolism in a patient in need thereof comprising, administering to said patient a therapeutically effective amount of a compound of formula (I), or a pharmaceutically acceptable salt thereof.

The present invention still further provides a method of treating pain or a pain disorder selected from inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain in a patient in need thereof, comprising administering to said patient a compound of formula (I), or a pharmaceutically acceptable salt thereof.

The present invention also provides a compound for use in any of the methods described herein. The present invention further provides use of a compound for the preparation of a medicament for use in any of the methods described herein.

The details of one or more embodiments of the invention are set forth in the accompanying drawings and the description below. Other features, objects, and advantages of the invention will be apparent from the description and drawings, and from the claims.

### DETAILED DESCRIPTION

The present invention provides, inter alia, a compound of formula (I):

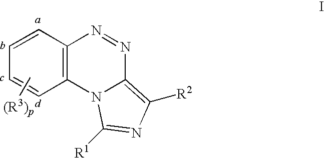

or a pharmaceutically acceptable salt thereof wherein:

a, b, c, and d indicate four possible positions on the ring for each R³, when present;

p is 0 or an integer from 1 to 4;

R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;

R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;

each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;

any two groups R³ may together be alkylene or alkenylene completing a 3- to 8-membered saturated or unsaturated ring together with the carbon atoms to which they are attached, which ring is unsubstituted or substituted with one or more independently selected Z groups; or

any two groups of R³ may, together with the atoms to which they are attached, form a heterocyclo group which is unsubstituted or substituted with one or more independently selected Z groups;

each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;

each R⁵, R⁶, R⁷, R⁸, R⁹ and R¹⁰ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or

R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or

any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;

each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;

each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more Z¹ groups;

each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;

each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;

each Y³ and Y⁴ is independently selected from a single bond, alkylene, alkenylene, and alkynylene; and

each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy.

In some embodiments, the following provisos apply:
(a) when p is 0, then R¹ and R² are not each H or each methyl;
(b) when R¹ is H; R² is —C(O)O-(ethyl); p is 1; and R³ is at the c position of the ring; then R³ is other than hydroxyl;
(c) when R¹ is H; R² is nitro or —C(O)O-(ethyl); p is 2; and each R³ is at the a and c positions of the ring; then each R³ is other than methoxy;
(d) when R¹ is H; p is 2; each R³ is methyl; and the two R³ groups are at the a and c positions of the ring; then R² is other than —C(O)OH, —C(O)O-(ethyl), or benzylthio;
(e) when R¹ is H; R² is nitro, —C(O)O-(ethyl), —C(O)NH₂, or —CONH-(methyl); p is 1; and R³ is at the b position of the ring, then R³ is other than methyl;
(f) when R¹ is H; R² is —C(O)O-(ethyl); p is 1; and R³ is at the b position of the ring; then R³ is other than amino;
(g) when R¹ is methyl, 1,3-dixolan-2-yl, hydroxymethyl, or formyl; and R² is H, then p is other than 0;
(h) when R¹ is H; R² is H; p is 1; and R³ is at the c position of the ring; then R³ is other than chloro or dimethylamino;
(i) when R¹ is H; p is 1; R³ is hydroxyl; and R³ is at the c position of the ring; then R² other than cyano;
(j) when R² is H; p is 1; R³ is hydroxyl; and R³ is at the c position of the ring; then R¹ other than cyano;
(k) when R¹ is H; R² is H; p is 1; and R³ is at the b position of the ring; then R³ is other than chloro, bromo, methyl, methoxy, nitro, and trifluoromethyl;
(l) when R² is H, p is 1; R³ is methoxy; and R³ is at the b position of the ring; then R¹ is other than methyl, ethyl, methoxy, ethoxy, trifluoromethyl or phenyl;
(m) when R¹ is H, p is 1; R³ is methoxy; and R³ is at the b position of the ring; then R² is other than methyl, ethyl, methoxy, ethoxy, trifluoromethyl or phenyl;
(n) when R² is H, p is 0 or 1; R³ is chloro (when p is 1); and R³ is at the b position of the ring (when p is 1); then R¹ is other than chloro, cyano, nitro, methyl, ethyl, isopropyl, methoxy, ethoxy, trifluoromethyl, phenyl, methylthio, —C(O)O-(methyl), —C(O)-(methyl), —C(O)N-(methyl)₂, —N(methyl)₂, or benzyl;
(o) when R¹ is H, p is 0 or 1; R³ is chloro (when p is 1); and R³ is at the b position of the ring (when p is 1); then R² is other than chloro, cyano, nitro, methyl, ethyl, isopropyl, methoxy, ethoxy, trifluoromethyl, phenyl, methylthio, —C(O)O-(methyl), —C(O)O-(ethyl), —C(O)-(methyl), —C(O)N-(methyl)₂, —N(methyl)₂, or benzyl;
(p) when R¹ is H; p is 2; each R³ is chloro; and the two R³ groups are at the b and d positions of the ring; then R² is other than H;
(q) when two R³ groups, together with the atoms to which they are attached, form an unsubstituted benzene ring, and R¹ is H, then R² is other than hydrogen, nitro, —C(O)O-(ethyl), —C(O)NHNH₂, —C(O)NH₂, —C(O)NH-(methyl), —C(O)NH-(phenyl), —C(O)NH-(cyclohexyl), —C(O)NH-(morpholin-1-yl), —C(O)NH-(piperidin-1-yl), —C(O)NH-(4-methylphenyl), —C(O)NH-(4-chlorophenyl), and —NH—C(O)O-(ethyl); and
(r) when two R³ groups, together with the atoms to which they are attached, form an heterocyclo ring, that heterocyclo ring is other than a 12-membered ring with 4 or more oxygen atoms.

In some embodiments, p is 1, 2, or 3; R¹ and R² are other than H; and each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂.

In some embodiments, p is 1, 2, or 3. In some embodiments, p is 1 or 2. In some embodiments, p is 1. In some embodiments, p is 2.

In some embodiments, R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups.

In some embodiments, R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups; and wherein each R⁵, R⁶, R⁸, R⁹, and R¹⁰ is independently selected from H, alkyl, and haloalkyl.

In some embodiments, R¹ is selected from halo, alkyl, cycloalkyl, aryl, and heterocyclo, wherein said alkyl, cycloalkyl, aryl, and heterocyclo are each unsubstituted or substituted with one or more independently selected Z groups.

In some embodiments, R¹ is selected from alkyl, cycloalkyl, aryl, and heterocyclo, wherein said alkyl, cycloalkyl, aryl, and heterocyclo are each unsubstituted or substituted with one or more independently selected Z groups.

In some embodiments, R¹ is selected from alkyl, wherein said alkyl is unsubstituted or substituted with one or more independently selected Z groups.

In some embodiments, R¹ is selected from cycloalkyl, wherein said cycloalkyl is unsubstituted or substituted with one or more independently selected Z groups.

In some embodiments, R¹ is selected from aryl and heteroaryl, wherein said aryl and heteroaryl are each unsubstituted or substituted with one or more independently selected Z groups.

In some embodiments, R¹ is heterocyclo, which is unsubstituted or substituted with one or more independently selected Z groups.

In some embodiments, R¹ is heteroaryl, which is unsubstituted or substituted with one or more independently selected Z groups.

In some embodiments, R¹ is aryl, which is unsubstituted or substituted with one or more independently selected Z groups.

In some embodiments, R¹ is selected from bromo, ethyl, propyl, isobutyl, cyclohexyl, a phenyl ring, a thiophene ring, a pyrazole ring, an isooxazole ring, a thiazole ring, and a pyridine ring; wherein said ethyl, propyl, isobutyl, cyclohexyl, a phenyl ring, a thiophene ring, a pyrazole ring, an isooxazole ring, a thiazole ring, and a pyridine ring are each unsubstituted or substituted with one or more independently selected Z groups.

In some embodiments, R¹ is selected from H, bromo, ethyl, propyl, isobutyl, cyclohexyl, phenyl, thiophen-2-yl, 1H-pyrazol-5-yl, 1H-pyrazol-4-yl, furan-3-yl, isooxazol-4-yl, thiazol-5-yl, pyridin-2-yl, pyridin-3-yl, and pyridin-4-yl; wherein said ethyl, propyl, isobutyl, cyclohexyl, phenyl, thiophen-2-yl, 1H-pyrazol-5-yl, 1H-pyrazol-4-yl, isooxazol-4-yl, thiazol-5-yl, pyridin-2-yl, pyridin-3-yl, and pyridin-4-yl are each unsubstituted or substituted with one or more independently selected Z groups.

In some embodiments, R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups.

In some embodiments, R² is alkyl. In some embodiments, R² is methyl.

In some embodiments, each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups.

In some embodiments, each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups; and wherein each R⁵, R⁶, R⁸, R⁹, and R¹⁰ is independently selected from hydrogen, alkyl, and haloalkyl.

In some embodiments, each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups.

In some embodiments, each R³ is independently selected from halo, alkyl, —OH, —OR⁴, aryl, and heterocyclo, wherein said alkyl, aryl, and heterocyclo are each unsubstituted or substituted with one or more independently selected Z groups; and wherein each R⁴ is independently alkyl, haloalkyl, cycloalkylalkyl or aralkyl.

In some embodiments, each R³ is independently selected from chloro, fluoro, —OH, trifluoromethyl, methoxy, difluoromethoxy, cyclopropylmethoxy, piperidinyl, morpholinyl, piperazinyl, phenyl, 1H-imidazol-1-yl, and benzyloxy; wherein said piperidinyl, morpholinyl, piperazinyl, phenyl, and 1H-imidazol-1-yl are each unsubstituted or substituted with one or more independently selected Z groups.

In some embodiments, each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein said alkyl, cycloalkyl, cycloalkylalkyl, aryl, aralkyl, heterocyclo, and heterocycloalkyl is unsubstituted or substituted with one or more independently selected Z¹ groups.

In some embodiments, each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein each R¹¹ is independently alkyl or haloalkyl; and wherein each R¹², R¹³, R¹⁴, R¹⁵, R¹⁶, and R¹⁷ is independently selected from hydrogen, alkyl, and haloalkyl.

In some embodiments, each Z is independently selected from halo, cyano, nitro, alkyl, haloalkyl, cycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein each R¹¹ is independently alkyl or haloalkyl; and wherein each R¹², R¹³, R¹⁴, R¹⁵, R¹⁶, and R¹⁷ is independently selected from H, alkyl, and haloalkyl.

In some embodiments, each Z is independently selected from halo, cyano, nitro, alkyl, haloalkyl, alkoxy, haloalkoxy, cycloalkyl, —OH, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo; wherein each R¹¹ is independently alkyl or haloalkyl; and wherein each R¹², R¹³, and R¹⁷ is independently selected from H and alkyl.

In some embodiments, each Z group is independently selected from halo, alkyl, haloalkyl, alkoxy, haloalkoxy, and —C(O)NR¹²R¹³.

In some embodiments, each Z group is independently selected from chloro, fluoro, methyl, isopropyl, trifluoromethyl, methoxy, ethoxy, isoproxy, n-propoxy, butoxy, trifluoromethoxy, and —C(O)NH₂.

In some embodiments, each Z is independently selected from halo, alkyl, haloalkyl, alkoxy, and cycloalkyl.

In some embodiments, each Z is independently selected from chloro, fluoro, methyl, isobutyl, trifluoromethyl, ethoxy, propoxy, butoxy, and cyclohexyl.

In some embodiments:

p is 1, 2, or 3;

R¹ is selected from alkyl, aryl, aralkyl or heterocyclo, unsubstituted or substituted with one to three independently selected Z groups;

R² is selected from alkyl; and

each R³ is independently selected from —OH, —OR⁴, halo, cyano, nitro and —NR⁵R⁶, wherein Y¹ represents a single bond.

In some embodiments:

p is 1, 2, or 3;

R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and) —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups;

R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups;

each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups; and

each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein said alkyl, cycloalkyl, cycloalkylalkyl, aryl, aralkyl, heterocyclo, and heterocycloalkyl is unsubstituted or substituted with one or more independently selected Z¹ groups.

In some embodiments:

p is 1, 2, or 3;

R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups;

R² is selected from alkyl;

each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups;

each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein said alkyl, cycloalkyl, cycloalkylalkyl, aryl, aralkyl, heterocyclo, and heterocycloalkyl is unsubstituted or substituted with one or more independently selected Z¹ groups;

each R¹¹ is independently alkyl or haloalkyl; and

each R⁵, R⁶, R⁸, R⁹, R¹⁰, R¹², R¹³, R¹⁴, R¹⁵, R¹⁶, and R¹⁷ is independently selected from H, alkyl, and haloalkyl.

In some embodiments:

p is 1, 2, or 3;

R¹ is selected from halo, alkyl, cycloalkyl, aryl, and heteroaryl, wherein said alkyl, cycloalkyl, aryl, and heteroaryl are each unsubstituted or substituted with one or more independently selected Z groups;

R² is selected from alkyl;

each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;

each Z is independently selected from halo, cyano, nitro, alkyl, haloalkyl, alkoxy, haloalkoxy, cycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo;

each R¹¹ is independently alkyl or haloalkyl; and

each R¹², R¹³, R¹⁴, R¹⁵, R¹⁶, and R¹⁷ is independently selected from H, alkyl, and haloalkyl.

In some embodiments:

p is 1, 2, or 3;

R¹ is selected from alkyl, cycloalkyl, aryl, and heteroaryl, wherein said alkyl, cycloalkyl, aryl, and heteroaryl are each unsubstituted or substituted with one or more independently selected Z groups;

R² is selected from alkyl;

each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;

each Z is independently selected from halo, cyano, nitro, alkyl, haloalkyl, alkoxy, haloalkoxy, cycloalkyl, —OH, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo;

each R¹¹ is independently alkyl or haloalkyl; and

each R¹², R¹³, and R¹⁷ is independently selected from H, alkyl, and haloalkyl.

In some embodiments:

p is 1, 2, or 3;

R¹ is alkyl or cycloalkyl, which are each unsubstituted or substituted with one or more independently selected Z groups;

R² is selected from alkyl;

each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;

each Z is independently selected from halo, cyano, nitro, alkyl, haloalkyl, alkoxy, haloalkoxy, cycloalkyl, —OH, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo;

each R¹¹ is independently alkyl or haloalkyl; and

each R¹², R¹³, and R¹⁷ is independently selected from H, alkyl, and haloalkyl.

In some embodiments:

p is 1, 2, or 3;

R¹ is aryl or heteroaryl, which are each unsubstituted or substituted with one or more independently selected Z groups;

R² is selected from alkyl;

each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;

each Z is independently selected from halo, cyano, nitro, alkyl, haloalkyl, alkoxy, haloalkoxy, cycloalkyl, —OH, —OR¹¹, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo;

each R¹¹ is independently alkyl or haloalkyl; and

each R¹², R¹³, and R¹⁷ is independently selected from H, alkyl, and haloalkyl.

In some embodiments:

p is 1, 2, or 3;

R¹ is selected from H, bromo, ethyl, propyl, isobutyl, cyclohexyl, a phenyl ring, a thiophene ring, a pyrazole ring, an isooxazole ring, a thiazole ring, and a pyridine ring; wherein said ethyl, propyl, isobutyl, cyclohexyl, a phenyl ring, a thiophene ring, a pyrazole ring, an isooxazole ring, a thiazole ring, and a pyridine ring are each unsubstituted or substituted with one or more Z groups independently selected from halo, alkyl, haloalkyl, alkoxy, haloalkoxy, and —C(O)NR¹²R¹³.

R² is selected from alkyl;

each R³ is independently selected from halo, alkyl, —OH, —OR⁴, aryl, and heterocyclo, wherein said alkyl, aryl, and heterocyclo are each unsubstituted or substituted with one or more Z groups independently selected from halo, alkyl, haloalkyl, alkoxy, and cycloalkyl;

each R¹² and R¹³ is independently selected from H and alkyl.

In some embodiments:

p is 1, 2, or 3;

R¹ is selected from H, bromo, ethyl, propyl, isobutyl, cyclohexyl, phenyl, thiophen-2-yl, 1H-pyrazol-5-yl, 1H-pyrazol-4-yl, isooxazol-4-yl, thiazol-5-yl, pyridin-2-yl, pyridin-3-yl, and pyridin-4-yl; wherein said ethyl, propyl, isobutyl, cyclohexyl, phenyl, thiophen-2-yl, 1H-pyrazol-5-yl, 1H-pyrazol-4-yl, isooxazol-4-yl, thiazol-5-yl, pyridin-2-yl, pyridin-3-yl, and pyridin-4-yl are each unsubstituted or substituted with one or more groups independently selected from chloro, fluoro, methyl, isopropyl, trifluoromethyl, methoxy, ethoxy, isoproxy, n-propoxy, butoxy, trifluoromethoxy, and —C(O)NH₂;

R² is methyl; and

each R³ is independently selected from chloro, fluoro, —OH, trifluoromethyl, methoxy, difluoromethoxy, cyclopropylmethoxy, piperidinyl, morpholinyl, piperazinyl, phenyl, 1H-imidazol-1-yl, and benzyloxy; wherein said piperidinyl, morpholinyl, piperazinyl, phenyl, and 1H-imidazol-1-yl are are each unsubstituted or substituted with one or more groups independently selected from chloro, fluoro, methyl, isobutyl, trifluoromethyl, ethoxy, propoxy, butoxy, and cyclohexyl.

In some embodiments, the compound is a compound of Formula Ib:

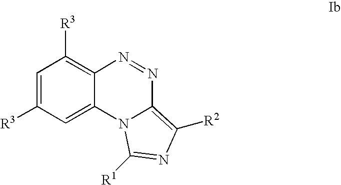

or a pharmaceutically acceptable salt thereof.

In some embodiments, the compound is a compound of Formula Ic:

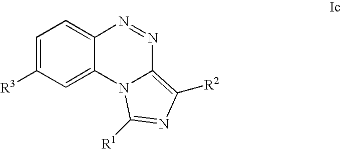

or a pharmaceutically acceptable salt thereof.

The present invention further provides a compound of formula (Ia):

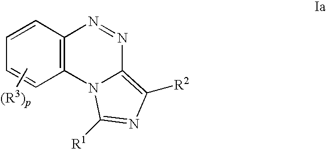

wherein:

p is 0 or an integer from 1 to 4,

each R¹, and R², and R³, are independently selected from:

hydrogen or R⁴, where R⁴ is alkyl, alkenyl, alkynyl, cycloalkyl, cycloalkylalkyl, cycloalkenyl, cycloalkenylalkyl, aryl, aralkyl, heterocyclo, or heterocycloalkyl, each of which is unsubstituted or substituted with one or more (preferably, one to three) groups Z;

—OH or —OR⁴;

—SH or —SR⁴;

—C(O)_{q}H, —C(O)_{q}R⁴, or —O—C(O)_{q}R⁴, where q is 1 or 2;

—SO₃H or —S(O)R⁴

halo;

cyano;

nitro;

—Y¹—NR⁵R⁶;

—Y¹—N(R⁷)—Y²—NR⁸R⁹;

—Y¹—N(R¹⁰)—Y²—R⁴;

—P(O)(OR⁴)₂;

any two groups R³ may together be alkylene or alkenylene completing a 3- to 8-membered saturated or unsaturated ring together with the carbon atoms to which they are attached, which ring is unsubstituted or substituted with one or more groups Z; or

any two groups of R³ may, together with the atoms to which they are attached, form a heterocyclo group which is unsubstituted or substituted with one or more groups Z;

R⁵, R⁶, R⁷, R⁸, R⁹ and R¹⁰, are independently hydrogen or R⁴;

R⁵ and R⁶ may together be alkylene or alkenylene, completing a 3- to 8-membered saturated or unsaturated ring with the nitrogen atom to which they are attached, which ring is unsubstituted or substituted with one or more groups Z;

any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more groups Z;

Z groups are each independently:

hydrogen or R¹¹; where R¹¹ is alkyl, alkenyl, alkynyl, cycloalkyl, cycloalkylalkyl, cycloalkenyl, cycloalkenylalkyl, aryl, aralkyl, heterocyclo, or heterocycloalkyl, each of which is unsubstituted or substituted with one or more (preferably, one to three) groups Z¹;

—OH or —OR¹¹;

—SH or —SR¹¹;

—C(O)_{q}H, —C(O)_{q}R¹¹, or —O—C(O)_{q}R¹¹, where q is 1 or 2;

—SO₃H or —S(O)R¹¹

halo;

cyano;

nitro;

—Y¹—NR⁵R⁶;

Y¹—N(R⁷)—Y²—NR⁸R⁹;

Y¹—N(R¹⁰)—Y²—R¹¹;

oxo

—O—C(O)—R¹¹;

Y¹ and Y² are each independently:

a single bond;

—Y³—S(O)_{q}—Y⁴—;

—Y³—C(O)—Y⁴—;

—Y³—C(S)—Y⁴—;

Y³—O—Y⁴—;

—Y³—S—Y⁴—;

—Y³—O—C(O)—Y⁴—; or

Y³—C(O)—O—Y⁴—;

Y³ and Y⁴ are each independently:

a single bond;

alkylene;

alkenylene; or

alkynylene.

In some embodiments, p is selected from 1, 2 or 3.

In some embodiments, R¹ is selected from alkyl, aryl, aralkyl or heterocyclo, unsubstituted or substituted with one to three groups Z.

In some embodiments, R² is selected from hydrogen or alkyl.

In some embodiments, each R³ is independently selected from hydrogen, —OH, —OR⁴, halo, cyano, nitro or —Y¹—NR⁵R⁶ (wherein Y¹ represents a single bond).

In some embodiments, the compound is selected from:
8-fluoro-3-methyl-1-propyl-imidazo[5,1-c]-1,2,4-benzotriazine;
1-ethyl-8-fluoro-3-methyl-imidazo[5,1-c]-1,2,4-benzotriazine;
1-cyclohexyl-8-fluoro-3-methyl-imidazo[5,1-c]-1,2,4-benzotriazine;
1-(2,5-dichlorophenyl)-8-methoxy-3-methyl-imidazo[5,1-c]-1,2,4-benzotriazine;
1-(2,5-dichlorophenyl)-7-fluoro-3-methyl-imidazo[5,1-c]-1,2,4-benzotriazine; and
1-(2,5-dichlorophenyl)-7-methoxy-3-methyl-imidazo[5,1-c]-1,2,4-benzotriazine;

or a pharmaceutically acceptable salt thereof.

In some embodiments, the compound is selected from:
8-fluoro-3-methyl-1-propyl-imidazo[5,1-c][1,2,4]benzotriazine;
7-methoxy-3-methyl-1-propyl-imidazo[5,1-c][1,2,4]benzotriazine;
1-ethyl-8-fluoro-3-methyl-imidazo[5,1-c][1,2,4]benzotriazine;
1-cyclohexyl-8-fluoro-3-methyl-imidazo[5,1-c][1,2,4]benzotriazine;
1-(2,5-dichlorophenyl)-8-methoxy-3-methyl-imidazo[5,1-c][1,2,4]benzotriazine;
1-(2,5-dichlorophenyl)-7-fluoro-3-methyl-imidazo[5,1-c][1,2,4]benzotriazine; and
1-(2,5-dichlorophenyl)-7-methoxy-3-methyl-imidazo[5,1-c][1,2,4]benzotriazine;

or a pharmaceutically acceptable salt thereof.

In some embodiments, the compound is selected from:
1-(2,5-dichlorophenyl)-8-fluoro-3-methylbenzo[e]imidazo[5,1-c][1,2,4]triazine;
8-fluoro-1-isobutyl-3-methylbenzo[e]imidazo[5,1-c][1,2,4]triazine;
1-sec-butyl-8-fluoro-3-methylbenzo[e]imidazo[5,1-c][1,2,4]triazine;
8-fluoro-3-methylbenzo[e]imidazo[5,1-c][1,2,4]triazine;
8-fluoro-3-methyl-1-(2-methylpyridin-3-yl)benzo[e]imidazo[5,1-c][1,2,4]triazine;
1-(2-chlorophenyl)-8-fluoro-3-methylbenzo[e]imidazo[5,1-c][1,2,4]triazine;
1-(2,3-dichlorophenyl)-8-fluoro-3-methylbenzo[e]imidazo[5,1-c][1,2,4]triazine;
8-fluoro-3-methyl-1-(1-methyl-1H-pyrazol-5-yl)benzo[e]imidazo[5,1-c][1,2,4]triazine;
8-fluoro-1-(2-methoxyphenyl)-3-methylbenzo[e]imidazo[5,1-c][1,2,4]triazine;
8-fluoro-3-methyl-1-o-tolylbenzo[e]imidazo[5,1-c][1,2,4]triazine;
1-(2-chloro-5-methoxyphenyl)-8-fluoro-3-methylbenzo[e]imidazo[5,1-c][1,2,4]triazine;
1-(4-chloropyridin-3-yl)-8-fluoro-3-methylbenzo[e]imidazo[5,1-c][1,2,4]triazine;
8-fluoro-1-(2-fluoro-5-isopropoxyphenyl)-3-methylbenzo[e]imidazo[5,1-c][1,2,4]triazine;
8-fluoro-1-(2-fluoro-5-(trifluoromethyl)phenyl)-3-methylbenzo[e]imidazo[5,1-c][1,2,4]triazine;
1-(5-butoxy-2-fluorophenyl)-8-fluoro-3-methylbenzo[e]imidazo[5,1-c][1,2,4]triazine;
8-fluoro-1-(2-fluoro-5-propoxyphenyl)-3-methylbenzo[e]imidazo[5,1-c][1,2,4]triazine;
1-(5-ethoxy-2-fluorophenyl)-8-fluoro-3-methylbenzo[e]imidazo[5,1-c][1,2,4]triazine;
7-fluoro-3-methyl-1-(2-methylpyridin-3-yl)benzo[e]imidazo[5,1-c][1,2,4]triazine;
7-fluoro-1-(2-methoxyphenyl)-3-methylbenzo[e]imidazo[5,1-c][1,2,4]triazine;
7-fluoro-1-(4-fluoro-2-methylphenyl)-3-methylbenzo[e]imidazo[5,1-c][1,2,4]triazine;
7-fluoro-3-methyl-1-o-tolylbenzo[e]imidazo[5,1-c][1,2,4]triazine;
1-(2-chloro-5-methoxyphenyl)-7-fluoro-3-methylbenzo[e]imidazo[5,1-c][1,2,4]triazine;
7-fluoro-1-(2-fluoro-5-(trifluoromethyl)phenyl)-3-methylbenzo[e]imidazo[5,1-c][1,2,4]triazine;
7-fluoro-1-(2-fluoro-5-isopropoxyphenyl)-3-methylbenzo[e]imidazo[5,1-c][1,2,4]triazine;
1-(5-butoxy-2-fluorophenyl)-7-fluoro-3-methylbenzo[e]imidazo[5,1-c][1,2,4]triazine;
7-fluoro-1-(2-fluoro-5-propoxyphenyl)-3-methylbenzo[e]imidazo[5,1-c][1,2,4]triazine;
1-(2-chlorophenyl)-7-methoxy-3-methylbenzo[e]imidazo[5,1-c][1,2,4]triazine;
7-methoxy-3-methylbenzo[e]imidazo[5,1-c][1,2,4]triazine;
7-methoxy-3-methyl-1-(2-methylpyridin-3-yl)benzo[e]imidazo[5,1-c][1,2,4]triazine;
8-chloro-1-(2,5-dichlorophenyl)-3-methylbenzo[e]imidazo[5,1-c][1,2,4]triazine;
8-chloro-3-methyl-1-(2-methylpyridin-3-yl)benzo[e]imidazo[5,1-c][1,2,4]triazine;
8-chloro-1-(2-methoxyphenyl)-3-methylbenzo[e]imidazo[5,1-c][1,2,4]triazine;
8-chloro-3-methyl-1-(1-methyl-1H-pyrazol-5-yl)benzo[e]imidazo[5,1-c][1,2,4]triazine;
2-(8-chloro-3-methylbenzo[e]imidazo[5,1-c][1,2,4]triazin-1-yl)benzamide;
8-chloro-3-methylbenzo[e]imidazo[5,1-c][1,2,4]triazine;
8-chloro-1-(2-fluoro-5-isopropoxyphenyl)-3-methylbenzo[e]imidazo[5,1-c][1,2,4]triazine;
1-(2-fluoro-5-isopropoxyphenyl)-8-methoxy-3-methylbenzo[e]imidazo[5,1-c][1,2,4]triazine;
6,8-dimethoxy-3-methyl-1-(4-methylpyridin-3-yl)imidazo[5,1-c][1,2,4]benzotriazine;
1-(2-chlorophenyl)-7,8-dimethoxy-3-methylbenzo[e]imidazo[5,1-c][1,2,4]triazine;
6,8-dimethoxy-3-methyl-1-(3-methylpyridin-4-yl)imidazo[5,1-c][1,2,4]benzotriazine;
6,8-dimethoxy-3-methyl-1-(2-methylpyridin-3-yl)imidazo[5,1-c][1,2,4]benzotriazine;
7,8-dimethoxy-3-methyl-1-o-tolylbenzo[e]imidazo[5,1-c][1,2,4]triazine;
7,8-dimethoxy-3-methyl-1-(pyridin-2-yl)benzo[e]imidazo[5,1-c][1,2,4]triazine;
1-(3,5-dimethyl-1H-pyrazol-4-yl)-6,8-dimethoxy-3-methylimidazo[5,1-c][1,2,4]benzotriazine;
6,8-dimethoxy-3-methyl-1-(1,3,5-trimethyl-1H-pyrazol-4-yl)imidazo[5,1-c][1,2,4]benzotriazine;
1-isobutyl-7,8-dimethoxy-3-methylbenzo[e]imidazo[5,1-c][1,2,4]triazine;
1-bromo-7,8-dimethoxy-3-methylbenzo[e]imidazo[5,1-c][1,2,4]triazine;
1-(2,5-dichlorophenyl)-7,8-dimethoxy-3-methylbenzo[e]imidazo[5,1-c][1,2,4]triazine;
1-(2-chloro-5-methylphenyl)-7,8-dimethoxy-3-methylbenzo[e]imidazo[5,1-c][1,2,4]triazine;
7,8-dimethoxy-3-methyl-1-(2-(trifluoromethyl)phenyl)benzo[e]imidazo[5,1-c][1,2,4]triazine;
1-(2-chlorophenyl)-7,8-difluoro-3-methylbenzo[e]imidazo[5,1-c][1,2,4]triazine;
7,8-difluoro-1-isobutyl-3-methylbenzo[e]imidazo[5,1-c][1,2,4]triazine;
6-methoxy-3-methyl-1-(2-methylphenyl)imidazo[5,1-c][1,2,4]benzotriazine;
1-(2-chlorophenyl)-6-methoxy-3-methylbenzo[e]imidazo[5,1-c][1,2,4]triazine;
6-methoxy-3-methyl-1-(3-methylpyridin-4-yl)imidazo[5,1-c][1,2,4]benzotriazine;
6-methoxy-3-methyl-1-(3-methylpyridin-4-yl)-8-morpholin-4-ylimidazo[5,1-c][1,2,4]benzotriazine;
6-methoxy-3-methyl-1-(2-methylpyridin-3-yl)-8-morpholin-4-ylimidazo[5,1-c][1,2,4]benzotriazine
1-(2-chlorophenyl)-6-methoxy-3-methyl-8-morpholin-4-ylimidazo[5,1-c][1,2,4]benzotriazine;
6-methoxy-3-methyl-1-(2-methylphenyl)-8-morpholin-4-ylimidazo[5,1-c][1,2,4]benzotriazine;
6-fluoro-8-methoxy-1-(3-methoxyphenyl)-3-methylimidazo[5,1-c][1,2,4]benzotriazine;
1-(5-chloro-2-methoxyphenyl)-6-fluoro-8-methoxy-3-methylimidazo[5,1-c][1,2,4]benzotriazine;
6-fluoro-1-(4-fluoro-2-methylphenyl)-8-methoxy-3-methylimidazo[5,1-c][1,2,4]benzotriazine;
1-(2-chloro-4-fluorophenyl)-6-fluoro-8-methoxy-3-methylimidazo[5,1-c][1,2,4]benzotriazine;
1-(2-chloro-4-methylphenyl)-6-fluoro-8-methoxy-3-methylimidazo[5,1-c][1,2,4]benzotriazine;
1-(2-chloro-5-methylphenyl)-6-fluoro-8-methoxy-3-methylimidazo[5,1-c][1,2,4]benzotriazine;
6-fluoro-1-(5-fluoro-2-methylphenyl)-8-methoxy-3-methylimidazo[5,1-c][1,2,4]benzotriazine;
6-fluoro-1-(2-fluoro-4-methylphenyl)-8-methoxy-3-methylimidazo[5,1-c][1,2,4]benzotriazine;
6-fluoro-1-(2-fluoro-5-methylphenyl)-8-methoxy-3-methylimidazo[5,1-c][1,2,4]benzotriazine;
6-fluoro-1-(2-fluoro-5-methoxyphenyl)-8-methoxy-3-methylimidazo[5,1-c][1,2,4]benzotriazine;
1-(2-chloro-5-ethoxyphenyl)-6-fluoro-8-methoxy-3-methylimidazo[5,1-c][1,2,4]benzotriazine;
1-(2-chloro-5-methoxyphenyl)-6-fluoro-8-methoxy-3-methylimidazo[5,1-c][1,2,4]benzotriazine;
1-(5-chloro-2-methylphenyl)-6-fluoro-8-methoxy-3-methylimidazo[5,1-c][1,2,4]benzotriazine;
1-(2-chloro-5-fluorophenyl)-6-fluoro-8-methoxy-3-methylimidazo[5,1-c][1,2,4]benzotriazine;
6-fluoro-8-methoxy-3-methyl-1-(3-methylthiophen-2-yl)imidazo[5,1-c][1,2,4]benzotriazine;
1-[2-chloro-5-(trifluoromethyl)phenyl]-6-fluoro-8-methoxy-3-methylimidazo[5,1-c][1,2,4]benzotriazine;
1-[2-chloro-5-(trifluoromethoxy)phenyl]-6-fluoro-8-methoxy-3-methylimidazo[5,1-c][1,2,4]benzotriazine;
6-fluoro-1-(3-fluoro-2-methylphenyl)-8-methoxy-3-methylimidazo[5,1-c][1,2,4]benzotriazine;
6-fluoro-8-methoxy-3-methyl-1-(1,3,5-trimethyl-1H-pyrazol-4-yl)imidazo[5,1-c][1,2,4]benzotriazine;
6-fluoro-8-methoxy-1-(2-methoxyphenyl)-3-methylimidazo[5,1-c][1,2,4]benzotriazine;
6-fluoro-8-methoxy-3-methyl-1-pyridin-4-ylimidazo[5,1-c][1,2,4]benzotriazine;
1-(5-chloro-2-fluorophenyl)-6-fluoro-8-methoxy-3-methylimidazo[5,1-c][1,2,4]benzotriazine;
1-(3,5-dimethylisoxazol-4-yl)-6-fluoro-8-methoxy-3-methylimidazo[5,1-c][1,2,4]benzotriazine;
6-fluoro-8-methoxy-3-methyl-1-pyridin-3-ylimidazo[5,1-c][1,2,4]benzotriazine;
1-(2,4-dimethyl-1,3-thiazol-5-yl)-6-fluoro-8-methoxy-3-methylimidazo[5,1-c][1,2,4]benzotriazine;
6-fluoro-1-(6-fluoro-5-methylpyridin-3-yl)-8-methoxy-3-methylimidazo[5,1-c][1,2,4]benzotriazine;
6-fluoro-1-(6-fluoro-2-methylpyridin-3-yl)-8-methoxy-3-methylimidazo[5,1-c][1,2,4]benzotriazine;
6-fluoro-1-(2-fluoropyridin-3-yl)-8-methoxy-3-methylimidazo[5,1-c][1,2,4]benzotriazine;
6-fluoro-8-methoxy-1-(5-methoxypyridin-3-yl)-3-methylimidazo[5,1-c][1,2,4]benzotriazine;
1-(3,5-dimethyl-1H-pyrazol-4-yl)-6-fluoro-8-methoxy-3-methylimidazo[5,1-c][1,2,4]benzotriazine;
6-fluoro-8-methoxy-3-methyl-1-(4-methylpyridin-3-yl)imidazo[5,1-c][1,2,4]benzotriazine;
4-fluoro-3-(6-fluoro-8-methoxy-3-methylimidazo[5,1-c][1,2,4]benzotriazin-1-yl)benzamide;
8-fluoro-6-methoxy-1-(3-methoxyphenyl)-3-methylimidazo[5,1-c][1,2,4]benzotriazine;
8-fluoro-6-methoxy-1-(2-methoxyphenyl)-3-methylimidazo[5,1-c][1,2,4]benzotriazine;
1-[2-chloro-5-(trifluoromethyl)phenyl]-8-fluoro-6-methoxy-3-methylimidazo[5,1-c][1,2,4]benzotriazine;
1-(5-chloro-2-methoxyphenyl)-8-fluoro-6-methoxy-3-methylimidazo[5,1-c][1,2,4]benzotriazine;
8-fluoro-1-(5-fluoro-2-methylphenyl)-6-methoxy-3-methylimidazo[5,1-c][1,2,4]benzotriazine;
1-(5-chloro-2-methylphenyl)-8-fluoro-6-methoxy-3-methylimidazo[5,1-c][1,2,4]benzotriazine;
8-fluoro-1-(2-fluoro-5-methylphenyl)-6-methoxy-3-methylimidazo[5,1-c][1,2,4]benzotriazine;
1-(5-chloro-2-fluorophenyl)-8-fluoro-6-methoxy-3-methylimidazo[5,1-c][1,2,4]benzotriazine;
1-(2-chloro-5-methylphenyl)-8-fluoro-6-methoxy-3-methylimidazo[5,1-c][1,2,4]benzotriazine;
1-(2-chloro-5-ethoxyphenyl)-8-fluoro-6-methoxy-3-methylimidazo[5,1-c][1,2,4]benzotriazine;
8-fluoro-6-methoxy-3-methyl-1-pyridin-3-ylimidazo[5,1-c][1,2,4]benzotriazine;
8-fluoro-1-(4-fluoro-2-methylphenyl)-6-methoxy-3-methylimidazo[5,1-c][1,2,4]benzotriazine;
1-(2-chloro-4-fluorophenyl)-8-fluoro-6-methoxy-3-methylimidazo[5,1-c][1,2,4]benzotriazine;
1-(2-chloro-4-methylphenyl)-8-fluoro-6-methoxy-3-methylimidazo[5,1-c][1,2,4]benzotriazine;
8-fluoro-1-(2-fluoro-5-methoxyphenyl)-6-methoxy-3-methylimidazo[5,1-c][1,2,4]benzotriazine;
8-fluoro-1-(2-fluoro-4-methylphenyl)-6-methoxy-3-methylimidazo[5,1-c][1,2,4]benzotriazine;
1-(3,5-dimethylisoxazol-4-yl)-8-fluoro-6-methoxy-3-methylimidazo[5,1-c][1,2,4]benzotriazine;
8-fluoro-6-methoxy-3-methyl-1-(1,3,5-trimethyl-1H-pyrazol-4-yl)imidazo[5,1-c][1,2,4]benzotriazine;
8-fluoro-1-(3-fluoro-2-methylphenyl)-6-methoxy-3-methylimidazo[5,1-c][1,2,4]benzotriazine;
1-(2-chloro-5-fluorophenyl)-8-fluoro-6-methoxy-3-methylimidazo[5,1-c][1,2,4]benzotriazine;
8-fluoro-6-methoxy-3-methyl-1-(3-methylthiophen-2-yl)imidazo[5,1-c][1,2,4]benzotriazine;
8-fluoro-1-(6-fluoro-2-methylpyridin-3-yl)-6-methoxy-3-methylimidazo[5,1-c][1,2,4]benzotriazine;
8-fluoro-1-(6-fluoro-5-methylpyridin-3-yl)-6-methoxy-3-methylimidazo[5,1-c][1,2,4]benzotriazine;
8-fluoro-6-methoxy-1-(5-methoxypyridin-3-yl)-3-methylimidazo[5,1-c][1,2,4]benzotriazine;
8-fluoro-6-methoxy-3-methyl-1-(4-methylpyridin-3-yl)imidazo[5,1-c][1,2,4]benzotriazine;
1-(3,5-dimethyl-1H-pyrazol-4-yl)-8-fluoro-6-methoxy-3-methylimidazo[5,1-c][1,2,4]benzotriazine;
1-[2-chloro-5-(trifluoromethoxy)phenyl]-8-fluoro-6-methoxy-3-methylimidazo[5,1-c][1,2,4]benzotriazine;
1-(2-chloro-5-methoxyphenyl)-8-fluoro-6-methoxy-3-methylimidazo[5,1-c][1,2,4]benzotriazine;
1-(2,4-dimethyl-1,3-thiazol-5-yl)-8-fluoro-6-methoxy-3-methylimidazo[5,1-c][1,2,4]benzotriazine;
4-fluoro-3-(8-fluoro-6-methoxy-3-methylimidazo[5,1-c][1,2,4]benzotriazin-1-yl)benzamide;
8-fluoro-6-methoxy-3-methyl-1-pyridin-4-ylimidazo[5,1-c][1,2,4]benzotriazine;
8-fluoro-6-methoxy-3-methyl-1-(2-methylpyridin-3-yl)imidazo[5,1-c][1,2,4]benzotriazine;
8-fluoro-6-methoxy-3-methyl-1-(3-methylpyridin-4-yl)imidazo[5,1-c][1,2,4]benzotriazine;
6-Chloro-3-methyl-1-(3-methylpyridin-4-yl)-8-(trifluoromethyl)imidazo[5,1-c][1,2,4]benzotriazine;
6-chloro-1-(2,5-dichlorophenyl)-3-methyl-8-(trifluoromethyl)imidazo[5,1-c][1,2,4]benzotriazine;
6-methoxy-3-methyl-1-(3-methylpyridin-4-yl)-8-(trifluoromethyl)benzo[e]imidazo[5,1-c][1,2,4]triazine;
1-(2-Chlorophenyl)-6-methoxy-3-methyl-8-(trifluoromethyl)imidazo[5,1-c][1,2,4]benzotriazine;
6-Methoxy-3-methyl-1-(2-methylpyridin-3-yl)-8-(trifluoromethyl)imidazo[5,1-c][1,2,4]benzotriazine;
6-Methoxy-3-methyl-1-(4-methylpyridin-3-yl)-8-(trifluoromethyl)imidazo[5,1-c][1,2,4]benzotriazine;
6-Methoxy-3-methyl-1-(3-methylthiophen-2-yl)-8-(trifluoromethyl)imidazo[5,1-c][1,2,4]benzotriazine;
6-Methoxy-1-(3-methoxypyridin-4-yl)-3-methyl-8-(trifluoromethyl)benzo[e]imidazo[5,1-c][1,2,4]triazine;
1-(2,5-Dichlorophenyl)-6-methoxy-3-methyl-8-(trifluoromethyl)benzo[e]imidazo[5,1-c][1,2,4]triazine;
1-(3-Fluoro-2-methylphenyl)-6-methoxy-3-methyl-8-(trifluoromethyl)benzo[e]imidazo[5,1-c][1,2,4]triazine;
1-(5-Chloro-2-methoxyphenyl)-6-methoxy-3-methyl-8-(trifluoromethyl)benzo[e]imidazo[5,1-c][1,2,4]triazine;
1-(Furan-3-yl)-6-methoxy-3-methyl-8-(trifluoromethyl)benzo[e]imidazo[5,1-c][1,2,4]triazine;
4-(6-Methoxy-3-methyl-8-(trifluoromethyl)benzo[e]imidazo[5,1-c][1,2,4]triazin-1-yl)-3,5-dimethylisoxazole;
6-Methoxy-3-methyl-1-(thiophen-2-yl)-8-(trifluoromethyl)benzo[e]imidazo[5,1-c][1,2,4]triazine;
1-(5-Fluoro-2-methylphenyl)-6-methoxy-3-methyl-8-(trifluoromethyl)benzo[e]imidazo[5,1-c][1,2,4]triazine;
6-methoxy-3-methyl-1-(4-methylpyridin-3-yl)benzo[e]imidazo[5,1-c][1,2,4]triazin-8-ol;
8-(difluoromethoxy)-6-methoxy-3-methyl-1-(4-methylpyridin-3-yl)benzo[e]imidazo[5,1-c][1,2,4]triazine;
8-(benzyloxy)-6-methoxy-3-methyl-1-(3-methylpyridin-4-yl)imidazo[5,1-c][1,2,4]benzotriazine;
6-methoxy-3-methyl-1-(3-methylpyridin-4-yl)benzo[e]imidazo[5,1-c][1,2,4]triazin-8-ol;
8-(difluoromethoxy)-6-methoxy-3-methyl-1-(3-methylpyridin-4-yl)imidazo[5,1-c][1,2,4]benzotriazine;
8-(benzyloxy)-6-methoxy-3-methyl-1-(2-methylpyridin-3-yl)imidazo[5,1-c][1,2,4]benzotriazine;
6-methoxy-3-methyl-1-(2-methylpyridin-3-yl)imidazo[5,1-c][1,2,4]benzotriazin-8-ol;
8-(difluoromethoxy)-6-methoxy-3-methyl-1-(2-methylpyridin-3-yl)imidazo[5,1-c][1,2,4]benzotriazine;
8-(benzyloxy)-6-methoxy-3-methyl-1-(4-methylpyridin-3-yl)imidazo[5,1-c][1,2,4]benzotriazine;
8-(cyclopropylmethoxy)-6-methoxy-3-methyl-1-(3-methylpyridin-4-yl)benzo[e]imidazo[5,1-c][1,2,4]triazine;
8-(cyclopropylmethoxy)-6-methoxy-3-methyl-1-(2-methylpyridin-3-yl)benzo[e]imidazo[5,1-c][1,2,4]triazine;
8-(difluoromethoxy)-6-methoxy-3-methyl-1-(1,3,5-trimethyl-1H-pyrazol-4-yl)benzo[e]imidazo[5,1-c][1,2,4]triazine;
8-(difluoromethoxy)-1-(3,5-dimethyl-1H-pyrazol-4-yl)-6-methoxy-3-methylbenzo[e]imidazo[5,1-c][1,2,4]triazine;
4-(8-(difluoromethoxy)-6-methoxy-3-methylbenzo[e]imidazo[5,1-c][1,2,4]triazin-1-yl)-3,5-dimethylisoxazole;
5-(8-(difluoromethoxy)-6-methoxy-3-methylbenzo[e]imidazo[5,1-c][1,2,4]triazin-1-yl)-2,4-dimethylthiazole;
8-(difluoromethoxy)-1-(3-fluoro-2-methylphenyl)-6-methoxy-3-methylbenzo[e]imidazo[5,1-c][1,2,4]triazine;
8-(difluoromethoxy)-1-(5-fluoro-2-methylphenyl)-6-methoxy-3-methylbenzo[e]imidazo[5,1-c][1,2,4]triazine;
1-(2-chloro-5-fluorophenyl)-8-(difluoromethoxy)-6-methoxy-3-methylbenzo[e]imidazo[5,1-c][1,2,4]triazine;
1-(5-chloro-2-methoxyphenyl)-8-(difluoromethoxy)-6-methoxy-3-methylbenzo[e]imidazo[5,1-c][1,2,4]triazine;
1-(2,5-dichlorophenyl)-8-(difluoromethoxy)-6-methoxy-3-methylimidazo[5,1-c][1,2,4]benzotriazine;
1-(2-chlorophenyl)-8-(difluoromethoxy)-6-methoxy-3-methylimidazo[5,1-c][1,2,4]benzotriazine;
8-(difluoromethoxy)-6-methoxy-1-(5-methoxypyridin-3-yl)-3-methylimidazo[5,1-c][1,2,4]benzotriazine;
1,8-Bis-(2,5-dichloro-phenyl)-3-methyl-imidazo[5,1-c][1,2,4]benzotriazine;
1,8-Bis-(2-chloro-phenyl)-3-methyl-imidazo[5,1-c][1,2,4]benzotriazine;
1,8-Bis-(2,3-dichloro-phenyl)-3-methyl-imidazo[5,1-c][1,2,4]benzotriazine;
1,8-Bis-(2-fluoro-5-propoxy-phenyl)-3-methyl-imidazo[5,1-c][1,2,4]benzotriazine;
1,8-Bis-(5-butoxy-2-fluoro-phenyl)-3-methyl-imidazo[5,1-c][1,2,4]benzotriazine;
1,8-Bis-(2-fluoro-5-trifluoromethyl-phenyl)-3-methyl-imidazo[5,1-c][1,2,4]benzotriazine;
1,8-Bis-(2-fluoro-5-isopropoxy-phenyl)-3-methyl-imidazo[5,1-c][1,2,4]benzotriazine;
1,8-Bis-(5-ethoxy-2-fluoro-phenyl)-3-methyl-imidazo[5,1-c][1,2,4]benzotriazine;
1-Cyclohexyl-8-(2-cyclohexyl-4-methyl-imidazol-1-yl)-3-methyl-imidazo[5,1-c][1,2,4]benzotriazine;
1-(2,5-Dichloro-phenyl)-3-methyl-8-piperidin-1-yl-imidazo[5,1-c][1,2,4]benzotriazine;
1-(2,5-Dichloro-phenyl)-3-methyl-8-morpholin-4-yl-imidazo[5,1-c][1,2,4]benzotriazine;
1-Isobutyl-8-(2-isobutyl-4-methyl-imidazol-1-yl)-3-methyl-imidazo[5,1-c][1,2,4]benzotriazine;
1-(2-Chloro-phenyl)-3-methyl-8-piperidin-1-yl-imidazo[5,1-c][1,2,4]benzotriazine;
1-(2-Chloro-phenyl)-3-methyl-8-morpholin-4-yl-imidazo[5,1-c][1,2,4]benzotriazine;
1-(2-Chloro-phenyl)-8-imidazol-1-yl-3-methyl-imidazo[5,1-c][1,2,4]benzotriazine;
1-(2-Chloro-phenyl)-3-methyl-8-(4-methyl-piperazin-1-yl)-imidazo[5,1-c][1,2,4]benzotriazine; and
1-(2-Chloro-phenyl)-8-(4-fluoro-benzyloxy)-3-methyl-imidazo[5,1-c][1,2,4]benzotriazine;

or a pharmaceutically acceptable salt thereof.

The chemical compounds described in this specification have been determined using either the ACDLABS 11.0 Name Pro Software (IUPAC Nomenclature of Organic Chemistry Rules; available from Advanced Chemistry Development, Inc.) or the ChemDraw Ultra 9.0.1 software (available from CambridgeSoft). The following contains definitions of terms used in this specification. The initial definition provided for a group or term herein applies to that group or term throughout the present specification, individually or as part of another group, unless otherwise indicated.

At various places in the present specification, substituents of compounds of the invention are disclosed in groups or in ranges. It is specifically intended that the invention include each and every individual subcombination of the members of such groups and ranges. For example, the term “C₁₋₆ alkyl” is specifically intended to individually disclose methyl, ethyl, C₃ alkyl, C₄ alkyl, C₅ alkyl, and C₆ alkyl.

It is further appreciated that certain features of the invention, which are, for clarity, described in the context of separate embodiments, can also be provided in combination in a single embodiment. Conversely, various features of the invention which are, for brevity, described in the context of a single embodiment, can also be provided separately or in any suitable subcombination.

The term “n-membered” where n is an integer typically describes the number of ring-forming atoms in a moiety where the number of ring-forming atoms is n. For example, piperidinyl is an example of a 6-membered heterocycloalkyl ring and 1,2,3,4-tetrahydro-naphthalene is an example of a 10-membered cycloalkyl group.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R. In another example, when an optionally multiple substituent is designated in the form:

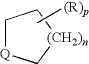

then it is understood that substituent R can occur p number of times on the ring, and R can be a different moiety at each occurrence. It is understood that each R group may replace any hydrogen atom attached to a ring atom, including one or both of the (CH₂)ₙ hydrogen atoms. Further, in the above example, should the variable Q be defined to include hydrogens, such as when Q is said to be CH₂, NH, etc., any floating substituent such as R in the above example, can replace a hydrogen of the Q variable as well as a hydrogen in any other non-variable component of the ring.

For compounds of the invention in which a variable appears more than once, each variable can be a different moiety independently selected from the group defining the variable. For example, where a structure is described having two R groups that are simultaneously present on the same compound, the two R groups can represent different moieties independently selected from the group defined for R.

As used herein, the phrase “optionally substituted” means unsubstituted or substituted. As used herein, the term “substituted” means that a hydrogen atom is removed and replaced by a substitutent. As used herein, the phrase “substituted with oxo” means that two hydrogen atoms are removed from a carbon atom and replaced by an oxygen bound by a double bond to the carbon atom. It is understood that substitution at a given atom is limited by valency.

The terms “alk” or “alkyl” refer to straight or branched chain hydrocarbon groups having 1 to 12 carbon atoms, preferably 1 to 8 carbon atoms or 1 to 6 carbon atoms. The expression “lower alkyl” refers to alkyl groups of 1 to 4 carbon atoms.

The term “alkenyl” refers to straight or branched chain hydrocarbon groups of 2 to 10, preferably 2 to 4, or 2 to 6, carbon atoms having at least one double bond. Where an alkenyl group is bonded to a nitrogen atom, it is preferred that such group not be bonded directly through a carbon bearing a double bond.

The term “alkynyl” refers to straight or branched chain hydrocarbon groups of 2 to 10, preferably 2 to 4, or 2 to 6, carbon atoms having at least one triple bond. Where an alkynyl group is bonded to a nitrogen atom, it is preferred that such group not be bonded directly through a carbon bearing a triple bond.

The term “alkylene” refers to a straight chain bridge of 1 to 5 carbon atoms connected by single bonds (e.g., —(CH₂)ₓ— wherein x is 1 to 5), which may be substituted with 1 to 3 lower alkyl groups.

The term “alkenylene” refers to a straight chain bridge of 2 to 5 carbon atoms having one or two double bonds that is connected by single bonds and may be substituted with 1 to 3 lower alkyl groups. Exemplary alkenylene groups are —CH═CH—CH═CH—; —CH₂—CH═CH—, —CH₂—CH═CH—CH₂—, —C(CH₃)₂CH═CH— and —CH—(C₂H₅)—CH═CH—.

The term “alkynylene” refers to a straight chain bridge of 2 to 5 carbon atoms that has a triple bond therein, is connected by single bonds, and may be substituted with 1 to 3 lower alkyl groups. Exemplary alkynylene groups are —C≡C—, —CH₂—C≡C—, —CH(CH₃)—C≡C— and —C≡C—CH(C₂H₅)CH₂—.

The terms “ar” or “aryl” refer to aromatic mono-, bi- or oligocyclic rings, preferably phenyl, naphthyl and biphenyl. In some embodiments, “ar” or “aryl” has 6 to 12 carbon atoms.

As used herein, the term “alkylamino” refers to a group of formula —NH(alkyl), wherein the alkylene group and alkyl group each have 1 to 6 carbons.

As used herein, the term “alkylcarbamyl” refers to a group of formula —C(O)—NH(alkyl), wherein the alkyl group has 1 to 6 carbons.

As used herein, the term “alkylcarbamyloxy” refers to a group of formula —OC(O)NH(alkyl), wherein the alkyl group has 1 to 6 carbons.

As used herein, the term “alkoxy”, employed alone or in combination with other terms, refers to an group of formula —O-alkyl. Example alkoxy groups include methoxy, ethoxy, propoxy (e.g., n-propoxy and isopropoxy), t-butoxy, and the like.

As used herein, the term “alkoxycarbonyl” refers to a group of formula —C(O)-alkyl.

As used herein, the term “alkylcarbonyl” refers to a group of formula —C(O)-alkyl.

As used herein, the term “alkylsulfinyl” refers to a group of formula —S(O)-alkyl.

As used herein, the term “alkylsulfonyl” refers to a group of formula —S(O)₂-alkyl.

As used herein, the term “alkylthio” refers to a group of formula —S-alkyl.

As used herein, the term “amino”, employed alone or in combination with other terms, refers to a group of formula NH₂.

As used herein, the term “carbamyl” refers to a group of formula —C(O)NH₂.

As used herein, the term “carboxy” refers to a group of formula —C(O)OH.

The terms “cycloalkyl” and “cycloalkenyl” refer to cyclic hydrocarbon groups of 3 to 8 carbon atoms. In some embodiments, one or more carbon atoms of the cycloalkyl or cycloalkenyl ring are oxidized to form a carbonyl group.

As used herein, the term “dialkylamino” refers to a group of formula —N(alkyl)₂, wherein the alkylene group and two alkyl groups each has, independently, 1 to 6 carbons.

As used herein, the term “dialkylcarbamyl” refers to a group of formula —C(O)—N(alkyl)₂, wherein the alkyl groups each has, independently, 1 to 6 carbons.

As used herein, the term “dialkylcarbamyloxy” refers to a group of formula —OC(O)N(alkyl)₂, wherein the alkyl groups each has, independently, 1 to 6 carbon atoms.

As used herein, “haloalkoxy”, employed alone or in combination with other terms, refers to a group of formula —O-haloalkyl. An example haloalkoxy group is OCF₃.

As used herein, the term “haloalkyl”, employed alone or in combination with other terms, refers to an alkyl group having from one halogen atom to 2n+1 halogen atoms which may be the same or different, where “n” is the number of carbon atoms in the alkyl group.

As used herein, the term “heterocycloalkyl” refers to a group of formula-alkyl-heterocyclo.

The terms “halogen” and “halo” refer to fluorine, chlorine, bromine and iodine.

The term “unsaturated ring” includes partially unsaturated and aromatic rings.

The terms “heterocycle”, “heterocyclic” or “heterocyclo” refer to fully saturated or unsaturated, including aromatic (“heteroaryl”) or nonaromatic cyclic groups, for example, 4 to 7 membered monocyclic, 7 to 11 membered bicyclic, or 10 to 15 membered tricyclic ring systems, which have at least one heteroatom in at least one carbon atom-containing ring. Each ring of the heterocyclic group containing a heteroatom may have 1, 2, 3 or 4 heteroatoms selected from nitrogen atoms, oxygen atoms and/or sulfur atoms, where the nitrogen and sulfur heteroatoms may optionally be oxidized and the nitrogen heteroatoms may optionally be quaternized. The heterocyclic group may be attached at any heteroatom or carbon atom of the ring or ring system. In some embodiments, one or more carbon atoms of the heterocyclo ring are oxidized to form a carbonyl group. In some embodiments, the heterocyclo ring has 2 to 12, or 2 to 9 carbon atoms.

Exemplary monocyclic heterocyclic groups include pyrrolidinyl, pyrrolyl, pyrazolyl, oxetanyl, pyrazolinyl, imidazolyl, imidazolinyl, imidazolidinyl, oxazolyl, oxazolidinyl, isoxazolinyl, isoxazolyl, thiazolyl, thiadiazolyl, thiazolidinyl, isothiazolyl, isothiazolidinyl, furyl, tetrahydrofuryl, thienyl, oxadiazolyl, piperidinyl, piperazinyl, 2-oxopiperazinyl, 2-oxopiperidinyl, 2-oxopyrrolodinyl, 2-oxoazepinyl, azepinyl, diazepinyl, 4-piperidonyl, pyridyl, pyrazinyl, pyrimidinyl, pyridazinyl, tetrahydropyranyl, morpholinyl, thiamorpholinyl, thiamorpholinyl sulfoxide, thiamorpholinyl, sulfone, 1,3-dioxolane and tetrahydro-1,1-dioxothienyl, and the like.

Exemplary bicyclic heterocyclic groups include indolyl, benzothiazolyl, benzoxazolyl, benzothienyl, quinuclidinyl, quinolinyl, tetra-hydroisoquinolinyl, isoquinolinyl, benzimidazolyl, benzopyranyl, indolizinyl, benzofuryl, chromonyl, coumarinyl, benzopyranyl, cinnolinyl, quinoxalinyl, indazolyl, pyrrolopyridyl, furopyridinyl (such as furo[2,3-c]pyridinyl, furo[3,2-b]pyridinyl or furo[2,3-b]pyridinyl), dihydroisoindolyl, dihydroquinazolinyl (such as 3,4-dihydro-4-oxo-quinazolinyl), tetrahydroquinolinyl and the like.

Exemplary tricyclic heterocyclic groups include carbazolyl, benzidolyl, phenanthrolinyl, acridinyl, phenanthridinyl, xanthenyl and the like.

As used herein, the term “hydroxyl” refers to a group of formula —OH.

As used herein, the term “nitro” refers to a group of formula —NO₂.

As used herein, the term “sulfinyl”, employed alone or in combination with other terms, refers to —S(O)— group, which is a divalent one-sulfur moiety further bonded to an oxygen atom with a double bond.

As used herein, the term “sulfonyl”, employed alone or in combination with other terms, refers to a —S(O)₂— group, which is a divalent one-sulfur moiety further bonded to two oxygen atoms via double bonds.

As used herein, the term “thio”, employed alone or in combination with other terms, refers to a —S— group, which is a divalent one-sulfur moiety.

Throughout the definitions, the term “Cₙ₋ₘ” is referred to indicate C₁₋₄, C₁₋₆, and the like, wherein n and m are integers and indicate the number of carbons, wherein n-m indicates a range which includes the endpoints.

The compounds of formula I may form salts which are also within the scope of this invention. Reference to a compound of the formula I herein is understood to include reference to salts thereof, unless otherwise indicated. The term “salt(s)”, as employed herein, denotes acidic and/or basic salts formed with inorganic and/or organic acids and bases. Zwitterions (internal or inner salts) are included within the term “salt(s)” as used herein (and may be formed, for example, where the R substituents comprise an acid moiety such as a carboxyl group). Also included herein are quaternary ammonium salts such as alkylammonium salts. Salts of the compounds of the formula I may be formed, for example, by reacting a compound I with an amount of acid or base, such as an equivalent amount, in a medium such as one in which the salt precipitates or in an aqueous medium followed by lyophilization.

Exemplary acid addition salts include acetates (such as those formed with acetic acid or trihaloacetic acid, for example, trifluoroacetic acid), adipates, aliginates, ascorbates, aspartates, benzoates, benzenesulfonates, bisulfates, borates, butyrates, citrates, camphorates, camphorsulfonates, cyclopentanepropionates, digluconates, dodecylsulfates, ethanesulfonates, fumarates, glucoheptanoates, glycerophosphates, hemisulfates, heptanoates, hexanoates, hydrochlorides, hydrobromides, hydroiodides, 2-hydroxyethanesulfonates, lactates, maleates, methanesulfonates, 2-naphthalenesulfonates, nicotinates, nitrates, oxalates, pectinates, persulfates, 3-phenylpropionates, phosphates, picrates, pivalates, propionates, salicylates, succinates, sulfates (such as those formed with sulfuric acid), sulfonates (such as those mentioned herein), tartrates, thiocyanates, toluenesulfonates such as tosylates, undecanoates, and the like.

Exemplary basic salts (formed, for example, where the R substituents comprise an acidic moiety such as a carboxyl group) include ammonium salts, alkali metal salts such as sodium, lithium, and potassium salts, alkaline earth metal salts such as calcium and magnesium salts, salts with organic bases (for example, organic amines) such as benzathines, dicyclohexylamines, hydrabamines, N-methyl-D-glucamines, N-methyl-D-glucamides, t-butyl amines, and salts with amino acids such as arginine, lysine and the like. The basic nitrogen-containing groups may be quaternized with agents such as lower alkyl halides (e.g., methyl, ethyl, propyl, and butyl chlorides, bromides and iodides), dialkyl sulfates (e.g. dimethyl, diethyl, dibutyl, and diamyl sulfates), long chain halides (e.g. decyl, lauryl, myristyl and stearyl chlorides, bromides and iodides), aralkyl halides (e.g. benzyl and phenethyl bromides), and others.

The present invention also includes pharmaceutically acceptable salts of the compounds described herein. As used herein, “pharmaceutically acceptable salts” refers to derivatives of the disclosed compounds wherein the parent compound is modified by converting an existing acid or base moiety to its salt form. Examples of pharmaceutically acceptable salts include, but are not limited to, mineral or organic acid salts of basic residues such as amines; alkali or organic salts of acidic residues such as carboxylic acids; and the like. The pharmaceutically acceptable salts of the present invention include the conventional non-toxic salts of the parent compound formed, for example, from non-toxic inorganic or organic acids. The pharmaceutically acceptable salts of the present invention can be synthesized from the parent compound which contains a basic or acidic moiety by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17^{th} ed., Mack Publishing Company, Easton, Pa., 1985, p. 1418 and Journal of Pharmaceutical Science, 66, 2 (1977), each of which is incorporated herein by reference in its entirety.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

Furthermore, in the case of the compounds of the invention which contain an asymmetric carbon atom, the invention relates to the D form, the L form and D,L mixtures and also, where more than one asymmetric carbon atom is present, to the diastereomeric forms. Those compounds of the invention which contain asymmetric carbon atoms, and which as a rule accrue as racemates, can be separated into the optically active isomers in a known manner, for example using an optically active acid. However, it is also possible to use an optically active starting substance from the outset, with a corresponding optically active or diastereomeric compound then being obtained as the end product.

Compounds of the invention also include tautomeric forms. Tautomeric forms result from the swapping of a single bond with an adjacent double bond together with the concomitant migration of a proton. Tautomeric forms include prototropic tautomers which are isomeric protonation states having the same empirical formula and total charge. Example prototropic tautomers include ketone—enol pairs, amide-imidic acid pairs, lactam—lactim pairs, amide-imidic acid pairs, enamine—imine pairs, and annular forms where a proton can occupy two or more positions of a heterocyclic system, for example, 1H- and 3H-imidazole, 1H-, 2H- and 4H-1,2,4-triazole, 1H- and 2H-isoindole, and 1H- and 2H-pyrazole. Tautomeric forms can be in equilibrium or sterically locked into one form by appropriate substitution.

The compounds described herein can be asymmetric (e.g., having one or more stereocenters). All stereoisomers, such as enantiomers and diastereomers, are intended unless otherwise indicated. Compounds of the present invention that contain asymmetrically substituted carbon atoms can be isolated in optically active or racemic forms. Methods on how to prepare optically active forms from optically active starting materials are known in the art, such as by resolution of racemic mixtures or by stereoselective synthesis. Many geometric isomers of olefins, C═N double bonds, and the like can also be present in the compounds described herein, and all such stable isomers are contemplated in the present invention. Cis and trans geometric isomers of the compounds of the present invention are described and may be isolated as a mixture of isomers or as separated isomeric forms.

Compounds of the invention can also include all isotopes of atoms occurring in the intermediates or final compounds. Isotopes include those atoms having the same atomic number but different mass numbers. For example, isotopes of hydrogen include tritium and deuterium.

Also included are solvates and hydrates of the compounds of formula (I) and solvates and hydrates of their pharmaceutically acceptable salts.

The term “compound” as used herein is meant to include all stereoisomers, geometric iosomers, tautomers, and isotopes of the structures depicted, unless otherwise indicated.

In some embodiments, the compound can be provided as a prodrug. The term “prodrug”, as employed herein, denotes a compound which, upon administration to a subject, undergoes chemical conversion by metabolic or chemical processes to yield a compound of the formula I, or a salt and/or solvate thereof.

In some embodiments, the compounds of the invention, and salts thereof, are substantially isolated. By “substantially isolated” is meant that the compound is at least partially or substantially separated from the environment in which it was formed or detected. Partial separation can include, for example, a composition enriched in the compound of the invention. Substantial separation can include compositions containing at least about 50%, at least about 60%, at least about 70%, at least about 80%, at least about 90%, at least about 95%, at least about 97%, or at least about 99% by weight of the compound of the invention, or salt thereof.

### Pharmaceutical Methods

The compounds according to the invention have been found to have pharmacologically important properties which can be used therapeutically. The compounds of the invention can be used alone, in combination with each other or in combination with other active compounds. Compounds of formula (I) may be inhibitors of phosphodiesterase 2 or 10. It is therefore a part of the subject-matter of this invention that the compounds of the invention and their salts and also pharmaceutical preparations which comprise these compounds or their salts, can be used for treating or preventing disorders associated with, accompanied by and/or covered by phosphodiesterase hyperactivity and/or disorders in which inhibiting phosphodiesterase 2 or 10 is of value. In some embodiments, the compound of formula I is selective for PDE10, meaning that it is a better inhibitor of PDE10 than for any other PDE. In some embodiments, the selective PDE10 inhibitor can reduce PDE10 activity at least 10-fold or at least 100-fold compared to other PDE's. In some embodiments, the compound of formula I is a PDE2 selective inhibitor. In some embodiments, the selective PDE2 inhibitor can reduce PDE2 activity at least 10-fold or at least 100-fold compared to other PDE's.

It is an embodiment of this invention, that compounds of the invention including their salts, solvates and hydrates, can be used for the treatment of central nervous system disorders of mammals including a human.

More particularly, the invention relates to the treatment of neurologic and psychiatric disorders including, but not limited to, (1) mood [affective] disorders; (2) neurotic, stress-related and somatoform disorders including anxiety disorders; (3) disorders comprising the symptom of cognitive deficiency in a mammal, including a human; (4) disorders comprising attention deficits, executive function deficits (working memory deficits), dysfunction of impulse control, extrapyramidal symptoms, disorders that are based on a malfunction of basal ganglia; (5) behavioural and emotional disorders with onset usually occurring in childhood and adolescence; (6) disorders of psychological development; (7) systemic atrophies primarily affecting the central nervous system; (8) extrapyramidal and movement disorders; (9) behavioural syndromes associated with physiological disturbances and physical factors; (10) disorders of adult personality and behaviour; (11) schizophrenia and other psychotic disorders; (12) mental and behavioural disorders due to psychoactive substance use; (13) sexual dysfunction comprising excessive sexual drive; (14) mental retardation; (15) factitious disorders; (16) episodic and paroxysmal disorders, epilepsy; (17) narcolepsy; (18) dementia.

Examples of mood [affective] disorders that can be treated according to the present invention include, but are not limited to, bipolar disorder I depressed, hypomanic, manic and mixed form; bipolar disorder II; depressive disorders, such as single depressive episode or recurrent major depressive disorder, minor depressive disorder, depressive disorder with postpartum onset, depressive disorders with psychotic symptoms; persistent mood [affective] disorders, such as cyclothymia, dysthymia, euthymia; and premenstrual dysphoric disorder.

Examples of disorders belonging to the neurotic, stress-related and somatoform disorders that can be treated according to the present invention include, but are not limited to, anxiety disorders, general anxiety disorder, panic disorder with or without agoraphobia, specific phobia, social phobia, chronic anxiety disorders; obsessive compulsive disorder; reaction to sever stress and adjustment disorders, such as post traumatic stress disorder (PTSD); other neurotic disorders such as depersonalisation-derealisation syndrome.

The phrase “cognitive deficiency” as used here in “disorder comprising as a symptom cognitive deficiency” refers to a subnormal functioning or a suboptimal functioning in one or more cognitive aspects such as memory, intellect, learning and logic ability, or attention and executive function (working memory) in a particular individual comparative to other individuals within the same general age population.

Examples of disorders comprising as a symptom cognitive deficiency that can be treated according to the present invention include, but are not limited to cognitive deficits primarily but not exclusively related to psychosis (schizophrenia), Parkinson's disease, Alzheimer's disease, multi infarct dementia, Lewis body dementia, stroke, frontotemporal dementia, progressive supranuclear palsy, Huntington's disease and in HIV disease, cerebral trauma and drug abuse; mild cognitive disorder and ADHD and Asperger's syndrome and age-associated memory impairment.

Examples of disorders usually first diagnosed in infancy, childhood and adolescence that can be treated according to the present invention include, but are not limited to hyperkinetic disorders, including but not limited to disturbance of activity and attention, attention deficit/hyperactivity disorder (ADHD), hyperkinetic conduct disorder; attention deficit disorder (ADD); conduct disorders, including but not limited to depressive conduct disorder; tic disorders, including but not limited to transient tic disorder, chronic motor or vocal tic disorder, combined vocal and multiple motor tic disorder (de la Tourette), substance induced tic disorders; autistic disorders; excessive masturbation nail-biting, nose-picking and thumb-sucking.

Examples of disorders of psychological development that can be treated according to the present invention include, but are not limited to pervasive developmental disorders, including but not limited to Asperger's syndrome and Rett's syndrome, autistic disorders, childhood autism and overactive disorder associated with mental retardation and stereotyped movements, specific developmental disorder of motor function, specific developmental disorders of scholastic skills.

Examples of systemic atrophies primarily affecting the central nervous system that can be treated according to the present invention include, but are not limited to systemic atrophies primarily affecting the basal ganglia, including but not limited to Huntington's disease, multiple sclerosis, amyotrophic lateral sclerosis.

Examples of movement disorders with malfunction and/or degeneration of basal ganglia that can be treated according to the present invention include, but are not limited to Parkinson's disease; second Parkinsonism, such as postencephalitic Parkinsonism; Parkinsonism comprised in other disorders; Lewis body disease; degenerative diseases of the basal ganglia; other extrapyramidal and movement disorders including but not limited to tremor, essential tremor and drug-induced tremor, myoclonus, chorea and drug-induced chorea, drug-induced tics and tics of organic origion, drug-induced acute dystonia, drug-induced tardive dyskinesia, L-dopa-induced dyskinesia; restless leg syndrome Stiff-man syndrome.

Further examples of movement disorders with malfunction and/or degeneration of basal ganglia that can be treated according to the present invention include, but are not limited to dystonia including but not limited to focal dystonia, multiple-focal or segmental dystonia, torsion dystonia, hemispheric, generalised and tardive dystonia (induced by psychopharmacological drugs). Focal dystonia include cervical dystonia (torticolli), blepharospasm (cramp of the eyelid), appendicular dystonia (cramp in the extremities, like the writer's cramp), oromandibular dystonia and spasmodic dysphonia (cramp of the vocal cord); neuroleptic-induced movement disorders including but not limited to neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, neuroleptic-induced tremor.

Examples of behavioural syndromes associated with physiological disturbances and physical factors according to the present invention include, but are not limited to nonorganic sleep disorders, including but not limited to nonorganic hypersomnia, nonorganic disorder of the sleep-wake schedule; mental and behavioural disorders associated with the puerperium, including but not limited to postnatal and postpartum depression; eating disorders, including but not limited to anorexia nervosa and bulimia nervosa.

Examples of disorders of adult personality and behaviour that can be treated according to the present invention include, but are not limited to personality disorders, including but not limited to emotionally unstable, borderline, obsessive-compulsive, anankastic, dependent and passive-aggressive personality disorder; habit and impulse disorders (impulse-control disorder), including intermittent explosive disorder, pathological gambling, pathological fire-setting (pyromania), pathological stealing (kleptomania), trichotillomania; Münchausen syndrome.

Examples of schizophrenia and other psychotic disorders disorders that can be treated according to the present invention include, but are not limited to, continuous or episodic schizophrenia of different types (for instance paranoid, hebephrenic, catatonic, undifferentiated, residual, and schizophreniform disorders); schizotypal disorders (such as borderline, latent, prepsychotic, prodromal, pseudoneurotic pseudopsychopathic schizophrenia and schizotypal personality disorder); persistent delusional disorders; acute, transient and persistent psychotic disorders; induced delusional disorders; schizoaffective disorders of different type (for instance manic depressive or mixed type); puerperal psychosis and other and unspecified nonorganic psychosis.

Examples of mental and behavioural disorders due to psychoactive substance use that can be treated according to the present invention include, but are not limited to mental and behavioural disorders due to use of alcohol, opioids, cannabinoids, sedatives or hypnotics, cocaine, mental and behavioural disorders due to the use of other stimulants, including caffeine, mental and behavioural disorders due to use of hallucinogens, tobacco, volatile solvents and mental and behavioural disorders due to multiple drug use and use of other psychoactive substances; including but not limited to the following subtype symptoms: harmful use, dependence syndrome, withdrawal state and withdrawal state with delirium.

Examples of dementia that can be treated according to the present invention include, but are not limited to vascular dementia, dementia due to Creutzfeld-Jacob disease, HIV, head trauma, Parkinson's, Huntington's, Pick's disease, dementia of the Alzheimer's type.

The compounds described herein are further useful in the prevention and treatment of obesity, type 2 diabetes (non-insulin dependent diabetes), metabolic syndrome, glucose intolerance, and related health risks, symptoms or disorders. As such, the compounds can also be used to reduce body fat or body weight of an overweight or obese individual.

As used herein, the terms “overweight” and “obese” are meant to refer to adult persons 18 years or older having a greater than ideal body weight (or body fat) measured by the body mass index (BMI). BMI is calculated by weight in kilograms divided by height in meters squared (kg/m²) or, alternatively, by weight in pounds, multiplied by 703, divided by height in inches squared (lbs×703/in²). Overweight individuals typically have a BMI of between 25 and 29, whereas obsess individuals typically have a BMI of 30 or more (see, e.g., National Heart, Lung, and Blood institute, Clinical Guidelines on the Identification, Evaluation, and Treatment of Overweight and Obesity in Adults, The Evidence Report, Washington, D.C.:U.S. Department of Health and Human Services, NIH publication no. 98-4083, 1998). Other means for indicating excess body weight, excess body fat, and obesity include direct measure of body fat and/or waist-to-hip ratio measurements.

The term “metabolic syndrome” is used according to its usual meaning in the art. The American Heart Association characterizes metabolic syndrome as having at least 3 of the 5 below symptoms: 1) Elevated waist circumference (>102 cm (40 inches) in men; >88 cm (35 inches) in women), 2) Elevated triglycerides (>150 mg/dL (>1.7 mmol/L) or drug treatment for elevated triglycerides), 3) Reduced HDL-C (<40 mg/dL (1.03 mmol/L) in men <50 mg/dL (1.3 mmol/L) in women or drug treatment for reduced HDL-C, 4) Elevated blood pressure (>130/85 mmHg or drug treatment for hypertension), and 5) Elevated fasting glucose (>100 mg/dL or drug treatment for elevated glucose). See, Grundy, S. M. et al., Circulation, 2005, 112 (17, e285 (online at circ.ahajournals.org/cgi/reprint/112/17/e285). Metabolic syndrome according to the World Health Organization (See, Alberti et al., Diabet. Med. 15, 539-553, 1998) includes individuals suffering from diabetes, glucose intolerance, low fasting glucose, or insulin resistance plus two or more of 1) High blood pressure (>160/90 mmHg), 2) Hyperlipdemia (triglycerides ≧150 mg/dL or HDL cholesterol <35 mg/dL in men and <39 mg/dL in women), 3) Central obesity (waist-to-hip ratio of >0.90 for men and >0.85 for women or BMI >30 kg/m2), and 4) Microalbuminuria (urinary albumin excretion rate ≧20 μg/min or an albumin-to-creatine ratio ≧20 μg/kg).

The compounds described herein are further useful in the prevention and treatment of disorders associated with enhanced endothelial activity, impaired endothelial barrier and/or enhanced neoangiogenesis, such as septic shock; vascular edema; reduced natriuria pathology; inflammatory diseases, including asthma, rhinitis, arthritis and rheumatoid diseases and autoimmune diseases; acute renal or liver failure, liver dysfunction; neoplasia benign and malignant.

The compounds described herein are further useful in the prevention and treatment of disorders associated with thrombosis or embolism including, but not limited to thrombosis induced tissue infarction in coronary artery disease, in cerebrovascular disease and/or in peripheral vascular disease; stable and unstable angina, transient ischemic attacks, placenta insufficiency thrombosis after surgical procedures, such as bypass, angioplasty, stent placement, heart valve replacement.

The present invention also includes method of treating pain conditions and disorders. Examples of such pain conditions and disorders include, but are not limited to, inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain.

As used herein, the term “treating” or “treatment” refers to one or more of (1) inhibiting the disease; for example, inhibiting a disease, condition or disorder in an individual who is experiencing or displaying the pathology or symptomatology of the disease, condition or disorder (i.e., arresting further development of the pathology and/or symptomatology); and (2) ameliorating the disease; for example, ameliorating a disease, condition or disorder in an individual who is experiencing or displaying the pathology or symptomatology of the disease, condition or disorder (i.e., reversing the pathology and/or symptomatology) such as decreasing the severity of disease.

In some embodiments, administration of a compound of the invention, or pharmaceutically acceptable salt thereof, is effective in preventing the disease; for example, preventing a disease, condition or disorder in an individual who may be predisposed to the disease, condition or disorder but does not yet experience or display the pathology or symptomatology of the disease.

### Pharmaceutical Compositions

The present invention further provides pharmaceutical compositions comprising a compound of formula I or a pharmaceutically acceptable salt thereof. In some embodiments, the composition further comprises a pharmaceutically acceptable carrier.

An effective dose of the compounds according to the invention, or their salts, solvates or prodrugs thereof is used, in addition to physiologically acceptable carriers, diluents and/or adjuvants for producing a pharmaceutical composition. The dose of the active compounds can vary depending on the route of administration, the age and weight of the patient, the nature and severity of the diseases to be treated, and similar factors. The daily dose can be given as a single dose, which is to be administered once, or be subdivided into two or more daily doses, and is as a rule 0.001-2000 mg. Particular preference is given to administering daily doses of 0.1-500 mg, e.g. 0.1-100 mg.

Suitable administration forms are oral, parenteral, intravenous, transdermal, topical, inhalative, intranasal and sublingual preparations. Particular preference is given to using oral, parenteral, e.g. intravenous or intramuscular, intranasal preparations, e.g. dry powder or sublingual, of the compounds according to the invention. The customary galenic preparation forms, such as tablets, sugar-coated tablets, capsules, dispersible powders, granulates, aqueous solutions, alcohol-containing aqueous solutions, aqueous or oily suspensions, syrups, juices or drops, can be used.

Solid medicinal forms can comprise inert components and carrier substances, such as calcium carbonate, calcium phosphate, sodium phosphate, lactose, starch, mannitol, alginates, gelatine, guar gum, magnesium stearate, aluminium stearate, methyl cellulose, talc, highly dispersed silicic acids, silicone oil, higher molecular weight fatty acids, (such as stearic acid), gelatine, agar agar or vegetable or animal fats and oils, or solid high molecular weight polymers (such as polyethylene glycol); preparations which are suitable for oral administration can comprise additional flavourings and/or sweetening agents, if desired.

Liquid medicinal forms can be sterilized and/or, where appropriate, comprise auxiliary substances, such as preservatives, stabilizers, wetting agents, penetrating agents, emulsifiers, spreading agents, solubilizers, salts, sugars or sugar alcohols for regulating the osmotic pressure or for buffering, and/or viscosity regulators.

Examples of such additives are tartrate and citrate buffers, ethanol and sequestering agents (such as ethylenediaminetetraacetic acid and its non-toxic salts). High molecular weight polymers, such as liquid polyethylene oxides, microcrystalline celluloses, carboxymethyl celluloses, polyvinylpyrrolidones, dextrans or gelatine, are suitable for regulating the viscosity. Examples of solid carrier substances are starch, lactose, mannitol, methyl cellulose, talc, highly dispersed silicic acids, high molecular weight fatty acids (such as stearic acid), gelatine, agar agar, calcium phosphate, magnesium stearate, animal and vegetable fats, and solid high molecular weight polymers, such as polyethylene glycol.

Oily suspensions for parenteral or topical applications can be vegetable, synthetic or semisynthetic oils, such as liquid fatty acid esters having in each case from 8 to 22 C atoms in the fatty acid chains, for example palmitic acid, lauric acid, tridecanoic acid, margaric acid, stearic acid, arachidic acid, myristic acid, behenic acid, pentadecanoic acid, linoleic acid, elaidic acid, brasidic acid, erucic acid or oleic acid, which are esterified with monohydric to trihydric alcohols having from 1 to 6 C atoms, such as methanol, ethanol, propanol, butanol, pentanol or their isomers, glycol or glycerol. Examples of such fatty acid esters are commercially available miglyols, isopropyl myristate, isopropyl palmitate, isopropyl stearate, PEG 6-capric acid, caprylic/capric acid esters of saturated fatty alcohols, polyoxyethylene glycerol trioleates, ethyl oleate, waxy fatty acid esters, such as artificial ducktail gland fat, coconut fatty acid isopropyl ester, oleyl oleate, decyl oleate, ethyl lactate, dibutyl phthalate, diisopropyl adipate, polyol fatty acid esters, inter alia. Silicone oils of differing viscosity, or fatty alcohols, such as isotridecyl alcohol, 2-octyldodecanol, cetylstearyl alcohol or oleyl alcohol, or fatty acids, such as oleic acid, are also suitable. It is furthermore possible to use vegetable oils, such as castor oil, almond oil, olive oil, sesame oil, cotton seed oil, groundnut oil or soybean oil.

Suitable solvents, gelatinizing agents and solubilizers are water or water-miscible solvents. Examples of suitable substances are alcohols, such as ethanol or isopropyl alcohol, benzyl alcohol, 2-octyldodecanol, polyethylene glycols, phthalates, adipates, propylene glycol, glycerol, di- or tripropylene glycol, waxes, methyl cellosolve, cellosolve, esters, morpholines, dioxane, dimethyl sulphoxide, dimethylformamide, tetrahydrofuran, cyclohexanone, etc.

Cellulose ethers which can dissolve or swell both in water or in organic solvents, such as hydroxypropylmethyl cellulose, methyl cellulose or ethyl cellulose, or soluble starches, can be used as film-forming agents.

Mixtures of gelatinizing agents and film-forming agents are also perfectly possible. In this case, use is made, in particular, of ionic macromolecules such as sodium carboxymethyl cellulose, polyacrylic acid, polymethacrylic acid and their salts, sodium amylopectin semiglycolate, alginic acid or propylene glycol alginate as the sodium salt, gum arabic, xanthan gum, guar gum or carrageenan. The following can be used as additional formulation aids: glycerol, paraffin of differing viscosity, triethanolamine, collagen, allantoin and novantisolic acid. Use of surfactants, emulsifiers or wetting agents, for example of Na lauryl sulphate, fatty alcohol ether sulphates, di-Na—N-lauryl-β-iminodipropionate, polyethoxylated castor oil or sorbitan monooleate, sorbitan monostearate, polysorbates (e.g. Tween), cetyl alcohol, lecithin, glycerol monostearate, polyoxyethylene stearate, alkylphenol polyglycol ethers, cetyltrimethylammonium chloride or mono-/dialkylpolyglycol ether orthophosphoric acid monoethanolamine salts can also be required for the formulation. Stabilizers, such as montmorillonites or colloidal silicic acids, for stabilizing emulsions or preventing the breakdown of active substances such as antioxidants, for example tocopherols or butylhydroxyanisole, or preservatives, such as p-hydroxybenzoic acid esters, can likewise be used for preparing the desired formulations.

Preparations for parenteral administration can be present in separate dose unit forms, such as ampoules or vials. Use is preferably made of solutions of the active compound, preferably aqueous solution and, in particular, isotonic solutions and also suspensions. These injection forms can be made available as ready-to-use preparations or only be prepared directly before use, by mixing the active compound, for example the lyophilisate, where appropriate containing other solid carrier substances, with the desired solvent or suspending agent.

Intranasal preparations can be present as aqueous or oily solutions or as aqueous or oily suspensions. They can also be present as lyophilisates which are prepared before use using the suitable solvent or suspending agent.

Inhalable preparations can present as powders, solutions or suspensions. Preferably, inhalable preparations are in the form of powders, e.g. as a mixture of the active ingredient with a suitable formulation aid such as lactose.

The preparations are produced, aliquoted and sealed under the customary antimicrobial and aseptic conditions.

As indicated above, the compounds of the invention may be administered as a combination therapy with further active agents, e.g. therapeutically active compounds useful in the treatment of central nervous system disorders. These further compounds may be PDE2 or PDE10 inhibitors or compounds which have an activity which is not based on PDE2 or PDE10 inhibition such as NMDA modulating agents.

For a combination therapy, the active ingredients may be formulated as compositions containing several active ingredients in a single dose form and/or as kits containing individual active ingredients in separate dose forms. The active ingredients used in combination therapy may be co-administered or administered separately.

### Synthesis

Compounds of the invention, including salts thereof, can be prepared using known organic synthesis techniques and can be synthesized according to any of numerous possible synthetic routes.

The reactions for preparing compounds of the invention can be carried out in suitable solvents which can be readily selected by one of skill in the art of organic synthesis. Suitable solvents can be substantially non-reactive with the starting materials (reactants), the intermediates, or products at the temperatures at which the reactions are carried out, e.g., temperatures which can range from the solvent's freezing temperature to the solvent's boiling temperature. A given reaction can be carried out in one solvent or a mixture of more than one solvent. Depending on the particular reaction step, suitable solvents for a particular reaction step can be selected by the skilled artisan.

Preparation of Compounds of the Invention can Involve the Protection and deprotection of various chemical groups. The need for protection and deprotection, and the selection of appropriate protecting groups, can be readily determined by one skilled in the art. The chemistry of protecting groups can be found, for example, in T. W. Greene and P. G. M. Wuts, Protective Groups in Organic Synthesis, 3^{rd} Ed., Wiley & Sons, Inc., New York (1999), which is incorporated herein by reference in its entirety.

Reactions can be monitored according to any suitable method known in the art. For example, product formation can be monitored by spectroscopic means, such as nuclear magnetic resonance spectroscopy (e.g., ¹H or ¹³C), infrared spectroscopy, spectrophotometry (e.g., UV-visible), mass spectrometry, or by chromatographic methods such as high performance liquid chromatography (HPLC) or thin layer chromatography (TLC).

Example synthetic methods for preparing compounds of the invention are provided in the Schemes below. The compounds of the formula I may be prepared by methods such as those illustrated in the following Scheme 1.

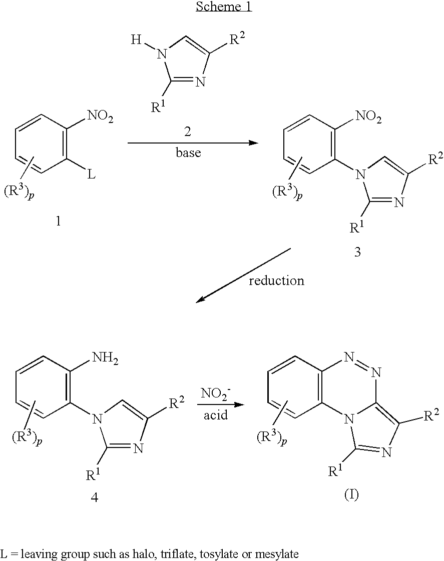

Scheme 1 shows that an appropriately substituted nitro benzene bearing a leaving group L (such as halo) 1 can be reacted with a substituted imidazole 2 in the presence of a base such as carbonates, hydroxides or an non-nucleophilic amine base. The reaction may also be carried out in the presence of a Cu(I) salt. Preferred leaving groups in 1 are F, Cl or Br.

The nitro group of 3 may then be reduced to provide the corresponding amine 4 by methods such as those known in the art, for example, by catalytic hydrogenation, by use of sodium dithionite, SnCl₂, or the like.

The amino group of 4 can then be reacted with a nitrite in the presence of an acid forming the corresponding diazonium salt which immediately forms the final product (I) by intramolecular coupling.

In another approach to the compounds of the formula I (Scheme 2), a 2-haloimidazole as 5 can be utilized in the initial replacement of the leaving group on 1 to provide intermediates 6. This halo group can then be treated with aryl, heteroaryl boronic acids, boronate esters, or organotrifluoroborates (Suzuki coupling) to provide the corresponding aryl or heteroaryl coupled products Ia. In the event that imidazoles of type 7 are used in the displacement of the leaving group in 1, these can be converted to intermediates 9 after which the leaving group L² can be installed, for example through bromination using N-bromosuccinimide. The triazines 9 can then be transformed into the desired compounds of formula Ia. The intermediates 6 can also undergo displacement with nucleophiles such as amines and alcohols (or thiols) in the presence of a base or under Cu(I) catalysis to provide compounds of formula Ib with a heteroatom containing group at R¹.

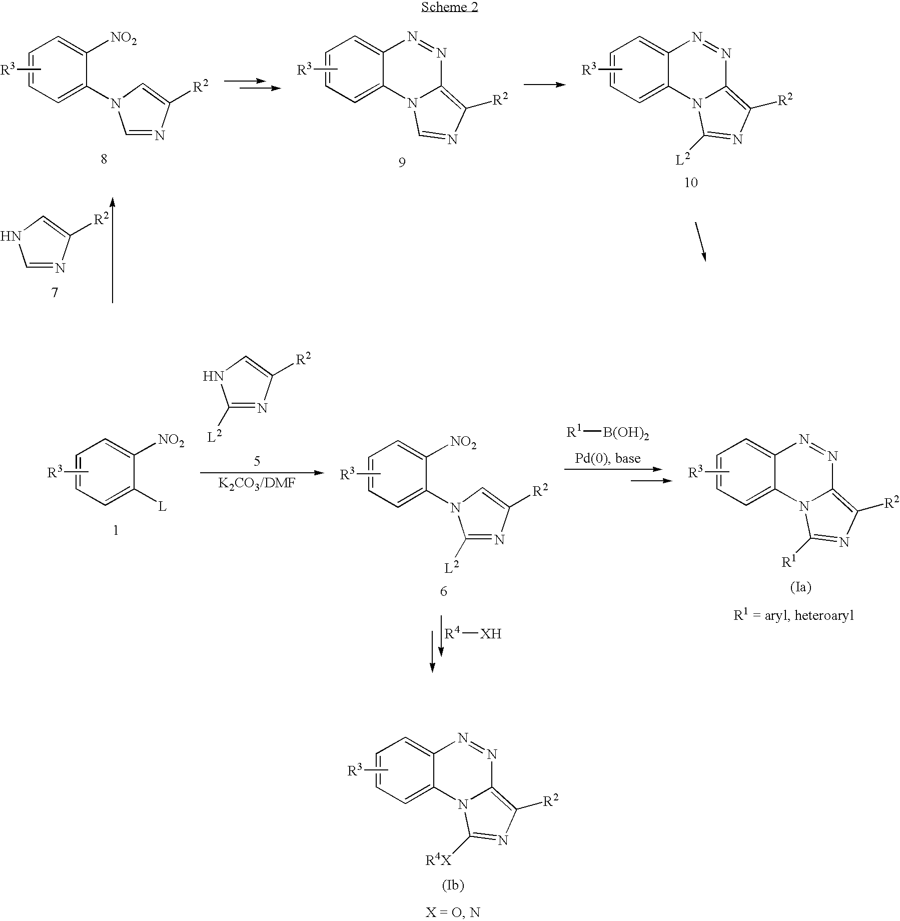

Compounds of formula I can be prepared with various R³ groups through transformations on the benzene ring. For instance, when R³ is a phenolic group, it can be treated with hydrocarbyl halides, tosylates, mesylates and the like to transform the phenolic group to ethers. It should noted that in all of the Schemes described herein, if there are functional groups present on a substituent group such as R¹, R², R³ etc., further modification can be made if appropriate and desired. For example, a CN group can be hydrolyzed to afford an amide group; a carboxylic acid can be converted to a ester, which in turn can be reduced to an alcohol, which in turn can be further modified. In another example, an OH group can be converted into a better leaving group such as mesylate, which in turn is suitable for nucleophilic substitution, such as by CN. Furthermore, an OH group can be subjected to Mitsunobu reaction conditions with phenol, or hetereoaryl alcohol, to afford aryl or heteroaryl ether compounds. Although only few transformations are presented here, similar transformations are within the grasp of a skilled artisan.

In some embodiments, the present invention provides a method of preparing a compound of formula (I), comprising:
(i) reacting an appropriately substituted nitro benzene of formula (I):

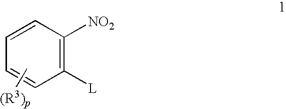

with a substituted imidazole of formula (2):

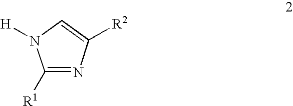

(ii) reducing the nitro group to an amino group;
(iii) reacting the product of step (ii) with a nitrite in the presence of an acid to form the triazine ring structure;
wherein L is a leaving group.

In some embodiments, step (i) is accomplished in the presence of a base.

In some embodiments, the base is selected from a carbonate, hydroxide and amine base.

In some embodiments, L is selected from fluoro, chloro, and bromo.

In some embodiments, the nitro group in (ii) is reduced by catalytic hydrogenation, by use of sodium dithionite, or by use of SnCl₂.

In some embodiments, the acid in (iii) is selected from a mineral acid.

In some embodiments, the acid in (iii) is selected from HCl and H₂SO₄.

The invention will be described in greater detail by way of specific examples. The following examples are offered for illustrative purposes, and are not intended to limit the invention in any manner. Those of skill in the art will readily recognize a variety of non-critical parameters which can be changed or modified to yield essentially the same results.

### EXAMPLES

### Example 1

### 8-fluoro-3-methyl-1-propyl-imidazo[5,1-c][1,2,4]benzotriazine

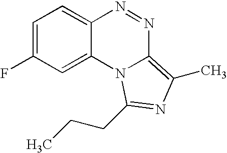

### Step 1: 4-fluoro-2-(4-methyl-2-propyl-imidazol-1-yl)-nitrobenzene

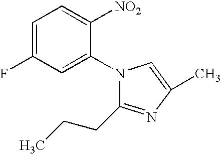

To a suspension of K₂CO₃ (15 g), 4-methyl-2-propyl imidazole (from Ferak Berlin GmbH, Germany; 6.5 g) and 100 ml acetonitrile was added 2,4-difluoro-nitrobenzene (from ABCR GmbH & Co. KG Karlsruhe, Germany or Sigma-Aldrich Co.; 8 g). The reaction mixture was stirred and heated to reflux for 7 h. Then the reaction mixture was filtered off. The solvent was removed and the crude residue was purified by chromatography (dichloromethane (DCM)/butanol 97:3). Yield: 7.2 g; m.p.: 36-39° C.

### Step 2: 1-amino-4-fluoro-2-(4-methyl-2-propyl-imidazol-1-yl)-benzene

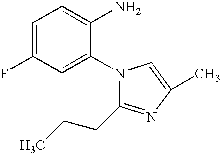

To a solution of 4-fluoro-2-(4-methyl-2-propyl-imidazol-1-yl)-nitrobenzene (2.8 g) and 100 ml ethanol, was added palladium-charcoal (1 g). The reaction mixture was heated to 40° C. and then hydrogenated under pressure (10 to 15 bar). The catalyst was filtrated off at room temperature (RT) and the filtrate was evaporated. To the solid residue methyl tert-butyl ether (MTBE, 20 ml) was added. After stirring for 30 minutes the product was collected by filtration, washed with 5 ml MTBE for 2 times and dried in a dry box with vacuum (40° C.). Yield: 1.0 g; m.p.: 38-42° C.

### Step 3: 8-fluoro-3-methyl-1-propyl-imidazo[5,1-c][1,2,4]benzotriazine

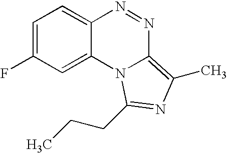

1-amino-4-fluoro-2-(4-methyl-2-propyl-imidazol-1-yl)-benzene (2 g) was dissolved in 25 ml of 1M H₂SO₄. This solution was stirred and cooled with ice. A solution of sodium nitrite (1 g) in 10 ml water was added to the mixture over a period of time of 30 minutes. It was stirred for additional 2 h at about 0° C. A crude yellow product precipitated. It was filtered off and re-crystallized from iso-propanol. Yield: 3.6 g; m.p.: 124.5-126.5° C.; MS [M+H]⁺: 245.

### Example 2

### 7-methoxy-3-methyl-1-propyl-imidazo[5,1-c][1,2,4]benzotriazine

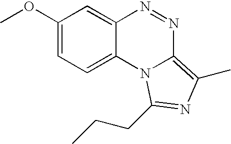

This compound was prepared as described in Example 1 by replacing 2,4-difluoro-nitrobenzene with 1-fluoro-4-methoxy-2-nitrobenzene in step 1. m.p.: 159-162° C.; MS [M+H]⁺: 257.

### Example 3

### 1-ethyl-8-fluoro-3-methyl-imidazo[5,1-c][1,2,4]benzotriazine

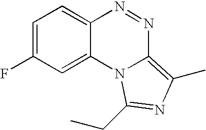

This compound was prepared as described in Example 1 by replacing 4-methyl-2-propyl imidazole with 4-methyl-2-ethyl imidazole (from ABCR GmbH & Co. KG Karlsruhe, Germany) in step 1. m.p.: 187-190° C.; MS [M+H]⁺: 231.

### Example 4

### 1-cyclohexyl-8-fluoro-3-methyl-imidazo[5,1-c][1,2,4]benzotriazine

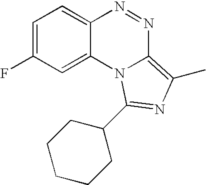

### Step 1. 4-methyl-2-cyclohexyl imidazole

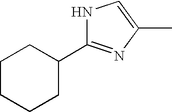

6.5 g cyclohexyl aldehyde was stirred with 44 ml ethanol and 23 ml conc. NH₃.H₂O (32%) at RT for 30 minutes. The mixture was heated to 50 to 60° C. 11.5 ml methyl glyoxal were added drop-wise. The clear solution was stirred at 55° C. for 6 h. At RT, 40 ml water were added. The solvent ethanol was distilled off under reduced pressure. The crude product precipitated. It was filtered off washed with 2×30 ml water and dried at 30° C. Yield 7.7 g.

### Step 2. 1-cyclohexyl-8-fluoro-3-methyl-imidazo[5,1-c][1,2,4]benzotriazine

This compound was prepared as described in Example 1 by replacing 4-methyl-2-propyl imidazole with 4-methyl-2-cyclohexyl imidazole in step 1. m.p.: 246-250° C. (decomp.); MS [M+H]⁺: 285.

### Example 5

### 1-(2,5-dichlorophenyl)-8-methoxy-3-methyl-imidazo[5,1-c][1,2,4]benzotriazine

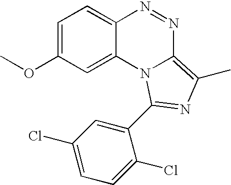

### Step 1. 2-(2,5-dichlorophenyl)-4-methyl-1H-imidazole

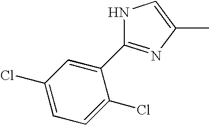

10.0 g 2,5-dichlorobenzaldehyde was stirred with 44 ml ethanol and 23 ml conc. NH₃.H₂O (32%) at RT for 30 minutes. The mixture was heated to 50 to 60° C. 11.5 ml methyl glyoxal were added drop-wise. The clear solution was stirred at 55° C. for 6 h. At RT, 40 ml water were added. The solvent ethanol was distilled off under reduced pressure. The crude product precipitated. It was filtered off washed with 2×30 ml water and dried at 30° C. Yield 12.0 g.

### Step 2. 1-(2,5-dichlorophenyl)-8-fluoro-3-methyl-imidazo[5,1-c][1,2,4]benzotriazine

This compound was prepared as described in Example 1 by replacing 2,4-difluoro-nitrobenzene with 2-fluoro-4-methoxy-1-nitrobenzene and replacing 4-methyl-2-propyl imidazole with 4-(2,5-dichlorophenyl)-2-methyl-1H-imidazole in step 1. m.p.: 165-168° C.; MS [M+H]⁺: 359.

### Example 6

### 1-(2,5-dichlorophenyl)-7-fluoro-3-methyl-imidazo[5,1-c][1,2,4]benzotriazine

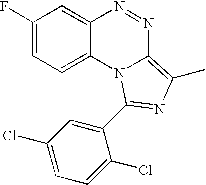

This compound was prepared as described in Example 1 by replacing 2,4-difluoro-nitrobenzene with 1,4-difluoro-2-nitrobenzene (from ABCR GmbH & Co. KG Karlsruhe, Germany) and replacing 4-methyl-2-propyl imidazole with 4-(2,5-dichlorophenyl)-2-methyl-1H-imidazole in step 1. m.p.: 186-190° C.; MS [M+H]⁺: 347.

### Example 7

### 1-(2,5-dichlorophenyl)-7-methoxy-3-methyl-imidazo[5,1-c][1,2,4]benzotriazine

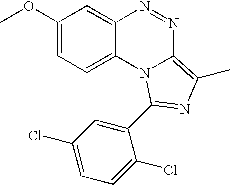

This compound was prepared as described in Example 1 by replacing 2,4-difluoro-nitrobenzene with 1-fluoro-4-methoxy-2-nitrobenzene and replacing 4-methyl-2-propyl imidazole with 4-(2,5-dichlorophenyl)-2-methyl-1H-imidazole in step 1. m.p.: 144-147° C.; MS [M+H]⁺: 359.

The examples in Table 1 were prepared according to procedure described for Example 1.

TABLE 1

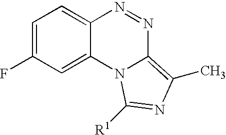

| | | | MS | |
|---|---|---|---|---|
| Example # | R¹ | Name | [M + H]⁺ | m. p (° C.) |
| 8 | 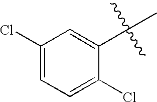 | 1-(2,5-dichlorophenyl)-8- fluoro-3-methylbenzo[e] imidazo[5,1-c][1,2,4]triazine | 347 | 146-149 |
| 9 | iso-butyl | 8-fluoro-1-isobutyl-3- | 359 | 133-135 |
| | | methylbenzo[e]imidazo[5, | | |
| | | 1-c][1,2,4]triazine | | |
| 10 | sec-butyl | 1-sec-butyl-8-fluoro-3- | 259 | 169-171.5 |
| | | methylbenzo[e]imidazo[5,1- | | |
| | | c][1,2,4]triazine | | |
| 11 | H | 8-fluoro-3-methylbenzo | 203 | 230 |
| | | [e]imidazo[5,1-c] | | |
| | | [1,2,4]triazine | | |
| 12 | 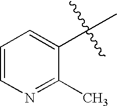 | 8-fluoro-3-methyl-1-(2- methylpyridin-3-yl) benzo[e]imidazo[5,1-c] [1,2,4]triazine | 294 | 207-210 |
| 13 | 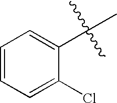 | 1-(2-chlorophenyl)-8-fluoro- 3-methylbenzo[e]imidazo[5, 1-c][1,2,4]triazine | 313 | 189-192 |
| 14 | 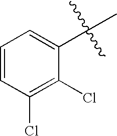 | 1-(2,3-dichlorophenyl)-8- fluoro-3-methylbenzo[e] imidazo[5,1-c][1,2,4]triazine | 347 | 193-196 |
| 15 | 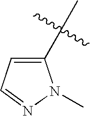 | 8-fluoro-3-methyl-1-(1- methyl-1H-pyrazol-5- yl)benzo[e]imidazo[5,1- c][1,2,4]triazine | 283 | 217-220 |
| 16 | 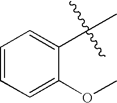 | 8-fluoro-1-(2- methoxyphenyl)-3- methylbenzo[e]imidazo [5,1-c][1,2,4]triazine | 309 | 196-198 |
| 17 | 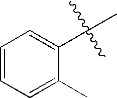 | 8-fluoro-3 -methyl-1-o- tolylbenzo[e]imidazo [5,1-c][1,2,4]triazine | 293 | 192-195 |
| 18 | 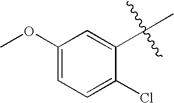 | 1-(2-chloro-5- methoxyphenyl)-8-fluoro-3- methylbenzo[e]imidazo [5,1-c][1,2,4]triazine | 343 | 190-192 |
| 19 | 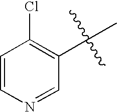 | 1-(4-chloropyridin-3-yl)-8- fluoro-3-methylbenzo [e] imidazo[5,1-c][1,2,4]triazine | 314 | 141-144 |
| 20 | 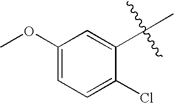 | 8-fluoro-1-(2-fluoro-5- isopropoxyphenyl)-3- methylbenzo[e]imidazo [5,1-c][1,2,4]triazine | 355 | 148-150.5 |
| 21 | 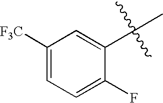 | 8-fluoro-1-(2-fluoro-5- (trifluoromethyl)phenyl)-3- methylbenzo[e]imidazo [5,1-c][1,2,4]triazine | 365 | 176-179 |
| 22 | 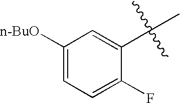 | 1-(5-butoxy-2-fluorophenyl)- 8-fluoro-3-methylbenzo [e]imidazo[5,1-c] [1,2,4]triazine | 369 | 148-151 |
| 23 | 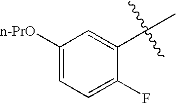 | 8-fluoro-1-(2-fluoro-5- propoxyphenyl)-3- methylbenzo[e]imidazo [5,1-c][1,2,4]triazine | 355 | 153-155 |
| 24 | 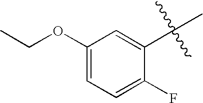 | 1-(5-ethoxy-2-fluorophenyl)- 8-fluoro-3-methylbenzo [e]imidazo[5,1-c] [1,2,4]triazine | 341 | 189-191 |

The examples in Table 2 were prepared according to procedure described for Example 1.

TABLE 2

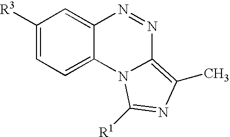

| | | | | MS | |
|---|---|---|---|---|---|
| Example # | R¹ | R³ | Name | [M + H]⁺ | m. p (° C.) |
| 25 | 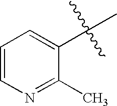 | F | 7-fluoro-3-methyl-1-(2- methylpyridin-3-yl) benzo[e]imidazo[5,1-c] [1,2,4]triazine | 294 | 169-172 |
| 26 | 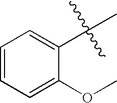 | F | 7-fluoro-1-(2- methoxyphenyl)-3- methylbenzo[e]imidazo [5,1-c][1,2,4]triazine | 309 | 249-251 |
| 27 | 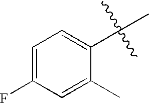 | F | 7-fluoro-1-(4-fluoro-2- methylphenyl)-3- methylbenzo[e]imidazo [5,1-c][1,2,4]triazine | 311 | 181-184 |
| 28 | 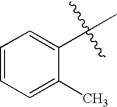 | F | 7-fluoro-3-methyl-1-o- tolylbenzo[e]imidazo [5,1-c][1,2,4]triazine | 293 | 149-152 |
| 29 | 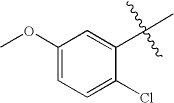 | F | 1-(2-chloro-5- methoxyphenyl)-7-fluoro-3- methylbenzo[e]imidazo [5,1-c][1,2,4]triazine | 343 | 158-161.5 |
| 30 | 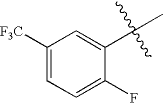 | F | 7-fluoro-1-(2-fluoro-5- (trifluoromethyl)phenyl)-3- methylbenzo[e]imidazo [5,1-c][1,2,4]triazine | 365 | 205-206 |
| 31 | 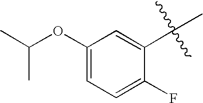 | F | 7-fluoro-1-(2-fluoro-5- isopropoxyphenyl)-3- methylbenzo[e]imidazo [5,1-c][1,2,4]triazine | 355 | 133-136 |
| 32 | 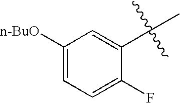 | F | 1-(5-butoxy-2- fluorophenyl)-7-fluoro-3- methylbenzo[e]imidazo [5,1-c][1,2,4]triazine | 369 | 91-94 |
| 33 | 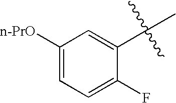 | F | 7-fluoro-1-(2-fluoro-5- propoxyphenyl)-3- methylbenzo[e]imidazo [5,1-c][1,2,4]triazine | 355 | 119-122 |
| 34 | 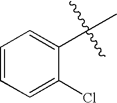 | OMe | 1-(2-chlorophenyl)-7- methoxy-3-methylbenzo[e] imidazo[5,1-c][1,2,4]triazine | 325 | 163-165 |
| 35 | H | OMe | 7-methoxy-3-methylbenzo | 215 | 212-215 |
| | | | [e]imidazo[5,1-c] | | |
| | | | [1 ,2,4]triazine | | |
| 36 | 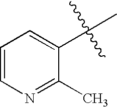 | OMe | 7-methoxy-3 -methyl-1 (2-methylpyridin-3-yl) benzo[e]imidazo [5,1-c][1,2,4]triazine | 306 | 202-205 |

The examples in Table 3 were prepared according to procedure described for Example 1.

TABLE 3

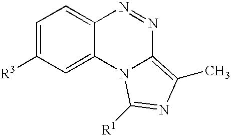

| Example | | | | MS | m.p |
|---|---|---|---|---|---|
| # | R¹ | R³ | Name | [M + H]⁺ | (° C.) |
| 37 | 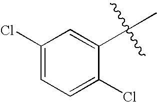 | Cl | 8-chloro-1-(2,5-dichlorophenyl)-3- methylbenzo[e]imidazo[5,1- c][1,2,4]triazine | 363 | 188-190 |
| 38 | 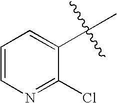 | Cl | 8-chloro-3-methyl-1-(2-methylpyridin- 3-yl)benzo[e]imidazo[5,1- c][1,2,4]triazine | 310 | 235-236 |
| 39 | 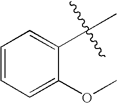 | Cl | 8-chloro-1-(2-methoxyphenyl)-3- methylbenzo[e]imidazo[5,1- c][1,2,4]triazine | 325 | 179-182 |
| 40 | 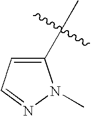 | Cl | 8-chloro-3-methyl-1-(1-methyl-1H- pyrazol-5-yl)benzo[e]imidazo[5,1- c][1,2,4]triazine | 299 | 230-231 |
| 41 | 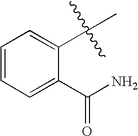 | Cl | 2-(8-chloro-3- methylbenzo[e]imidazo[5,1- c][1,2,4]triazin-1-yl)benzamide | 338 | 249-251 |
| 42 | H | Cl | 8-chloro-3- methylbenzo[e]imidazo[5,1- c][1,2,4]triazine | 219 | 210 |
| 43 | 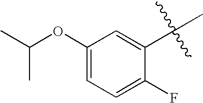 | Cl | 8-chloro-1-(2-fluoro-5- isopropoxyphenyl)-3- methylbenzo[e]imidazo[5,1- c][1,2,4]triazine | 371 | 174-177 |
| 44 | 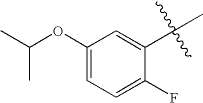 | OMe | 1-(2-fluoro-5-isopropoxyphenyl)-8- methoxy-3- methylbenzo[e]imidazo[5,1- c][1,2,4]triazine | 367 | 204-207 |

### Example 45

### 6,8-dimethoxy-3-methyl-1-(4-methylpyridin-3-yl)imidazo[5,1-c][1,2,4]benzotriazine

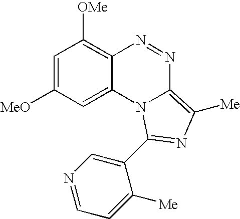

### Step 1: 2-bromo-1-(3,5-difluoro-2-nitrophenyl)-4-methyl-1H-imidazole

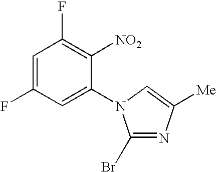

A mixture of 1,3,5-trifluoro-2-nitrobenzene (2.2 mL, 18.6 mmol), 2-bromo-4-methylimidazole (prepared according to EP 0514198; 3 g, 18.6 mmol) and K₂CO₃ (5.66 g, 41 mmol) in 80 mL DMF was stirred at RT overnight. The mixture was diluted with ethyl acetate and washed with water. Standard work up procedure followed by column purification using 10% ethyl acetate in dichloromethane as eluent provided 3.18 g (54% yield) of the product as a yellow powder. EIMS 317.9 [M+H]⁺.

### Step 2: 2-bromo-1-(3,5-dimethoxy-2-nitrophenyl)-4-methyl-1H-imidazole

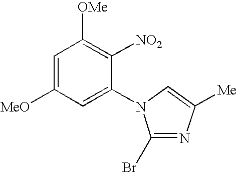

To a solution of 2-bromo-1-(3,5-difluoro-2-nitrophenyl)-4-methyl-1H-imidazole (2.98 g, 9.4 mmol) in 40 mL MeOH was added freshly powdered KOH (2.5 g, 44.6 mmol) at RT under nitrogen. The resulting mixture was stirred at 55° C. for 2 h, cooled to RT, diluted with dichloromethane and poured into water. Standard work up and condensation on rotavap provided 3.35 g (100% yield) of the product as an off-white solid. EIMS 342.0 [M+H]⁺.

### Step 3: 3-(1-(3,5-dimethoxy-2-nitrophenyl)-4-methyl-1H-imidazol-2-yl)-4-methylpyridine

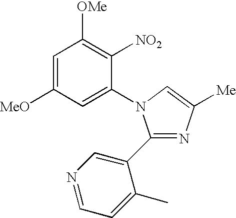

To a mixture of 2-bromo-1-(3,5-dimethoxy-2-nitrophenyl)-4-methyl-1H-imidazole (1.1 g, 3.2 mmol), 4-methyl-3-pyridylboronic acid (commercially available from Combi-Blocks Inc.; 876 mg, 6.4 mmol), K₂CO₃ (1.32 g, 9.6 mmol) and Pd(PPh₃)₄ (186 mg, 0.161 mmol) in a 20 mL vial was added 60 mL of 1,4-dioxane and 20 mL of water under nitrogen. The mixture was stirred at 90° C. for 4 h and cooled to RT. Standard work up followed by column purification provided 788 mg (69% yield) of the desired product as an off-white powder. EIMS 355.1 [M+H]⁺.

### Step 4: 2,4-dimethoxy-6-(4-methyl-2-(4-methylpyridin-3-yl)-1H-imidazol-1-yl)aniline

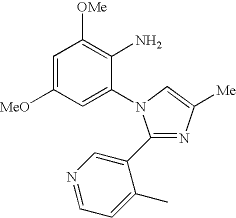

To a mixture of 3-(1-(3,5-dimethoxy-2-nitrophenyl)-4-methyl-1H-imidazol-2-yl)-4-methylpyridine (786 mg, 2.22 mmol) and 10% Pd/C (126 mg, 0.111 mmol) in a 100 mL RB flask (connected with a condenser) was added 10 mL THF, followed by slow addition of 10 mL MeOH with stirring. HCOONH₄ (832 mg, 12.21 mmol) was added in three portions into the stirring mixture and the final mixture was stirred at RT for 10 min (gas released) and then warmed to 50° C. for 1 h. The reaction was cooled to RT and filtered through Celite. Solvent was evaporated by rotovap and the residue was partitioned between water (˜50 mL) and ethyl acetate (˜80 mL). Aqueous phase was extracted with ethyl acetate (3×50 mL). The combined organic phase was dried over MgSO₄. Condensation followed by column purification using 10% methanol in dichloromethane as eluent provided 460 mg (64% yield) of the product as an off-white powder. EIMS 325.1 [M+H]⁺.

### Step 5: 6,8-dimethoxy-3-methyl-1-(4-methylpyridin-3-yl)imidazo[5,1-c][1,2,4] benzotriazine

To a solution of 2,4-dimethoxy-6-(4-methyl-2-(4-methylpyridin-3-yl)-1H-imidazol-1-yl)aniline (102 mg, 0.314 mmol) in 4 mL of AcOH was added a solution of NaNO₂ (33 mg, 0.48 mol) in 0.6 mL of water at RT. The resulting mixture was stirred at RT for 1 h and quenched with NaHCO₃ solution (make sure pH>7). Extraction with ethyl acetate and condensation on rotavap provided the clean product (85 mg, 81% yield) as a yellow powder. EIMS 336.1 [M+H]⁺.

The examples in Table 4 were prepared according to procedure described for Example 126.

TABLE 4

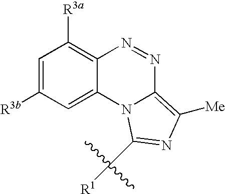

| Example | | | | | MS | m.p |
|---|---|---|---|---|---|---|
| # | R¹ | R^{3a} | R^{3b} | Name | [M + H]⁺ | (° C.) |
| 46 | 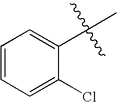 | OMe | OMe | 1-(2-chlorophenyl)-7,8- dimethoxy-3- methylbenzo[e]imidazo[5,1- c][1,2,4]triazine | 355 | 269-272 |
| 47 | 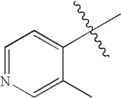 | OMe | OMe | 6,8-dimethoxy-3-methyl-1-(3- methylpyridin-4-yl)-imidazo[5,1- c][1,2,4]benzotriazine | 336.1 | |
| 48 | 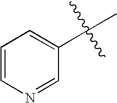 | OMe | OMe | 6,8-dimethoxy-3-methyl-1-(2- methylpyridin-3-yl)imidazo[5,1- c][1,2,4]benzotriazine | 336.1 | |
| 49 | 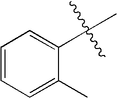 | OMe | OMe | 7,8-dimethoxy-3-methyl-1-o- tolylbenzo[e]imidazo[5,1- c][1,2,4]triazine | 335 | 219-221 |
| 50 | 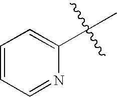 | OMe | OMe | 7,8-dimethoxy-3-methyl-1- (pyridin-2- yl)benzo[e]imidazo[5,1- c][1,2,4]triazine | 322 | 199-202 |
| 51* | 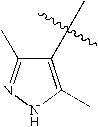 | OMe | OMe | 1-(3,5-dimethyl-1H-pyrazol-4-yl)- 6,8-dimethoxy-3- methylimidazo[5,1- c][1,2,4]benzotriazine | 339.2 | |
| 52 | 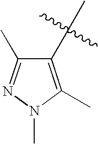 | OMe | OMe | 6,8-dimethoxy-3-methyl-1-(1,3,5- trimethyl-1H-pyrazol-4- yl)imidazo[5,1- c][1,2,4]benzotriazine | 353.2 | |
| 53 | iso-butyl | OMe | OMe | 1-isobutyl-7,8-dimethoxy-3- methylbenzo[e]imidazo[5,1- c][1,2,4]triazine | 301 | 196-199 |
| 54 | Br | OMe | OMe | 1-bromo-7,8-dimethoxy-3- methylbenzo[e]imidazo[5,1- c][1,2,4]triazine | 323 | 200 |
| 55 | 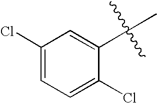 | OMe | OMe | 1-(2,5-dichlorophenyl)-7,8- dimethoxy-3- methylbenzo[e]imidazo[5,1- c][1,2,4]triazine | 389 | 175 |
| 56 | 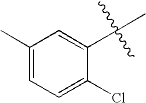 | OMe | OMe | 1-(2-chloro-5-methylphenyl)-7,8- dimethoxy-3- methylbenzo[e]imidazo[5,1- c][1,2,4]triazine | 369 | 247-249 |
| 57 | 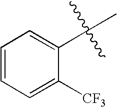 | OMe | OMe | 7,8-dimethoxy-3-methyl-1-(2- (trifluoromethyl)phenyl)benzo[e] imidazo[5,1-c][1,2,4]triazine | 389 | 147-150 |
| 58 | 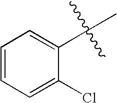 | F | F | 1-(2-chlorophenyl)-7,8-difluoro-3- methylbenzo[e]imidazo[5,1- c][1,2,4]triazine | 331 | 157-159 |
| 59 | iso-butyl | F | F | 7,8-difluoro-1-isobutyl-3- methylbenzo[e]imidazo[5,1- c][1,2,4]triazine | 277 | 123-125.5 |
| 60 | 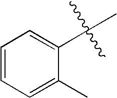 | OMe | H | 6-methoxy-3-methyl-1-(2- methylphenyl)imidazo[5,1- c][1,2,4]benzotriazine | 305.1 | |
| 61 | 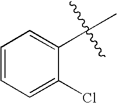 | OMe | H | 1-(2-chlorophenyl)-6-methoxy-3- methylbenzo[e]imidazo[5,1- c][1,2,4]triazine | 325 | 188-191 |
| 62 | 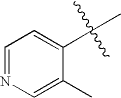 | OMe | H | 6-methoxy-3-methyl-1-(3- methylpyridin-4-yl)imidazo[5,1- c][1,2,4]benzotriazine | 306.1 | |
| 63** | 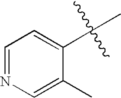 | OMe | A | 6-methoxy-3-methyl-1-(3- methylpyridin-4-yl)-8-morpholin- 4-ylimidazo[5,1- c][1,2,4]benzotriazine | 391.1 | |
| 64 | 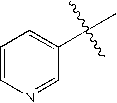 | OMe | A | 6-methoxy-3-methyl-1-(2- methylpyridin-3-yl)-8-morpholin- 4-ylimidazo[5,1- c][1,2,4]benzotriazine | 391.1 | |
| 65 | 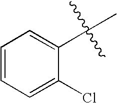 | OMe | A | 1-(2-chlorophenyl)-6-methoxy-3- methyl-8-morpholin-4- ylimidazo[5,1- c][1,2,4]benzotriazine | 410.1 | |
| 66 | 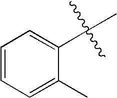 | OMe | A | 6-methoxy-3-methyl-1-(2- methylphenyl)-8-morpholin-4- ylimidazo[1,5- c][1,2,4]benzotriazine | 390.2 | |

*Reduction performed using procedure from step 4 of Examples 67-98.
**A = morpholinyl.

### Examples 67-98

The examples 67-98 were prepared according to procedure described below and summarized in Table 5.

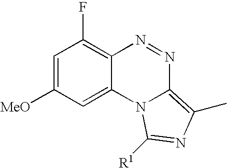

### Step 1: 3,5-difluoro-4-nitrophenol [A1] and 3,5-difluoro-2-nitrophenol [A2]

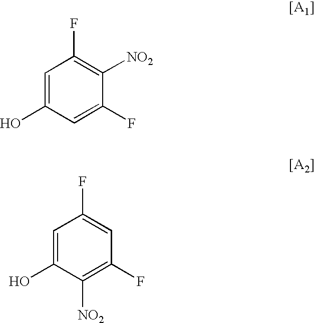

To a methylene chloride (150 mL) solution of 3,5-difluorophenol (14.08 g, 108 mmol) was added fuming nitric acid (>90%, 15 mL) in drop-wise (The addition speed significantly affects the ratio of A1/A2—the slower the addition, the more product of A1) under N₂ at 0° C. After addition, the resulting solution was stirred at the same temperature for 2 h. The reaction was poured into cold water. The organic layer was separated and the aqueous layer was extracted with methylene chloride. Combined organic layer was washed with brine and dried over magnesium sulfate. Condensation under vacuo and column chromatography using 10-30% ethyl acetate in hexane provided 3,5-difluoro-4-nitrophenol [A1] as a thick yellow oil (5.1 g, 27% yield) and 3,5-difluoro-2-nitrophenol [A2] as a yellow solid (8.0 g, 42% yield).

### Step 2: 1,3-difluoro-5-methoxy-2-nitrobenzene

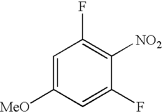

To a mixture of 3,5-difluoro-4-nitrophenol (5.1 g, 29 mmol) and potassium carbonate (8.0 g, 58 mmol) in N,N-dimethylformamide (45 mL) was added MeI (2.2 mL, 34.8 mmol) at RT. The resulting mixture was stirred at RT overnight. Most solvent was removed under vacuo. The residue was diluted with water and ethyl acetate. Organic layer was separated and the aqueous layer was extracted with ethyl acetate. Combined organic layer was washed with brine and dried over magnesium sulfate. Condensation under vacuo and column chromatography using 20% ethyl acetate in hexane provided the product 1,3-difluoro-5-methoxy-2-nitrobenzene (4.4 g, 81% yield)

### Step 3: 2-bromo-1-(3-fluoro-5-methoxy-2-nitrophenyl)-4-methyl-1H-imidazole

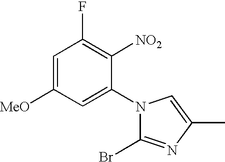

To a mixture of 1,3-difluoro-5-methoxy-2-nitrobenzene (9 g, 47.6 mmol) and potassium carbonate (14.5 g, 104.72 mmol) in 240 mL DMF was added 2-bromo-4-methylimidazole (7.7 g, 47.6 mmol) at RT. The resulting mixture was stirred at RT overnight. Majority of solvent was removed by rotavap. The residue was diluted with ethyl acetate and washed with water. Extraction with ethyl acetate, condensation on rotavap, followed by column chromatography using 5-10% ethyl acetate in dichloromethane as eluent to provide 4.8 g (31%) titled product as a yellow solid.

### Step 4: 2-(2-bromo-4-methyl-1H-imidazol-1-yl)-4-methoxy-6-fluoroaniline

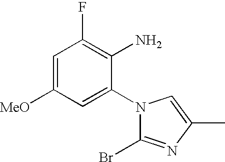

A mixture of 2-bromo-1-(3-fluoro-5-methoxy-2-nitrophenyl)-4-methyl-1H-imidazole (1 eq) and iron powder (6 eq) in a mixture solvent of AcOH and EtOH (1:1) was stirred at 100° C. under nitrogen for 3 h. The hot mixture was filtered through Celite and washed (3×) with ethyl acetate. Condensed on rotavap and the residue was partitioned between sodium carbonate solution and ethyl acetate. Extracted with ethyl acetate and purified by column to provide the desired product.

### Step 5: 1-bromo-6-fluoro-8-methoxy-3-methylbenzo[e]imidazo[5,1-c][1,2,4]triazine

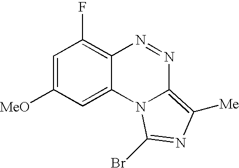

To a solution of 2-(2-bromo-4-methyl-1H-imidazol-1-yl)-4-methoxy-6-fluoroaniline (1 eq) in AcOH was added NaNO₂ (1.5 eq) in water (1 mL/1 mmol NaNO₂) solution at RT. The resulting solution was stirred at RT for 1 h. Worked up with sodium bicarbonate solution to make sure pH>7 and extracted with ethyl acetate. Purification by column provided 1-bromo-6-fluoro-8-methoxy-3-methylbenzo[e]imidazo[5,1-c][1,2,4]triazine.

### Step 6: General Suzuki Coupling: Preparation of Compounds in Table 5

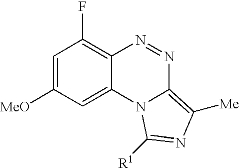

To a mixture of 1-bromo-6-fluoro-8-methoxy-3-methylbenzo[e]imidazo[5,1-c][1,2,4]triazine (1 mmol), boronic acid (commercially available from Sigma-Aldrich Corporation, Boron Molecular Inc., SynQuest Laboratories, and Combi-Blocks Inc.; 2 mmol), K₂CO₃ (3 mmol) and Pd(PPh₃)₄ (0.05 mmol) was added 20 mL of 1,4-dioxane and 7 mL of water under nitrogen. The mixture was stirred at 90° C. for 4 h and cooled to RT. Standard work up followed by column purification provided (70-80% yield) of the desired product.

TABLE 5

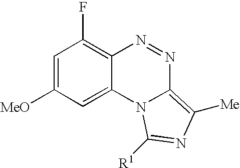

| Example | | | MS |
|---|---|---|---|
| # | R¹ | Name | [M + H]⁺ |
| 67 | 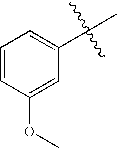 | 6-fluoro-8-methoxy-1-(3-methoxyphenyl)-3- methylimidazo[5,1-c][1,2,4]benzotriazine | 339.22 |
| 68 | 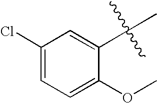 | 1-(5-chloro-2-methoxyphenyl)-6-fluoro-8- methoxy-3-methylimidazo[5,1- c][1,2,4]benzotriazine | 373.16 |
| 69 | 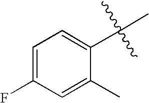 | 6-fluoro-1-(4-fluoro-2-methylphenyl)-8- methoxy-3-methylimidazo[5,1- c][1,2,4]benzotriazine | 341.18 |
| 70 | 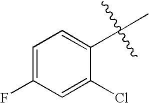 | 1-(2-chloro-4-fluorophenyl)-6-fluoro-8- methoxy-3-methylimidazo[5,1- c][1,2,4]benzotriazine | 361.15 |
| 71 | 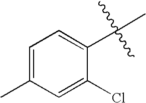 | 1-(2-chloro-4-methylphenyl)-6-fluoro-8- methoxy-3-methylimidazo[5,1- c][1,2,4]benzotriazine | 357.14 |
| 72 | 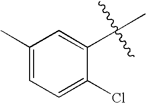 | 1-(2-chloro-5-methylphenyl)-6-fluoro-8- methoxy-3-methylimidazo[5,1- c][1,2,4]benzotriazine | 357.14 |
| 73 | 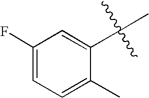 | 6-fluoro-1-(5-fluoro-2-methylphenyl)-8- methoxy-3-methylimidazo[5,1- c][1,2,4]benzotriazine | 341.12 |
| 74 | 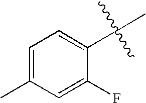 | 6-fluoro-1-(2-fluoro-4-methylphenyl)-8- methoxy-3-methylimidazo[5,1- c][1,2,4]benzotriazine | 341.12 |
| 75 | 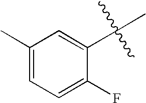 | 6-fluoro-1-(2-fluoro-5-methylphenyl)-8- methoxy-3-methylimidazo[5,1- c][1,2,4]benzotriazine | 341.12 |
| 76 | 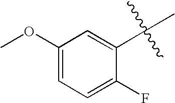 | 6-fluoro-1-(2-fluoro-5-methoxyphenyl)-8- methoxy-3-methylimidazo[5,1- c][1,2,4]benzotriazine | 357.14 |
| 77 | 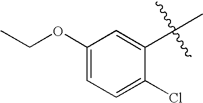 | 1-(2-chloro-5-ethoxyphenyl)-6-fluoro-8- methoxy-3-methylimidazo[5,1- c][1,2,4]benzotriazine | 387.15 |
| 78 | 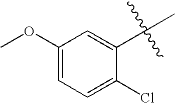 | 1-(2-chloro-5-methoxyphenyl)-6-fluoro-8- methoxy-3-methylimidazo[5,1- c][1,2,4]benzotriazine | 373.1 |
| 79 | 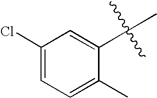 | 1-(5-chloro-2-methylphenyl)-6-fluoro-8- methoxy-3-methylimidazo[5,1- c][1,2,4]benzotriazine | 357.14 |
| 80 | 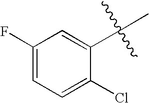 | 1-(2-chloro-5-fluorophenyl)-6-fluoro-8- methoxy-3-methylimidazo[5,1- c][1,2,4]benzotriazine | 361.08 |
| 81 | 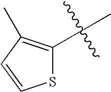 | 6-fluoro-8-methoxy-3-methyl-1-(3- methylthiophen-2-yl)imidazo[5,1- c][1,2,4]benzotriazine | 329.1 |
| 82 | 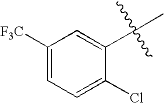 | 1-[2-chloro-5-(trifluoromethyl)phenyl]-6- fluoro-8-methoxy-3-methylimidazo[5,1- c][1,2,4]benzotriazine | 411.05 |
| 83 | 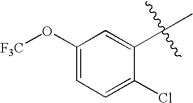 | 1-[2-chloro-5-(trifluoromethoxy)phenyl]-6- fluoro-8-methoxy-3-methylimidazo[5,1- c][1,2,4]benzotriazine | 427.01 |
| 84 | 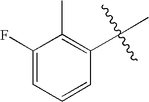 | 6-fluoro-1-(3-fluoro-2-methylphenyl)-8- methoxy-3-methylimidazo[5,1- c][1,2,4]benzotriazine | 341.12 |
| 85 | 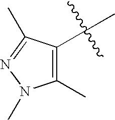 | 6-fluoro-8-methoxy-3-methyl-1-(1,3,5- trimethyl-1H-pyrazol-4-yl)imidazo[5,1- c][1,2,4]benzotriazine | 341.18 |
| 86 | 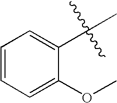 | 6-fluoro-8-methoxy-1-(2-methoxyphenyl)-3- methylimidazo[5,1-c][1,2,4]benzotriazine | 339.15 |
| 87 | 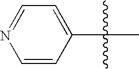 | 6-fluoro-8-methoxy-3-methyl-1-pyridin-4- ylimidazo[5,1-c][1,2,4]benzotriazine | 310.1 |
| 88 | 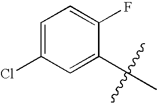 | 1-(5-chloro-2-fluorophenyl)-6-fluoro-8- methoxy-3-methylimidazo[5,1- c][1,2,4]benzotriazine | 361.01 |
| 89 | 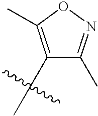 | 1-(3,5-dimethylisoxazol-4-yl)-6-fluoro-8- methoxy-3-methylimidazo[5,1- c][1,2,4]benzotriazine | 328.12 |
| 90 | 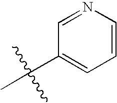 | 6-fluoro-8-methoxy-3-methyl-1-pyridin-3- ylimidazo[5,1-c][1,2,4]benzotriazine | 310.06 |
| 91 | 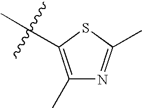 | 1-(2,4-dimethyl-1,3-thiazol-5-yl)-6-fluoro-8- methoxy-3-methylimidazo[5,1- c][1,2,4]benzotriazine | 344.07 |
| 92 | 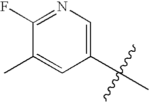 | 6-fluoro-1-(6-fluoro-5-methylpyridin-3-yl)-8- methoxy-3-methylimidazo[5,1- c][1,2,4]benzotriazine | 342.1 |
| 93 | 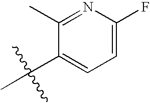 | 6-fluoro-1-(6-fluoro-2-methylpyridin-3-yl)-8- methoxy-3-methylimidazo[5,1- c][1,2,4]benzotriazine | 342.1 |
| 94 | 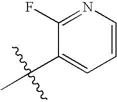 | 6-fluoro-1-(2-fluoropyridin-3-yl)-8-methoxy- 3-methylimidazo[5,1-c][1,2,4]benzotriazine | 328.09 |
| 95 | 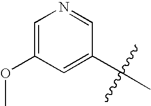 | 6-fluoro-8-methoxy-1-(5-methoxypyridin-3- yl)-3-methylimidazo[5,1- c][1,2,4]benzotriazine | 340.11 |
| 96 | 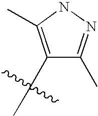 | 1-(3,5-dimethyl-1H-pyrazol-4-yl)-6-fluoro-8- methoxy-3-methylimidazo[5,1- c][1,2,4]benzotriazine | 327.13 |
| 97 | 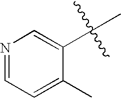 | 6-fluoro-8-methoxy-3-methyl-1-(4- methylpyridin-3-yl)imidazo[5,1- c][1,2,4]benzotriazine | 324.12 |
| 98 | 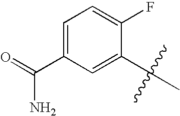 | 4-fluoro-3-(6-fluoro-8-methoxy-3- methylimidazo[5,1-c][1,2,4]benzotriazin-1- yl)benzamide | 370.1 |

### Examples 99-131

The examples in Table 6 were prepared according to procedure described above for Examples 67-98 by replacing 3,5-difluoro-4-nitrophenol with 3,5-difluoro-2-nitrophenol (prepared as described in step 1 of Examples 67-98) in Step 2.

TABLE 6

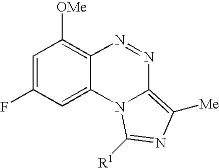

| Example | | | |
|---|---|---|---|
| # | R¹ | Name | MS [M + H]⁺ |
| 99 | 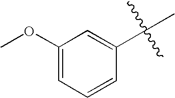 | 8-fluoro-6-methoxy-1-(3-methoxyphenyl)-3- methylimidazo[5,1-c][1,2,4]benzotriazine | 339.22 |
| 100 | 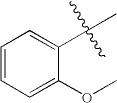 | 8-fluoro-6-methoxy-1-(2-methoxyphenyl)-3- methylimidazo[5,1-c][1,2,4]benzotriazine | 339.22 |
| 101 | 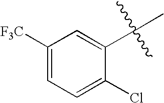 | 1-[2-chloro-5-(trifluoromethyl)phenyl]-8- fluoro-6-methoxy-3-methylimidazo[5,1- c][1,2,4]benzotriazine | 411.12 |
| 102 | 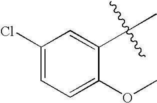 | 1-(5-chloro-2-methoxyphenyl)-8-fluoro-6- methoxy-3-methylimidazo[5,1- c][1,2,4]benzotriazine | 373.16 |
| 103 | 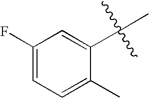 | 8-fluoro-1-(5-fluoro-2-methylphenyl)-6- methoxy-3-methylimidazo[5,1- c][1,2,4]benzotriazine | 341.18 |
| 104 | 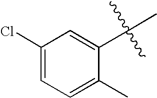 | 1-(5-chloro-2-methylphenyl)-8-fluoro-6- methoxy-3-methylimidazo[5,1- c][1,2,4]benzotriazine | 357.14 |
| 105 | 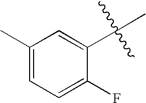 | 8-fluoro-1-(2-fluoro-5-methylpehnyl)-6- methoxy-3-methylimidazo[5,1- c][1,2,4]benzotriazine | 341.18 |
| 106 | 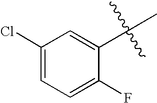 | 1-(5-chloro-2-fluorophenyl)-8-fluoro-6- methoxy-3-methylimidazo[5,1- c][1,2,4]benzotriazine | 361.15 |
| 107 | 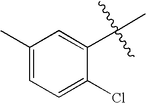 | 1-(2-chloro-5-methylphenyl)-8-fluoro-6- methoxy-3-methylimidazo[5,1- c][1,2,4]benzotriazine | 357.14 |
| 108 | 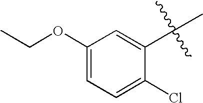 | 1-(2-chloro-5-ethoxyphenyl)-8-fluoro-6- methoxy-3-methylimidazo[5,1- c][1,2,4]benzotriazine | 387.15 |
| 109 | 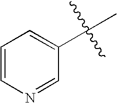 | 8-fluoro-6-methoxy-3-methyl-1-pyridin-3- ylimidazo[5,1-c][1,2,4]benzotriazine | 310.13 |
| 110 | 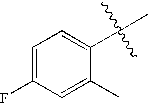 | 8-fluoro-1-(4-fluoro-2-methylphenyl)-6- methoxy-3-methylimidazo[5,1- c][1,2,4]benzotriazine | 341.18 |
| 111 | 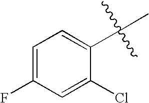 | 1-(2-chloro-4-fluorophenyl)-8-fluoro-6- methoxy-3-methylimidazo[5,1- c][1,2,4]benzotriazine | 361.08 |
| 112 | 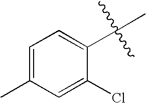 | 1-(2-chloro-4-methylphenyl)-8-fluoro-6- methoxy-3-methylimidazo[5,1- c][1,2,4]benzotriazine | 357.14 |
| 113 | 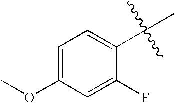 | 8-fluoro-1-(2-fluoro-5-methoxyphenyl)-6- methoxy-3-methylimidazo[5,1- c][1,2,4]benzotriazine | 357.14 |
| 114 | 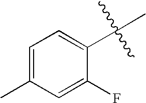 | 8-fluoro-1-(2-fluoro-4-methylphenyl)-6- methoxy-3-methylimidazo[5,1- c][1,2,4]benzotriazine | 341.18 |
| 115 | 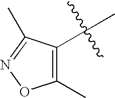 | 1-(3,5-dimethylisoxazol-4-yl)-8-fluoro-6- methoxy-3-methylimidazo[5,1- c][1,2,4]benzotriazine | 328.18 |
| 116 | 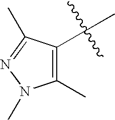 | 8-fluoro-6-methoxy-3-methyl-1-(1,3,5- trimethyl-1H-pyrazol-4-yl)imidazo[5,1- c][1,2,4]benzotriazine | 341.18 |
| 117 | 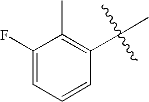 | 8-fluoro-1-(3-fluoro-2-methylphenyl)-6- methoxy-3-methylimidazo[5,1- c][1,2,4]benzotriazine | 341.18 |
| 118 | 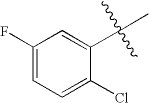 | 1-(2-chloro-5-fluorophenyl)-8-fluoro-6- methoxy-3-methylimidazo[5,1- c][1,2,4]benzotriazine | 361.08 |
| 119 | 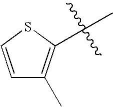 | 8-fluoro-6-methoxy-3-methyl-1-(3- methylthiophen-2-yl)imidazo[5,1- c][1,2,4]benzotriazine | 329.1 |
| 120 | 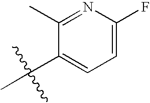 | 8-fluoro-1-(6-fluoro-2-methylpyridin-3-yl)- 6-methoxy-3-methylimidazo[5,1- c][1,2,4]benzotriazine | 342.1 |
| 121 | 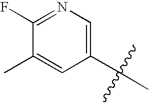 | 8-fluoro-1-(6-fluoro-5-methylpyridin-3-yl)- 6-methoxy-3-methylimidazo[5,1- c][1,2,4]benzotriazine | 342.1 |
| 122 | 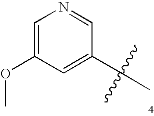 | 8-fluoro-6-methoxy-1-(5-methoxypyridin-3- yl)-3-methylimidazo[5,1- c][1,2,4]benzotriazine | 340.13 |
| 123 | 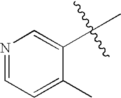 | 8-fluoro-6-methoxy-3-methyl-1-(4- methylpyridin-3-yl)imidazo[5,1- c][1,2,4]benzotriazine | 324.11 |
| 124 | 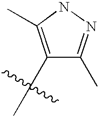 | 1-(3,5-dimethyl-1H-pyrazol-4-yl)-8-fluoro- 6-methoxy-3-methylimidazo[5,1- c][1,2,4]benzotriazine | 327.13 |
| 125 | 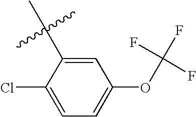 | 1-[2-chloro-5-(trifluoromethoxy)phenyl]-8- fluoro-6-methoxy-3-methylimidazo[5,1- c][1,2,4]benzotriazine | 427.07 |
| 126 | 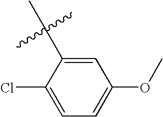 | 1-(2-chloro-5-methoxyphenyl)-8-fluoro-6- methoxy-3-methylimidazo[5,1- c][1,2,4]benzotriazine | 373.1 |
| 127 | 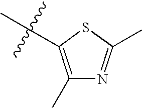 | 1-(2,4-dimethyl-1,3-thiazol-5-yl)-8-fluoro-6- methoxy-3-methylimidazo[5,1- c][1,2,4]benzotriazine | 344.14 |
| 128 | 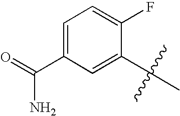 | 4-fluoro-3-(8-fluoro-6-methoxy-3- methylimidazo[5,1-c][1,2,4]benzotriazin-1- yl)benzamide | 370.14 |
| 129 | 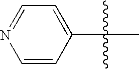 | 8-fluoro-6-methoxy-3-methyl-1-pyridin-4- ylimidazo[5,1-c][1,2,4]benzotriazine | 310.09 |
| 130 | 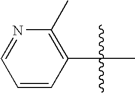 | 8-fluoro-6-methoxy-3-methyl-1-(2- methylpyridin-3-yl)imidazo[5,1- c][1,2,4]benzotriazine | 323.1 |
| 131 | 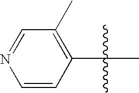 | 8-fluoro-6-methoxy-3-methyl-1-(3- methylpyridin-4-yl)imidazo[5,1- c][1,2,4]benzotriazine | 323.1 |

### Example 132

### 6-Chloro-3-methyl-1-(3-methylpyridin-4-yl)-8-(trifluoromethyl)imidazo[5,1-c][1,2,4]benzotriazine

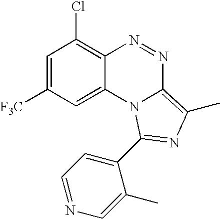

### Step 1: 2-chloro-6-fluoro-4-(trifluoromethyl)aniline

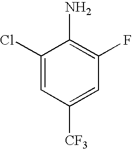

2-Fluoro-4-(trifluoromethyl)aniline (from Matrix Scientific; 5 g, 27.9 mmol) was dissolved in acetonitrile (100 mL). To this was added N-chlorosuccinimide (4 g, 30.7 mmol). The reaction was heated to 75° C. for 16 h then poured into water and extracted with ether. The organic layer was separated and washed with saturated aqueous sodium bicarbonate then water, brine and dried over MgSO₄. The solution was filtered and the solvent removed under reduced pressure. 5.2 g of yellow oil was recovered. MS [(+)ESI] m/z=212.8 [M−H]⁺.

### Step 2: 1-Chloro-3-fluoro-2-nitro-5-(trifluoromethyl)benzene

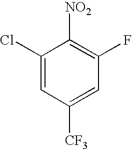

Sodium perborate tetrahydrate (7.3 g, 46.8 mmol) was suspended in glacial acetic acid (30 mL) and heated to 50° C. To this was added dropwise a solution of 2-chloro-6-fluoro-4-(trifluoromethyl)aniline (2 g, 9.37 mmol) dissolved in glacial acetic acid (20 mL). The reaction was stirred for 16 h at 50° C. The reaction was then poured into water and extracted with ether. The organic layer was separated and washed with water, then dilute aqueous bicarbonate solution and brine. The organic layer was then dried over MgSO₄ and filtered. The solvent was removed under reduced pressure and the crude purified by flash chromatography on silica gel in hexane. 1.17 g of a brown oil was recovered. MS [(+)ESI] m/z=242.8 [M−H]⁺.

### Step 3: 1-(3-Chloro-2-nitro-5-(trifluoromethyl)phenyl)-4-methyl-1H-imidazole

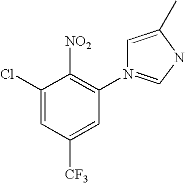

1-Chloro-3-fluoro-2-nitro-5-(trifluoromethyl)benzene (1.1 g, 4.5 mmol) and 4-methyl-1H-imidazole (from Sigma-Aldrich Co.; 0.371 g, 4.5 mmol) were dissolved in DMF (10 mL). To this was added potassium carbonate (1.2 g, 9.0 mmol). The reaction was let stir at RT for 16 h The reaction was poured into water and extracted with ethyl acetate. The organic layer was separated and washed with water then brine and dried over MgSO₄. The solution was then filtered and the solvent removed under reduced pressure. The crude was purified by flash chromatography on silica gel in 10:2 hexane/ethyl acetate. 0.74 g of a tan solid was collected. MS [(+)ESI] m/z=306.0 [M−H]⁺.

### Step 4: 2-Chloro-6-(4-methyl-1H-imidazol-1-yl)-4-(trifluoromethyl)aniline

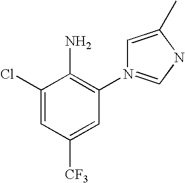

1-(3-Chloro-2-nitro-5-(trifluoromethyl)phenyl)-4-methyl-1H-imidazole (0.74 g, 2.4 mmol) was dissolved in a solution of glacial acetic acid and ethanol (10 mL each). To this was then added iron powder (0.81 g, 14.4 mmol). The reaction was heated to 100° C. for 1 h. The reaction was poured into aqueous sodium hydroxide 1N and extracted with ethyl acetate. The organic layer was separated then washed with water, brine and dried over MgSO₄. The resulting solution was filtered and the solvent removed under reduced pressure. An off white solid (0.68 g) was recovered. MS [(+)ESI] m/z=274.1 [M−H]⁺.

### Step 5: 6-Chloro-3-methyl-8-(trifluoromethyl)benzo[e]imidazo[5,1-c][1,2,4]triazine

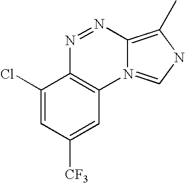

2-Chloro-6-(4-methyl-1H-imidazol-1-yl)-4-(trifluoromethyl)aniline (1 g, 3.6 mmol) was dissolved in glacial acetic acid (20 mL). To this was added sodium nitrite (0.25 g, 3.6 mmol)). The reaction was let stir 2 h at RT then poured into aqueous sodium hydroxide (1N) and extracted with ethyl acetate. The organic layer was separated then washed with water, brine and dried over MgSO₄. The resulting solution was filtered and removed of solvent under reduced pressure. The crude purified by flash chromatography on silica gel in 10:2 hexane/ethyl acetate. 0.4 g of a yellow solid was collected. MS [(+)ESI] m/z=287.0 [M−H]⁺.

### Step 6: 1-Bromo-6-chloro-3-methyl-8-(trifluoromethyl)benzo[e]imidazo[5,1-c][1,2,4]triazine

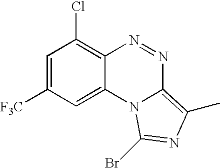

6-Chloro-3-methyl-8-(trifluoromethyl)benzo[e]imidazo[5,1-c][1,2,4]triazine (0.45 g, 1.57 mmol) was dissolved in acetonitrile (20 mL). To this was added N-bromosuccinamide (1.1 g, 6.3 mmol)). The reaction was covered from light and stirred at RT for 48 h. The reaction was poured into water and extracted with chloroform. The organic layer was separated then brine and dried over MgSO₄ and filtered. The solvent was removed under reduced pressure and the crude put on silica gel in 10:1 hexane/ethyl acetate. 0.43 g of a yellow solid was recovered. MS [(+)ESI] m/z=364.9 [M−H]⁺.

### Step 7: 6-Chloro-3-methyl-1-(3-methylpyridin-4-yl)-8-(trifluoromethyl)imidazo[5,1-c][1,2,4]benzotriazine

1-Bromo-6-chloro-3-methyl-8-(trifluoromethyl)benzo[e]imidazo[5,1-c][1,2,4]triazine (0.1 g, 0.27 mmol) and 3-methylpyridin-4-ylboronic acid (from Asymchem Laboratories, Inc.; 0.056 g, 0.41 mmol) were suspended in a solution containing dioxane (4 mL) and water (1 mL). To this was then added potassium carbonate (74 mg, 2 eq). Argon was bubbled thru the reaction for 1 min and Pd(PPh₃)₄ (16 mg, 0.014 mmol) was added. The reaction was sealed and heated to 110° C. for 3 hrs. The reaction was then poured into water and extracted with ethyl acetate. The organic layer was brine then dried over MgSO₄ and filtered. The solvent was removed under reduced pressure and the crude purified by flash chromatography on silica gel in 1:1 hexane/ethyl acetate. (30 mg, 30% yield) of a yellow solid was collected. MS [(+)ESI] m/z=378.0 [M−H]⁺.

### Example 133

### 6-chloro-1-(2,5-dichlorophenyl)-3-methyl-8-(trifluoromethyl)imidazo[5,1-c][1,2,4]benzotriazine

Following the synthetic sequence in Example 132, the titled compound was prepared by replacing the 3-methylpyridin-4-ylboronic acid with 2,5-dichlorophenylboronic acid (from Sigma-Aldrich Co.). MS (ES) m/z 431.0 [M+H]⁺.

### Example 134 (Route 1)

### 6-methoxy-3-methyl-1-(3-methylpyridin-4-yl)-8-(trifluoromethyl)benzo[e]imidazo[5,1-c][1,2,4]triazine

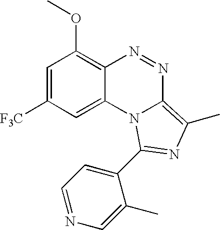

### Step 1: 1,3-difluoro-2-nitro-5-(trifluoromethyl)benzene

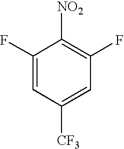

Sodium perborate tetrahydrate (10 g, 4 eq) was suspended in glacial acetic acid (60 mL) and heated to 55° C. 2,6-difluoro-4-(trifluoromethyl)aniline (prepared according to the procedure in European patent application EP315869 A2; 5 g, 25.4 mmol) dissolved in acetic acid (20 mL) was added drop-wise rapidly. The reaction was stirred at 55° C. for 2 h. The reaction mixture was poured into water and extracted with ether. The organic layer was separated and washed with aqueous bicarbonate, water then separated, brine and dried over MgSO₄ and filtered. The solvent was carefully removed (product volatile) under reduced pressure and the crude purified by flash chromatography on silica gel in hexane. 0.198 g (35%, yield) of an orange oil was recovered. MS [(+)ESI] m/z=228.1 [M−H]⁺.

### Step 2: 1-(3-fluoro-2-nitro-5-(trifluoromethyl)phenyl)-4-methyl-1H-imidazole

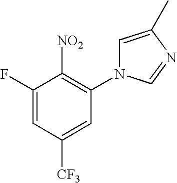

4-methyl-1H-imidazole (2.433 g, 29.6 mmol) and 1,3-difluoro-2-nitro-5-(trifluoromethyl)benzene (6.73 g, 29.6 mmol) were dissolved in DMF (10 mL). To this was added K₂CO₃ (1 g, 2 eq). The reaction was stirred at RT for 4 h. The reaction was poured into water and extracted with ethyl acetate. The organic layer was separated and washed with water, brine and dried over MgSO₄ then filtered. The solvent was removed under reduced pressure and the crude taken in small amount of CHCl₃ and purified by flash chromatography on silica gel 20 to 30% hexane/ethyl acetate. 4.2 g (49% yield) of a pale yellow solid was recovered. MS [(+)ESI] m/z=290.1 [M−H]⁺.

### Step 3: 1-(3-methoxy-2-nitro-5-(trifluoromethyl)phenyl)-4-methyl-1H-imidazole

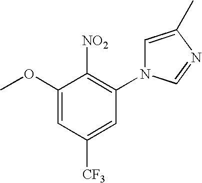

1-(3-fluoro-2-nitro-5-(trifluoromethyl)phenyl)-4-methyl-1H-imidazole (0.2 g, 0.692 mmol) was dissolved in MeOH (3 mL). To this was added NaOMe/MeOH (25%) (2 eq, 0.36 mL). The reaction darkened slightly and was stirred at RT for 2 hrs. The reaction was poured into water and extracted with ethyl acetate. The organic layer was separated and washed with water, brine and dried over MgSO₄, then filtered. The solvent was removed under reduced pressure. 0.18 g of a tan solid (90% yield) was recovered. MS [(+)ESI] m/z=302.1 [M−H]⁺.

### Step 4: 2-methoxy-6-(4-methyl-1H-imidazol-1-yl)-4-(trifluoromethyl)aniline

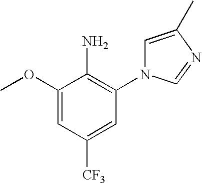

1-(3-methoxy-2-nitro-5-(trifluoromethyl)phenyl)-4-methyl-1H-imidazole (0.18 g, 0.598 mmol) was dissolved in a solution containing ethanol and glacial acetic acid (4 mL each). To this was added iron powder (0.2 g, 6 eq). The reaction was heated to 100° C. for 1 h then poured into aqueous NaOH and extracted with ethyl acetate. The organic layer was separated and washed with water, brine and dried over MgSO₄, then filtered and removed of solvent under reduced pressure. A tan solid 0.15 g (94% yield) was recovered. MS [(+)ESI] m/z=272.1 [M−H]⁺.

### Step 5: 6-methoxy-3-methyl-8-(trifluoromethyl)benzo[e]imidazo[5,1-c][1,2,4]triazine

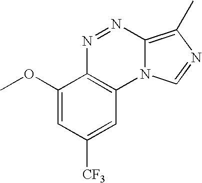

2-methoxy-6-(4-methyl-1H-imidazol-1-yl)-4-(trifluoromethyl)aniline (0.15 g, 0.553 mmol) was dissolved in glacial acetic acid (4 mL). To this was added sodium nitrite (40 mg, 1.05 eq). The reaction turned yellow and was stirred to 1 h at RT then poured into aqueous NaOH and extracted with ethyl acetate. The organic layer was separated and washed with water then brine and dried over MgSO₄ and filtered. The solvent was removed under reduced pressure. 0.11 g (73%) of a yellow solid was recovered. MS [(+)ESI] m/z=283.1 [M−H]⁺.

### Step 6: 1-bromo-6-methoxy-3-methyl-8-(trifluoromethyl)benzo[e]imidazo[5,1-c][1,2,4]triazine

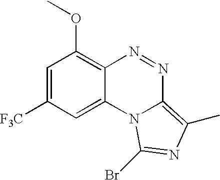

6-methoxy-3-methyl-8-(trifluoromethyl)benzo[e]imidazo[5,1-c][1,2,4]triazine (0.1 g, 0.354 mmol) was dissolved in acetonitrile (5 mL). To this was added N-bromo succinamide (4 eq, 0.25 g). The reaction was protected from light and stirred at RT for 16 h. A yellow solid formed and was filtered and washed with acetonitrile, then dried. 0.1 g (77% yield) of a yellow solid was recovered. MS [(+)ESI] m/z=361.0 [M−H]⁺.

### Step 7: 6-methoxy-3-methyl-1-(3-methylpyridin-4-yl)-8-(trifluoromethyl)benzo[e]imidazo[5,1-c][1,2,4]triazine

1-bromo-6-methoxy-3-methyl-8-(trifluoromethyl)benzo[e]imidazo[5,1-c][1,2,4]triazine (0.1 g, 0.277 mmol) and 3-methylpyridin-4-ylboronic acid (0.076 g, 0.554 mmol) were suspended in a solution containing dioxane (4 mL) and water (1 mL). To this was added K₂CO₃ (2 eq, 74 mg), argon was bubbled thru the reaction for 1 min and Pd(PPh₃)₄ (5% mol, 16 mg) was added. The reaction was sealed and heated to 110° C. for 16 h then diluted with water and extracted with ethyl acetate. The organic layer was separated and dried over MgSO₄, filtered, and the solvent removed under reduced pressure. The crude was purified by flash chromatography on silica gel in 10:2 methylene chloride/ethyl acetate. 30 mg (30% yield) of a yellow solid was recovered. MS [(+)ESI] m/z=374.1 [M−H]⁺. ¹H NMR (400 MHz, d₆-DMSO) δ ppm 8.76 (s, 1H), 8.66 (d, J=5.0 Hz, 1H), 7.60 (d, J=4.8 Hz, 1H), 7.52 (s, 1H), 6.75 (s, 1H), 4.14 (s, 3H), 2.83 (s, 3H), 2.11 (s, 3H).

### Alternate Procedure for Example 134 (Route 2)

### 6-methoxy-3-methyl-1-(3-methylpyridin-4-yl)-8-(trifluoromethyl)benzo[e]imidazo[5,1-c][1,2,4]triazine

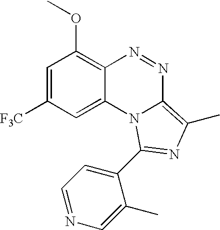

### Step 1: 2-bromo-1-(3-fluoro-2-nitro-5-(trifluoromethyl)phenyl)-4-methyl-1H-imidazole

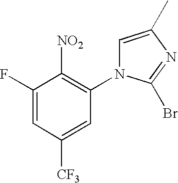

1,3-difluoro-2-nitro-5-(trifluoromethyl)benzene (prepared as described above procedure, Step 1; 1.98 g, 8.72 mmol) and 2-bromo-4-methyl-1H-imidazole (1.404 g, 8.72 mmol) were dissolved in DMF (20 mL). To this was added K₂CO₃ (2 eq, 2.4 g). The reaction was stirred at RT for 1.5 h. The reaction was poured into water and extracted with ethyl acetate. The organic layer was separated and washed with water then brine, dried over MgSO₄ and filtered. The solvent was removed under reduced pressure and the crude was taken in CHCl₃. The resulting precipitate was filtered and collected. The mother liquor was purified by flash chromatography on silica gel in 10:1 to 10:2 hexane/ethyl acetate. 1.76 g (55% yield) of a pale yellow solid was recovered. MS [(+)ESI] m/z=260.0 [M−H]⁺.

### Step 2: 2-bromo-1-(3-methoxy-2-nitro-5-(trifluoromethyl)phenyl)-4-methyl-1H-imidazole

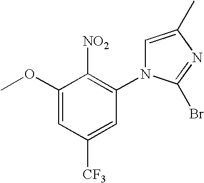

2-bromo-1-(3-fluoro-2-nitro-5-(trifluoromethyl)phenyl)-4-methyl-1H-imidazole (1.3 g, 3.53 mmol) was dissolved in MeOH (15 mL). To this was added 25% NaOMe/MeOH (1.1 mL, 1.2 eq). The reaction was let stir at RT for 1 hr then poured into water and extracted with ethyl acetate. The organic layer was separated and washed with water then brine and dried over MgSO₄, and filtered. The solvent was removed under reduced pressure. 1.25 g (93% yield) of a tan solid was recovered. MS [(+)ESI] m/z=380.0 [M−H]⁺.

### Step 3: 4-(1-(3-methoxy-2-nitro-5-(trifluoromethyl)phenyl)-4-methyl-1H-imidazol-2-yl)-3-methylpyridine

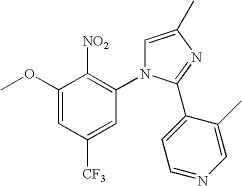

2-bromo-1-(3-methoxy-2-nitro-5-(trifluoromethyl)phenyl)-4-methyl-1H-imidazole (1.25 g, 3.29 mmol) and 3-methylpyridin-4-ylboronic acid (0.901 g, 6.58 mmol) were dissolved in a solution containing dioxane (40 mL) and water (10 mL). To this was then added K₂CO₃ (2 eq, 0.9 g) and argon was bubbled through the reaction for 1 min after which Pd(PPh₃)₄ (0.19 g, 5% mol) was added. The reaction was sealed and heated to 110° C. for 7 h. The reaction was poured into water and extracted with ethyl acetate. The organic layer was separated and brine then dried over MgSO₄ and filtered. The solvent was removed under reduced pressure and the crude purified by flash chromatography on silica gel in 1:1 hexane/ethyl acetate. 0.96 g (73% yield) of a pale yellow solid was recovered. MS [(+)ESI] m/z=393.1 [M−H]⁺.

### Step 4: 2-methoxy-6-(4-methyl-2-(3-methylpyridin-4-yl)-1H-imidazol-1-yl)-4-(trifluoromethyl)aniline

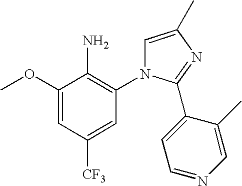

4-(1-(3-methoxy-2-nitro-5-(trifluoromethyl)phenyl)-4-methyl-1H-imidazol-2-yl)-3-methylpyridine (0.9 g, 2.294 mmol) was dissolved in a solution of glacial acetic acid/ethanol (15 mL each). To this was added iron powder (0.7 g, 6 eq). The reaction was heated to 100° C. for 1 hr. The reaction was poured into dilute aqueous NaOH and extracted with ethyl acetate. The organic layer was separated and washed with water then brine, dried over MgSO₄ and filtered. The solvent was removed under reduced pressure. 0.88 g (95% yields) of an off white solid was recovered. MS [(+)ESI] m/z=363.1 [M−H]⁺.

### Step 5: 6-methoxy-3-methyl-1-(3-methylpyridin-4-yl)-8-(trifluoromethyl)benzo[e]imidazo[5,1-c][1, 2, 4]triazine

2-methoxy-6-(4-methyl-2-(3-methylpyridin-4-yl)-1H-imidazol-1-yl)-4-(trifluoromethyl)aniline (0.5 g, 1.380 mmol) was dissolved in glacial acetic acid (8 mL). To this was added sodium nitrite (0.1 g, 1.05 eq). The reaction turned dark clear yellow/orange and was stirred for 0.5 h. The reaction was poured into 1N NaOH and extracted with CHCl₃. The organic layer was separated and washed with water then brine and dried over MgSO₄, filtered, and the solvent removed under reduced pressure. The crude was purified by flash chromatography on silica gel in ethyl acetate. 0.33 g (66% yields) of a yellow solid was recovered. MS [(+)ESI] m/z=374.1 [M−H]⁺. ¹H NMR (400 MHz, d₆-DMSO) δ ppm 8.76 (s, 1H), 8.66 (d, J=5.0 Hz, 1H), 7.60 (d, J=4.8 Hz, 1H), 7.52 (s, 1H), 6.75 (s, 1H), 4.14 (s, 3H), 2.83 (s, 3H), 2.11 (s, 3H).

### Example 135

### 1-(2-Chlorophenyl)-6-methoxy-3-methyl-8-(trifluoromethyl)imidazo[5,1-c][1,2,4]benzotriazine

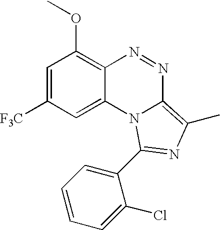

To a suspension of 1-bromo-6-methoxy-3-methyl-8-(trifluoromethyl)benzo[e]imidazo[5,1-c][1,2,4]triazine (prepared according to step 6 of Example 134; 0.100 g, 0.277 mmol) in dioxane (4 mL) was added a solution of potassium carbonate (0.077 g, 2 eq) in water (1 mL). 2-Chlorophenyl boronic acid (from Sigma-Aldrich Co.; 0.065 g, 0.415 mmol) was added, the mixture was degassed and Pd(PPh₃)₄ (0.016 g) was added. The mixture was once again degassed and heated to 110° C. for 3 h (reaction monitored by LC/MS), then cooled to RT, diluted with water and extracted with ethyl acetate. The combined extracts were dried over MgSO₄ and evaporated to dryness under reduced pressure. The crude material was purified by flash chromatography (Isco CombiFlash R_{f}, 40 g Redi-Sep silica gel cartridge, gradient 5-80% EtOAc-hexane, 254 nm detection). Pure fractions were combined and evaporated to dryness to provide the pure title compound as a yellow solid (0.100 g, 92% yield). MS [(+)ESI, m/z]: 393.1 [M+H]⁺. HRMS: Calcd for C₁₈H₁₂ClF₃N₄O+H⁺, 393.0725. Found [(+)ESI, [M+H]⁺]. 393.0732. ¹H NMR (400 MHz, DMSO-d₆): 6 ppm 7.76 (m, 3H), 7.64 (m, 1H), 7.52 (s, 1H), 6.69 (s, 1H), 4.13 (s, 3H), 2.84 (s, 3H).

### Example 136

### 6-Methoxy-3-methyl-1-(2-methylpyridin-3-yl)-8-(trifluoromethyl)imidazo[5,1-c][1,2,4]benzotriazine

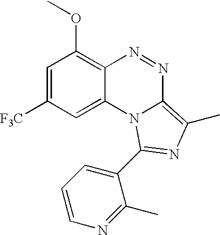

### Step 1. 2-Methyl-3-(4,4,5,5-tetramethyl-[1,3,2]dioxaborolan-2-yl)-pyridine

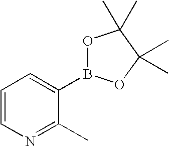

3-Bromo-2-picoline (1 g, 5.8 mmol) was dissolved in dry DMF (20 mL). To this was added potassium acetate (2 g, 3.5 eq), followed by bis(pinacolato)diboron (1.9 g, 1.3 eq). Argon was bubbled through the reaction vessel for 1 min and {1,1-bis(diphenylphosphino)ferrocene} palladium (II)-2CH₂Cl₂ (0.47 g, 10 mole %) was added. The reaction was sealed and heated to 80° C. for 16 h, then cooled to room temperature and poured into water. The aqueous solution was extracted with ethyl acetate and the organic layer separated, washed with brine, dried over magnesium sulfate, filtered and the solvent was removed under reduced pressure. The crude was purified by flash chromatography on silica gel in ethyl acetate. 0.2 g of greenish oil was recovered; MS (ESI) 220.1 [M+1]⁺; ¹H NMR (400 MHz, d₆-DMSO) δ ppm 8.45 (m, 1H), 7.88 (m, 1H), 7.14 (m, 1H), 2.57 (s, 3H), 1.26 (s, 12H).

### Step 2. 6-Methoxy-3-methyl-1-(2-methylpyridin-3-yl)-8-(trifluoromethyl)imidazo[5,1-c][1,2,4]benzotriazine

Prepared according to the procedure of Example 135, using 1-bromo-6-methoxy-3-methyl-8-(trifluoromethyl)benzo[e]imidazo[5,1-c][1,2,4]triazine (0.100 g, 0.277 mmol), potassium carbonate (0.115 g, 3 eq), 2-methyl-3-(4,4,5,5-tetramethyl-1,3,2-dioxaborolan-2-yl)pyridine (0.121 g, 0.554 mmol) and Pd(PPh₃)₄ (0.016 g) in dioxane (4 mL) and water (1 mL). The mixture was stirred at 110° C. for 5 h (monitored by LC/MS). Chromatography of the crude material (Isco, CombiFlash R_{f}, 40 g Redi-Sep silica gel cartridge, gradient 0-10% methanol in 60:40 dichloromethane-EtOAc, 254 nm detection) provided the pure title compound as a yellow solid. (0.070 g, 68% yield). MS [(+)ESI, m/z]: 374.1 [M+H]⁺. HRMS: Calcd for C₁₈H₁₄F₃N₅O+H⁺, 374.12232. Found [(+)ESI, [M+H]⁺], 374.1221. ¹H NMR (400 MHz, DMSO-d₆): 6 ppm 8.76 (m, 1H), 8.0 (d, 1H), 7.51 (s, 1H), 7.48 (d, 1H), 6.65 (s, 1H), 4.13 (s, 3H), 2.83 (s, 3H), 2.24 (s, 3H).

### Example 137

### 6-Methoxy-3-methyl-1-(4-methylpyridin-3-yl)-8-(trifluoromethyl)imidazo[5,1-c][1,2,4]benzotriazine

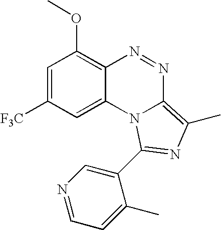

Prepared according to the procedure of Example 135, using 1-bromo-6-methoxy-3-methyl-8-(trifluoromethyl)benzo[e]imidazo[5,1-c][1,2,4]triazine (0.100 g, 0.277 mmol), potassium carbonate (0.115 g, 3 eq), 4-methyl-pyridin-3-yl-boronic acid (from Combi-Blocks Inc.; 0.076 g, 0.554 mmol) and Pd(PPh₃)₄ (0.016 g) in dioxane (4 mL) and water (1 mL). The mixture was stirred at 110° C. for 5 h (monitored by LC/MS), cooled, and additional boronic acid (1 equivalent) added along with Pd(PPh₃)₄ (0.010 g). Heating was resumed for additional 2 h at 110° C. Chromatography of the crude material (Isco, CombiFlash R_{f}, 40 g Redi-Sep silica gel cartridge, gradient of 0-40% EtOAc in dichloromethane followed by a gradient of 0-5% methanol in 60:40 dichloromethane-EtOAc, 254 nm detection) provided 0.079 g of a dark yellow solid. The latter was dissolved in methanol, treated with activated charcoal, and filtered through a plug of Celite. The cake was washed with methanol and ethyl acetate and the golden filtrate evaporated to dryness in vacuo to yield the pure title compound as a yellow solid (0.070 g, 68% yield). ¹H NMR (400 MHz, DMSO-d₆): 6 ppm 8.72 (d, 1H), 8.69 (s, 1H), 7.55 (d, 1H), 7.51 (s, 1H), 6.66 (s, 1H), 4.13 (s, 3H), 2.84 (s, 3H), 2.11 (s, 3H)

### Example 138

### 6-Methoxy-3-methyl-1-(3-methylthiophen-2-yl)-8-(trifluoromethyl)imidazo[5,1-c][1,2,4]benzotriazine

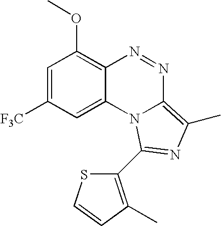

Prepared according to the procedure of Example 135, using 1-bromo-6-methoxy-3-methyl-8-(trifluoromethyl)benzo[e]imidazo[5,1-c][1,2,4]triazine (0.100 g, 0.277 mmol), potassium carbonate (0.115 g, 3 eq), 4,4,5,5-tetramethyl-2-(3-methylthiophen-2-yl)-1,3,2-dioxaborolane (from Sigma-Aldrich Co.; 0.124 g, 0.554 mmol) and Pd(PPh₃)₄ (0.016 g) in dioxane (4 mL) and water (1 mL). The mixture was stirred at 110° C. for 3 h (monitored by LC/MS). Chromatography of crude material (Isco CombiFlash R_{f}, 40 g Redi-Sep silica gel cartridge, gradient 0-60% EtOAc-hexane, 254 nm detection) provided the pure title compound as a yellow solid (0.092 g, 88%). MS [(+)ESI, m/z]: 379.1 [M+H]⁺. HRMS: Calcd for C₁₇H₁₃F₃N₄OS+H⁺, 379.0835. Found [(+)ESI, [M+H]⁺, 379.0839. ¹H NMR (400 MHz, DMSO-d₆): 6 ppm 7.91 (d, 1H), 7.52 (s, 1H), 7.19 (d, 1H), 6.99 (s, 1H), 4.13 (s, 3H), 2.80 (s, 3H), 1.98 (s, 3H)

### Example 139

### 6-Methoxy-1-(3-methoxypyridin-4-yl)-3-methyl-8-(trifluoromethyl)benzo[e]imidazo[5,1-c][1,2,4]triazine

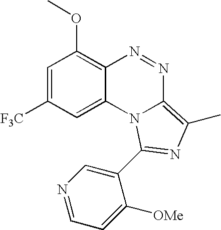

To a suspension of 1-bromo-6-methoxy-3-methyl-8-(trifluoromethyl)benzo[e]imidazo[5,1-c][1,2,4]triazine (prepared according to step 6 of Example 134; 0.110 g, 0.305 mmol) in dioxane (6 mL) and 3 ml of water was added 5-methoxypyridin-3-ylboronic acid (from Combi-Blocks Inc.; 0.058 g, 0.381 mmol), sodium carbonate (0.097 g, 0.914 mmol) and Pd(PPh₃)₄ (0.018 g, 0.015 mmol) in a sealed tube under N₂ cover. The reaction was heated to 110° C. overnight then cooled to RT, diluted with water and extracted with ethyl acetate (2×). The combined extracts were dried over MgSO₄ and evaporated to dryness under reduced pressure. The crude material was purified by flash chromatography (ISCO CombiFlash, 24 g Redi-Sep silica gel cartridge, gradient 5-80% EtOAc-DCM then MeOH-DCM gradient (5-30%). 0.05 g (42%) of the title product (yellow solid) was obtained. MS ((+)ESI, m/z): 389.33 (M+H)⁺. ¹H NMR: (400 MHz, CDCl₃): 6 ppm 8.55 (s, 2H), 7.5 (d, 1H), 7.3 (s, 1H), 7.25 s, 1H), 4.2 (s, 3H), 3.9 (s, 3H), 2.95 (s, 3H)

### Example 140

### 1-(2,5-Dichlorophenyl)-6-methoxy-3-methyl-8-(trifluoromethyl)benzo[e]imidazo[5,1-c][1,2,4]triazine

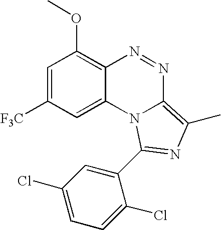

To a suspension of 1-bromo-6-methoxy-3-methyl-8-(trifluoromethyl)benzo[e]imidazo[5,1-c][1,2,4]triazine (prepared according to step 6 of Example 134; 0.13 g, 0.36 mmol) in dioxane (6 mL) and 3 ml of water was added 2,5-dichloroophenylboronic acid (0.086 g, 0.45 mmol), sodium carbonate (0.114 g, 1.08 mmol) and Pd(PPh₃)₄ (0.021 g, 0.018 mmol) in a sealed tube under N₂ cover. The reaction was heated to 110° C. overnight, then cooled to RT, diluted with water and extracted with ethyl acetate (2×). The combined extracts were dried over MgSO₄ and evaporated to dryness under reduced pressure to provide 0.11 g of crude brown solid. The crude material was purified by flash chromatography (ISCO CombiFlash, 24 g Redi-Sep silica gel cartridge, gradient 5-80% EtOAc-DCM). 03 g (19.5%, yellow solid) of the title compound was recovered. MS: ((+)ESI, m/z): 427.21 (M+H)⁺. ¹H NMR: (400 MHz, CDCl₃): 6 ppm 7.69 (s, 1H), 7.55 (m, 3H), 7.15 (s, 1H), 6.95 s. 1H)

### Example 141

### 1-(3-Fluoro-2-methylphenyl)-6-methoxy-3-methyl-8-(trifluoromethyl)benzo[e]imidazo[5,1-c][1,2,4]triazine

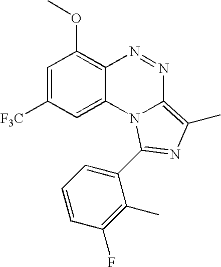

To a suspension of 1-bromo-6-methoxy-3-methyl-8-(trifluoromethyl)benzo[e]imidazo[5,1-c][1,2,4]triazine (prepared according to step 6 of Example 134; 0.120 g, 0.332 mmol) in dioxane (6 mL) and 3 ml of water was added 3-fluoro-2methyl-phenylboronic acid (from Combi-Blocks Inc.; 0.064 g, 0.415 mmol), sodium carbonate (0.106 g, 0.997 mmol) and Pd(PPh₃)₄ (0.019 g, 0.017 mmol) in a sealed tube under N₂ cover. The reaction was heated to 110° C. overnight, then cooled to RT, diluted with water and extracted with ethyl acetate (2×). The combined extracts were dried over MgSO₄ and evaporated to dryness under reduced pressure. The crude material was purified by flash chromatography (ISCO CombiFlash 24 g Redi-Sep silica gel cartridge, gradient 5-80% EtOAc-DCM). 0.06 g (50%) of the title compound was recovered. MS: ((+)ESI, m/z): 390.34 (M+H)⁺. ¹H NMR: (400 MHz, CDCl₃) δ ppm 7.45 (m, 1H), 7.35-7.25 (m, 3H), 6.88 (s, 1H), 4.19 (s, 3H), 4.19 s, 3H), 2.00 (s, 3H)

### Example 142

### 1-(5-Chloro-2-methoxyphenyl)-6-methoxy-3-methyl-8-(trifluoromethyl)benzo[e]imidazo[5,1-c][1,2,4]triazine

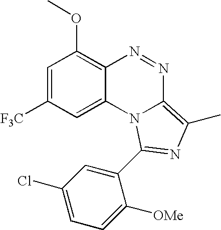

To a suspension of 1-bromo-6-methoxy-3-methyl-8-(trifluoromethyl)benzo[e]imidazo[5,1-c][1,2,4]triazine (prepared according to step 6 of Example 134; 0.110 g, 0.305 mmol) in dioxane (6 mL) and 3 ml of water was added 5-chloro-2-methoxy-phenylboronic acid (from Combi-Blocks Inc.; 0.071 g, 0.381 mmol), sodium carbonate (0.097 g, 0.914 mmol) and Pd(PPh₃)₄ (0.018 g, 0.015 mmol) in a sealed tube under N₂ cover. The reaction was heated to 110° C. for overnight then cooled to RT, diluted with water and extracted with ethyl acetate (2×). The combined extracts were dried over MgSO₄ and evaporated to dryness under reduced pressure. The crude material was purified by flash chromatography (ISCO CombiFlash, 24 g Redi-Sep silica gel cartridge, gradient 10-80% EtOAc-hexane). 0.045 g (35%) of the title compound was recovered. MS: ((+)ESI, m/z):423.1 (M+H)⁺. ¹H NMR: (400 MHz, CDCl₃) δ ppm 7.68 (s, 1H), 7.56 (d, 1H), 7.25 (s, 1H), 7.25 6.88 (d, 1H), 3.58 (s, 3H), 2.94 (s, 3H), 2.92 (s, 3H)

### Example 143

### 1-(Furan-3-yl)-6-methoxy-3-methyl-8-(trifluoromethyl)benzo[e]imidazo[5,1-c][1,2,4]triazine

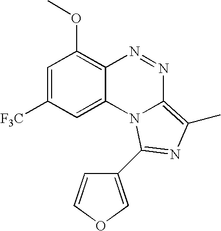

To a suspension of 1-bromo-6-methoxy-3-methyl-8-(trifluoromethyl)benzo[e]imidazo[5,1-c][1,2,4]triazine (prepared according to step 6 of Example 134; 0.120 g, 0.332 mmol) in dioxane (6 mL) and 3 ml of water was added furan-3-yl boronic acid (from Sigma-Aldrich Co.; 0.046 g, 0.415 mmol), sodium carbonate (0.106 g, 0.997 mmol) and Pd(PPh₃)₄ (0.019 g, 0.017 mmol) in a sealed tube under N₂ cover. The reaction was heated to 110° C. overnight then cooled to RT, diluted with water and extracted with ethyl acetate (2×). The combined extracts were dried over MgSO₄ and evaporated to dryness under reduced pressure. The crude material was purified by flash chromatography (ISCO CombiFlash, 24 g Redi-Sep silica gel cartridge, gradient 5-80% EtOAc-hexane). 0.075 g (65%) of the title compound was recovered as a yellow solid. MS: ((+)ESI, m/z): 348.28 (M+H)⁺. ¹H NMR: (400 MHz, CDCl₃) δ ppm 7.97 (s, 1H), 7.7 (s, 1H), 7.68 (s, 1H), 7.25 (d, 1H) 4.19 s, 3H), 2.91 (s, 3H)

### Example 144

### 4-(6-Methoxy-3-methyl-8-(trifluoromethyl)benzo[e]imidazo[5,1-c][1,2,4]triazin-1-yl)-3,5-dimethylisoxazole

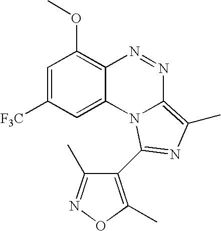

To a suspension of 1-bromo-6-methoxy-3-methyl-8-(trifluoromethyl)benzo[e]imidazo[5,1-c][1,2,4]triazine (prepared according to step 6 of Example 134; 0.110 g, 0.305 mmol) in dioxane (6 mL) and 3 ml of water was added 3,5-dimethylisoxazol-4-ylboronic acid (from Acros Organics; 0.048 g, 0.343 mmol), sodium carbonate (0.097 g, 0.914 mmol) and Pd(PPh₃)₄ (0.018 g, 0.015 mmol) in a sealed tube under N₂ cover. The reaction was heated to 110° C. overnight then cooled to RT, diluted with water and extracted with ethyl acetate (2×). The combined extracts were dried over MgSO₄ filtered and stripped to a brown solid. (0.13 g). The product was charged on an ISCO 24 g cartridge using a EtOAc:DCM gradient 10-80%. followed by a MeOH:DCM (5-30%) gradient. 0.060 g (52%) of the title compound was recovered as a tan solid. MS: ((+)ESI, m/z): 377.32 (M+H)⁺. ¹H NMR: (400 MHz, CDCl₃) δ ppm 7.38 (s, 1H), 7.25 (s, 1H), 4.20 (s, 3H), 2.93 (s, 3H), 2.39 (s, 3H), 2.17 (s, 3H)

### Example 145

### 6-Methoxy-3-methyl-1-(thiophen-2-yl)-8-(trifluoromethyl)benzo[e]imidazo[5,1-c][1,2,4]triazine

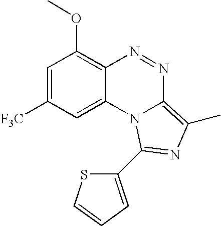

To a suspension of 1-bromo-6-methoxy-3-methyl-8-(trifluoromethyl)benzo[e]imidazo[5,1-c][1,2,4]triazine (prepared according to step 6 of Example 134; 0.110 g, 0.305 mmol) in dioxane (6 mL) and 3 ml of water was added thiophen-2-yl-boronic acid (from Sigma-Aldrich Co.; 0.049 g, 0.381 mmol), sodium carbonate (0.097 g, 0.914 mmol) and Pd(PPh₃)₄ (0.015 g, 0.018 mmol) in a sealed tube under N₂ cover. The reaction was heated to 110° C. overnight then cooled to RT, diluted with water and extracted with ethyl acetate (2×). The combined extracts were dried over MgSO₄ and evaporated to dryness under reduced pressure. The crude material was purified by flash chromatography (ISCO CombiFlash 24 g Redi-Sep silica gel cartridge, gradient 5-80% EtOAc-DCM). 0.075 g (65%) of the title compound was recovered as a yellow solid. MS: ((+)ESI, m/z):364.34 (M+H)⁺. ¹H NMR: (400 MHz, CDCl₃) δ ppm 7.7 (d, 1H), 7.55 (d, 1H), 7.48 (d, 1H), 7.26 (d, 1H), 7.21 (s, 1H), 4.20 (s, 3H), 2.94 (s, 3H)

### Example 146

### 1-(5-Fluoro-2-methylphenyl)-6-methoxy-3-methyl-8-(trifluoromethyl)benzo[e]imidazo[5,1-c][1,2,4]triazine

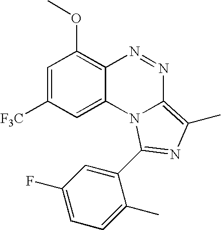

To a suspension of 1-bromo-6-methoxy-3-methyl-8-(trifluoromethyl)benzo[e]imidazo[5,1-c][1,2,4]triazine (prepared according to step 6 of Example 134; 0.0.075 g, 0.208 mmol) in dioxane 6 mL and 3 ml water) was added 5-Fluoro-2-methylphenyl boronic acid (from Sigma-Aldrich Co.; 0.040 g, 0.2601 mmol), sodium carbonate (0.066 g, 0.625 mmol) and Pd(PPh₃)₄ (0.015 g, 0.018 mmol) in a sealed tube under N₂ cover. The reaction was heated to 110° C. for overnight then cooled to RT, diluted with water and extracted with ethyl acetate (2×). The combined extracts were dried over MgSO₄ and evaporated to dryness under reduced pressure. The crude material (0.10) was purified by flash chromatography (ISCO CombiFlash 12 g Redi-Sep silica gel cartridge, gradient 5-80% EtOAc-DCM 0.015 g (18.5%) of the title compound was recovered as a yellow solid. MS: ((+)ESI, m/z): 390.33 (M+H)⁺. ¹H NMR: (400 MHz, CDCl₃) δ ppm 7.40 (m, 1H), 7.25 (m, 1H), 7.21 (m, 2H), 6.89 (s, 1H), 4.19 (s, 3H), 2.96 (s, 3H), 2.02 (s, 3H)

### Example 149

### 6-methoxy-3-methyl-1-(4-methylpyridin-3-yl)benzo[e]imidazo[5,1-c][1,2,4]triazin-8-ol

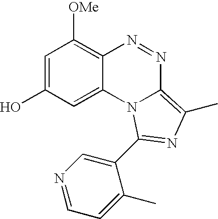

### Step 1: 5-(benzyloxy)-1,3-difluoro-2-nitrobenzene

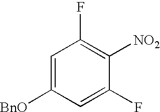

To a mixture of intermediate A1 (14 g, 80 mmol) and potassium carbonate (24.2 g, 176 mmol) in N,N-dimethylformamide (150 mL) was added benzyl bromide (10.45 mL, 88 mmol) at RT. The resulting mixture was stirred at RT overnight. Most solvent was removed under vacuo. The residue was diluted with water and ethyl acetate. Organic layer was separated and the aqueous layer was extracted with ethyl acetate. Combined organic layer was washed with brine and dried over magnesium sulfate. Condensation under vacuo and column chromatography using 20% ethyl acetate in hexane provided the product (98% yield)

### Step 2: 1-(5-(benzyloxy)-3-fluoro-2-nitrophenyl)-2-bromo-4-methyl-1H-imidazole

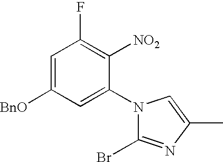

To a mixture of 5-(benzyloxy)-1,3-difluoro-2-nitrobenzene (2.65 g, 10 mmol) and potassium carbonate (2.76 g, 20 mmol) in 50 mL DMF was added 2-bromo-4-methylimidazole (1.46 g, 9.09 mmol) at RT. The resulting mixture was stirred at RT for 40 h. Majority of solvent was removed by rotavap. The residue was diluted with ethyl acetate and washed with water. Extraction with ethyl acetate, condensation on rotavap, followed by column chromatography using 20-40% ethyl acetate in hexane as eluent to provide 1.72 g (47% yield) product as a thick foam.

### Step 3: 1-(5-(benzyloxy)-3-methoxy-2-nitrophenyl)-2-bromo-4-methyl-1H-imidazole

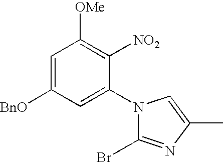

To a solution of 1-(5-(benzyloxy)-3-fluoro-2-nitrophenyl)-2-bromo-4-methyl-1H-imidazole (1.64 g, 4.04 mmol) in 20 mL of MeOH was added freshly powdered KOH (1.1 g, 19.4 mmol) at RT under nitrogen. The resulting mixture was stirred at 55° C. for 2 h, then cooled to RT and diluted with water, extracted with ethyl acetate (3×). Standard work-up followed by column chromatography using 5-10% ethyl acetate in DCM as eluting solvent provided the product (1.65 g, 98% yield) as a yellow powder.

### Step 4: 3-(1-(5-(benzyloxy)-3-methoxy-2-nitrophenyl)-4-methyl-1H-imidazol-2-yl)-4-methylpyridine

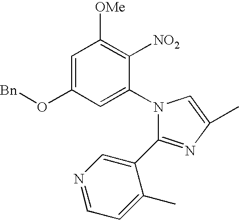

Following the Suzuki coupling step 3 of Example 45, titled compound was prepared from 1-(5-(benzyloxy)-3-methoxy-2-nitrophenyl)-2-bromo-4-methyl-1H-imidazole in 82% yield. EIMS 431.2 [M+1]⁺.

### Step 5: 4-amino-3-methoxy-5-(4-methyl-2-(4-methylpyridin-3-yl)-1H-imidazol-1-yl)phenol

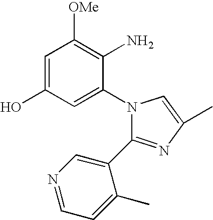

A mixture of 3-(1-(5-(benzyloxy)-3-methoxy-2-nitrophenyl)-4-methyl-1H-imidazol-2-yl)-4-methylpyridine (420 mg, 0.976 mmol) and palladium on carbon (104 mg, 0.098 mmol) was vacuumed and refilled with nitrogen, followed by addition of THF (10 ml) and MeOH (10.00 ml). The mixture was stirred at 60° C. for 2 h, cooled to RT, filtered through Celite, washed with ethyl acetate (3×). Evaporated to provide off-white solid 4-amino-3-methoxy-5-(4-methyl-2-(4-methylpyridin-3-yl)-1H-imidazol-1-yl)phenol (300 mg, 0.967 mmol, 99%). EIMS 311.2 [M+1]⁺.

### Step 6: 6-methoxy-3-methyl-1-(4-methylpyridin-3-yl)benzo[e]imidazo[5,1-e][1,2,4]triazin-8-ol

The titled compound was prepared following step 5 of Example 45, from 4-amino-3-methoxy-5-(4-methyl-2-(4-methylpyridin-3-yl)-1H-imidazol-1-yl)phenol in 26% yield. EIMS 322.2 [M+1]⁺.

### Example 150

### 8-(difluoromethoxy)-6-methoxy-3-methyl-1-(4-methylpyridin-3-yl)benzo[e]imidazo[5,1-c][1,2,4]triazine

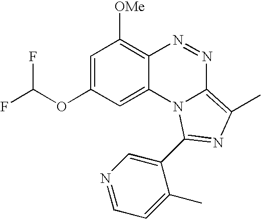

A mixture of 6-methoxy-3-methyl-1-(4-methylpyridin-3-yl)benzo[e]imidazo[5,1-c][1,2,4]triazin-8-ol (Example 149, 70 mg, 0.218 mmol), cesium carbonate (355 mg, 1.089 mmol) and sodium 2-chloro-2,2-difluoroacetate (from TCI, Japan; 166 mg, 1.089 mmol) in a flask was vacuumed and refilled with nitrogen, followed by addition of DMF (5.4 ml) and water (0.600 ml). The resulting mixture was stirred at 100° C. for 5 h. Cooled to RT, diluted with water, extracted with ethyl acetate (3×). Column purification (50-100% EtOAc in DCM, then 3-6% MeOH in DCM) provided the product 8-(difluoromethoxy)-6-methoxy-3-methyl-1-(4-methylpyridin-3-yl)benzo[e]imidazo[5,1-c][1,2,4]triazine (E100519-35-1, 48 mg, 0.129 mmol, 59.3%). EIMS 372.1 [M+1]⁺. ¹H NMR (400 MHz, d₆-DMSO) δ ppm 8.70 (d, J=5.1 Hz, 1H), 8.64 (s, 1H), 7.51 (d, J=5.1 Hz, 1H), 7.11 (t, J=72.7 Hz, 1H), 7.06 (d, J=2.2 Hz, 1H), 6.12 (d, J=2.2 Hz, 1H), 4.05 (s, 3H), 2.80 (s, 3H), 2.08 (s, 3H).

### Example 151

### 8-(benzyloxy)-6-methoxy-3-methyl-1-(3-methylpyridin-4-yl)imidazo[5,1-c][1,2,4]benzotriazine

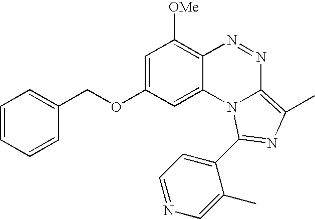

### Step 1: 4-(1-(5-(benzyloxy)-3-methoxy-2-nitrophenyl)-4-methyl-1H-imidazol-2-yl)-3-methylpyridine

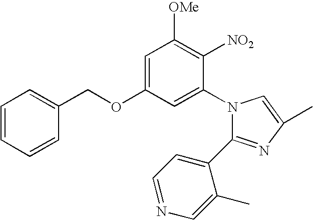

The titled compound was prepared in 80% yield as described in step 4 of Example 149 by replacing the 2-methylpyridin-3-ylboronic acid with 3-methylpyridin-4-ylboronic acid. MS (ESI) 431.2 [M+1]⁺.

### Step 2: 4-(benzyloxy)-2-methoxy-6-(4-methyl-2-(3-methylpyridin-4-yl)-1H-imidazol-1-yl)aniline

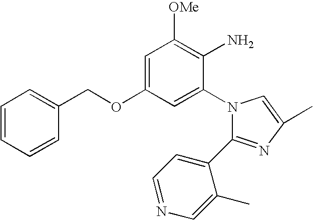

A mixture of 4-(1-(5-(benzyloxy)-3-methoxy-2-nitrophenyl)-4-methyl-1H-imidazol-2-yl)-3-methylpyridine (412 mg, 0.957 mmol) and tin(II)chloride dihydrate (1080 mg, 4.79 mmol) in a 250 mL flask was vacuumed and refilled with nitrogen, followed by the addition of ethanol (10 mL) and hydrochloric acid (37%, 0.286 mL). The final mixture was refluxed at 100° C. for 2 h, cooled to RT. The mixture was poured into cold 1N NaOH solution, extracted with ethyl acetate (3×). Standard work-up followed by column chromatography (50-100% ethyl acetate in DCM, followed by 3-8% MeOH in DCM) provided the product 4-(benzyloxy)-2-methoxy-6-(4-methyl-2-(3-methylpyridin-4-yl)-1H-imidazol-1-yl)aniline (318 mg, 0.794 mmol, 83%). MS (ESI) 401.2 [M+1]⁺.

### Step 3: 8-(benzyloxy)-6-methoxy-3-methyl-1-(3-methylpyridin-4-yl)imidazo[5,1-e][1,2,4]benzotriazine

To a solution of 4-(benzyloxy)-2-methoxy-6-(4-methyl-2-(3-methylpyridin-4-yl)-1H-imidazol-1-yl)aniline (318 mg, 0.794 mmol) in acetic acid (12 ml) was added a solution of sodium nitrite (82 mg, 1.191 mmol) in water (2.00 ml) at RT. The resulting mixture was stirred at RT for 1 h. Solvent was removed by rotavap. The residue was partitioned between ethyl acetate and sodium carbonate solution. Extracted with ethyl acetate (3×). Washed with brine. Column purification (50-100% EtOAc in DCM, then 5-10% MeOH in DCM) provided the product 8-(benzyloxy)-6-methoxy-3-methyl-1-(3-methylpyridin-4-yl)benzo[e]imidazo[5,1-c][1,2,4]triazine (315 mg, 0.766 mmol, 96%) as a yellow powder. MS (ESI) 412.2 [M+1]⁺.

### Example 152

### 6-methoxy-3-methyl-1-(3-methylpyridin-4-yl)benzo[e]imidazo[5,1-c][1,2,4]triazin-8-ol

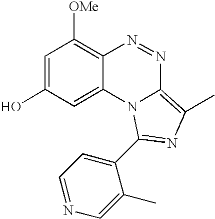

A mixture of 8-(benzyloxy)-6-methoxy-3-methyl-1-(3-methylpyridin-4-yl)benzo[e]imidazo[5,1-c][1,2,4]triazine (315 mg, 0.766 mmol) and palladium on carbon (40.7 mg, 0.038 mmol) in a flask was vacuumed and refilled with nitrogen, followed by addition of THF (8 ml) and MeOH (8.00 ml), then ammonium formate (145 mg, 2.297 mmol) was added in one portion. The final mixture was stirred at 50° C. for 2 h. Cooled to RT. Filtered through Celite, extensively washed with ethyl acetate/MeOH until the filtrate becomes colorless. Solvent was removed by rotavap. The residue was purified by column (3-6-10% MeOH in DCM) provided the product 6-methoxy-3-methyl-1-(3-methylpyridin-4-yl)benzo[e]imidazo[5,1-c][1,2,4]triazin-8-ol (180 mg, 0.560 mmol, 73.2%). MS (ESI) 322.1 [M+1]⁺.

### Example 153

### 8-(difluoromethoxy)-6-methoxy-3-methyl-1-(3-methylpyridin-4-yl)imidazo[5,1-c][1,2,4]benzotriazine

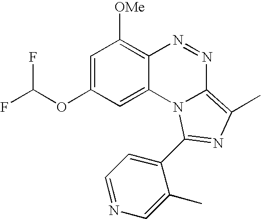

A mixture of 6-methoxy-3-methyl-1-(3-methylpyridin-4-yl)benzo[e]imidazo[5,1-c][1,2,4]triazin-8-ol (80 mg, 0.249 mmol) and potassium carbonate (41.3 mg, 0.299 mmol) in a vial was vacuumed and refilled with nitrogen, followed by addition of DMF (5 ml) and water (0.500 ml). The resulting mixture was warmed to 80° C., then sodium 2-chloro-2,2-difluoroacetate (76 mg, 0.498 mmol) was added quickly in one portion. The final mixture was warmed to 130° C. and stirred at this temperature for 2 h. Cooled to RT. Diluted with water and ethyl acetate. Extracted with ethyl acetate (2×), washed with brine, dried over magnesium sulfate. Column purification (50-100% EtOAc in DCM) provided the product 8-(difluoromethoxy)-6-methoxy-3-methyl-1-(3-methylpyridin-4-yl)benzo[e]imidazo[5,1-c][1,2,4]triazine (52 mg, 0.140 mmol, 56.2%) as a yellow powder. MS (ESI) 372.1 [M+1]⁺. ¹H NMR (400 MHz, d₆-DMSO) δ ppm 8.70 (s, 1H), 8.60 (d, J=4.8 Hz, 1H), 7.53 (d, J=4.8 Hz, 1H), 7.10 (t, J=72.7 Hz, 1H), 7.05 (d, J=2.3 Hz, 1H), 6.19 (d, J=2.3 Hz, 1H), 4.05 (s, 3H), 2.78 (s, 3H), 2.06 (s, 3H).

### Example 154

### 8-(benzyloxy)-6-methoxy-3-methyl-1-(2-methylpyridin-3-yl)imidazo[5,1-c][1,2,4]benzotriazine

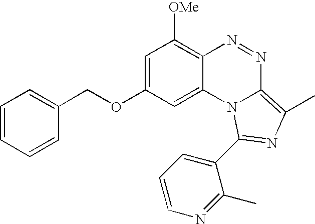

The titled compound was prepared following the procedure described for Example 151 by replacing the 3-methylpyridin-4-ylboronic acid with 2-methylpyridin-3-ylboronic acid. MS (ESI) 412.2 [M+1]⁺.

### Example 155

### 6-methoxy-3-methyl-1-(2-methylpyridin-3-yl)imidazo[5,1-c][1,2,4]benzotriazin-8-ol

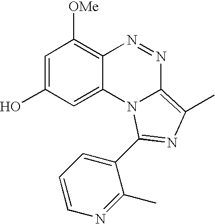

The titled compound was prepared following the procedure described for Example 152. MS (ESI) 322.1 [M+1]⁺.

### Example 156

### 8-(difluoromethoxy)-6-methoxy-3-methyl-1-(2-methylpyridin-3-yl)imidazo[5,1-c][1,2,4]benzotriazine

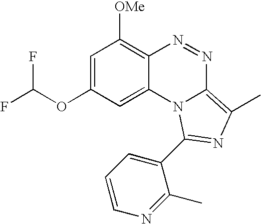

The titled compound was prepared following the procedure described for Example 153. MS (ESI) 372.1 [M+1]⁺. ¹H NMR (400 MHz, d₆-DMSO) δ ppm 8.71 (m, 1H), 7.92 (m, 1H), 7.44 (m, 1H), 7.09 (t, J=72.7 Hz, 1H), 7.04 (d, J=2.3 Hz, 1H), 6.10 (d, J=2.3 Hz, 1H), 4.05 (s, 3H), 2.78 (s, 3H), 2.22 (s, 3H).

### Example 157

### 8-(benzyloxy)-6-methoxy-3-methyl-1-(4-methylpyridin-3-yl)imidazo[5,1-c][1,2,4]benzotriazine

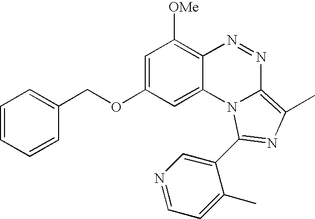

The titled compound was prepared following the procedure described for Example 151 by replacing the 3-methylpyridin-4-ylboronic acid with 4-methylpyridin-3-ylboronic acid. MS (ESI) 412.1 [M+1]⁺.

### Example 158

### 8-(cyclopropylmethoxy)-6-methoxy-3-methyl-1-(3-methylpyridin-4-yl)benzo[e]imidazo[5,1-c][1,2,4]triazine

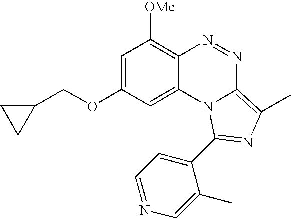

To a mixture of 6-methoxy-3-methyl-1-(3-methylpyridin-4-yl)benzo[e]imidazo[5,1-c][1,2,4]triazin-8-ol (Example 152; 54 mg, 0.168 mmol) and cesium carbonate (82 mg, 0.252 mmol) in 3 mL of DMF was added a solution of (bromomethyl)cyclopropane (from Matrix Scientific; 34.0 mg, 0.252 mmol) in 1 mL of DMF. The resulting mixture was stirred at 100° C. overnight. Cooled to RT. Diluted with water and ethyl acetate. Extracted with ethyl acetate (2×). Washed with brine, dried over magnesium sulfate. Column purification (50-100% EtOAc in DCM) provided the product (35 mg, 0.093 mmol, 55.5%) as a yellow powder. MS (ESI) 376.1 [M+1]⁺. ¹H NMR (400 MHz, d₆-DMSO) δ ppm 8.70 (s, 1H), 8.61 (d, J=5.0 Hz, 1H), 7.54 (d, J=4.8 Hz, 1H), 6.78 (d, J=2.3 Hz, 1H), 5.90 (d, J=2.3 Hz, 1H), 4.01 (s, 3H), 3.50 (d, J=7.0 Hz, 2H), 2.75 (s, 3H), 2.06 (s, 3H), 1.02 (m, 1H), 0.51 (m, 2H), 0.18 (m, 2H).

### Example 159

### 8-(cyclopropylmethoxy)-6-methoxy-3-methyl-1-(2-methylpyridin-3-yl)benzo[e]imidazo[5,1-c][1,2,4]triazine

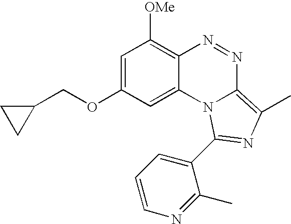

To a mixture of 6-methoxy-3-methyl-1-(2-methylpyridin-3-yl)benzo[e]imidazo[5,1-c][1,2,4]triazin-8-ol (Example 155; 30 mg, 0.093 mmol) and cesium carbonate (45.6 mg, 0.140 mmol) in 3 mL of DMF was added a solution of (bromomethyl)cyclopropane (18.91 mg, 0.140 mmol) in 1 mL of DMF. The resulting mixture was stirred at 100° C. overnight. Cooled to RT. Diluted with water and ethyl acetate. Extracted with ethyl acetate (2×). Washed with brine, dried over magnesium sulfate. Column purification (50-100% EtOAc in DCM) provided the product (28 mg, 0.075 mmol, 80%) as a yellow powder. MS (ESI) 376.1 [M+1]⁺. ¹H NMR (400 MHz, d₆-DMSO) δ ppm 8.70 (m, 1H), 7.95 (m, 1H), 7.46 (m, 1H), 6.77 (d, J=2.3 Hz, 1H), 5.84 (d, J=2.3 Hz, 1H), 4.00 (s, 3H), 3.48 (d, J=7.0 Hz, 2H), 2.75 (s, 3H), 2.20 (s, 3H), 1.00 (m, 1H), 0.51 (m, 2H), 0.20 (m, 2H).

### Examples 160-169

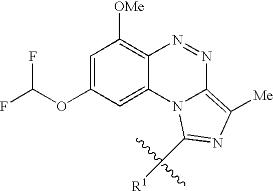

### Step 1: 5-(difluoromethoxy)-1,3-difluoro-2-nitrobenzene

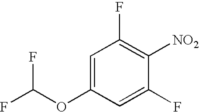

To a solution of 3,5-difluoro-4-nitrophenol (from step 1, Examples 67-98; 500 mg, 2.86 mmol) in acetonitrile (10 ml) was added a solution of potassium hydroxide (3365 mg, 60.0 mmol) in water (10.00 ml). To the resulting mixture was added 2-chloro-2,2-difluoro-1-phenylethanone (from VWR; 2721 mg, 14.28 mmol) at −78° C. The final mixture was warmed to RT, and then 80° C. overnight. Cooled to RT. diluted with water, extracted with ethyl acetate. Column purification (5-10-20% EtOAc in Hexane) provided the product 5-(difluoromethoxy)-1,3-difluoro-2-nitrobenzene (280 mg, 1.244 mmol, 43.6%) as a light brown oil. MS (EI) 225.0 [M]⁺.

### Step 2: 1-(5-(difluoromethoxy)-3-fluoro-2-nitrophenyl)-4-methyl-1H-imidazole

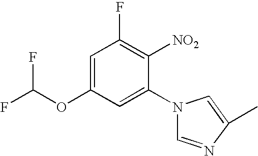

A mixture of 5-(difluoromethoxy)-1,3-difluoro-2-nitrobenzene (2 g, 8.89 mmol), 4-methyl-1H-imidazole (0.729 g, 8.89 mmol) and potassium carbonate (2.456 g, 17.77 mmol) in DMF (80 ml) was stirred at 23° C. overnight. Diluted with water and ethyl acetate. Extracted with ethyl acetate (3×). Column purification (10-30% EtOAc in Hexane) provided the product 1-(5-(difluoromethoxy)-3-fluoro-2-nitrophenyl)-4-methyl-1H-imidazole (1.7 g, 66% yield). MS (ESI) 288.1 [M+1]⁺.

### Step 3: 1-(5-(difluoromethoxy)-3-methoxy-2-nitrophenyl)-4-methyl-1H-imidazole

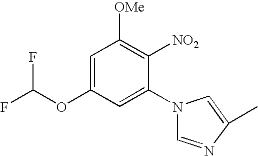

To a solution of 1-(5-(difluoromethoxy)-3-fluoro-2-nitrophenyl)-4-methyl-1H-imidazole (1.7 g, 5.92 mmol) in MeOH (30 ml) was added freshly powdered potassium hydroxide (1.661 g, 29.6 mmol). the resulting mixture was stirred at 55° C. for 2 h. Cooled to RT. Solvent removed by rotavap, diluted with water and ethyl acetate, extracted with ethyl acetate (3×). Column purification (20-40% EtOAc in Hexane) provided the product 1-(5-(difluoromethoxy)-3-methoxy-2-nitrophenyl)-4-methyl-1H-imidazole (800 mg, 2.67 mmol, 45.2%). MS (ESI) 300.1 [M+1]⁺.

### Step 4: 4-(difluoromethoxy)-2-methoxy-6-(4-methyl-1H-imidazol-1-yl)aniline

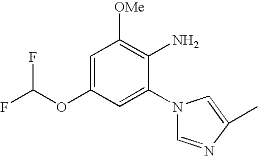

To a mixture of 1-(5-(difluoromethoxy)-3-methoxy-2-nitrophenyl)-4-methyl-1H-imidazole (790 mg, 2.64 mmol) and palladium on carbon (140 mg, 0.132 mmol) was added THF (25 ml) and MeOH (25.00 ml) at RT under nitrogen, followed by addition of ammonium formate (499 mg, 7.92 mmol). The mixture was stirred at 50° C. for 1 h, cooled to RT. filtered through Celite, washed with ethyl acetate. Solvent was removed on rotavap to provide product (710 mg, 2.64 mmol, 100%) as a pale oil. MS (ESI) 270.1 [M+1]⁺.

### Step 5: 8-(difluoromethoxy)-6-methoxy-3-methylbenzo[e]imidazo[5,1-c][1,2,4]triazine

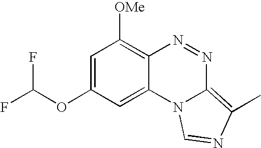

To a solution of 4-(difluoromethoxy)-2-methoxy-6-(4-methyl-1H-imidazol-1-yl)aniline (710 mg, 2.64 mmol) in acetic acid (30 ml) was added a solution of sodium nitrite (280 mg, 4.06 mmol) in water (5.00 ml). The resulting mixture was stirred at RT for 2 h. Solvent was removed by rotavap. The residue was partitioned between sodium carbonate solution and ethyl acetate. Organic phase was isolated and dried. Column purification (50-100% EtOAc in DCM) provided the product. The aqueous phase was evaporated under vacuum to dryness. MeOH was added to extract the product. Short column purification (5-10% MeOH in DCM) provided the product (640 mg, 2.284 mmol, 87%) as a yellow powder. MS (ESI) 281.1 [M+1]⁺.

### Step 6: 1-bromo-8-(difluoromethoxy)-6-methoxy-3-methylbenzo[e]imidazo[5,1-c][1,2,4]triazine

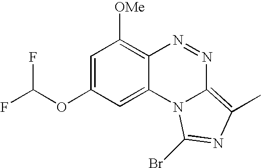

To a mixture of 8-(difluoromethoxy)-6-methoxy-3-methylbenzo[e]imidazo[5,1-c][1,2,4]triazine (592 mg, 2.113 mmol) and N-bromosuccinimide (564 mg, 3.17 mmol) in a flask was added acetonitrile (20 ml) under nitrogen at RT. The resulting mixture was stirred at RT in darkness overnight. Quenched with the reaction with sodium sulfite solution, extracted with ethyl acetate. Column purification (30-50% EtOAc in DCM) provided the product (700 mg, 1.949 mmol, 92%) as a yellow powder. MS (ESI) 359.0 [M+1]⁺.

### Step 7: General Suzuki Coupling: Preparation of compounds in Table 7

A mixture of 1-bromo-8-(difluoromethoxy)-6-methoxy-3-methylbenzo[e]imidazo[5,1-c][1,2,4]triazine (60 mg, 0.167 mmol), various boronic acids (2 equivalents) or boronic ester (5 equivalents), potassium carbonate (69.3 mg, 0.501 mmol) and Pd(PPh₃)₄ (9.65 mg, 8.35 μmol) in a 40 mL vial was vacuumed and refilled with nitrogen, followed by addition of dioxane (4.5 ml) and water (1.500 ml). The resulting mixture was stirred at 90° C. overnight. Cooled to RT. Solvent was removed by rotavap. The residue was purified by column to provide the product.

The following compounds (Table 7) were prepared following the procedure described above.

TABLE 7

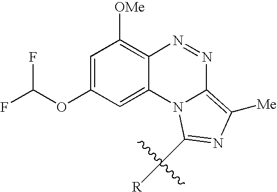

| Example | | | MS |
|---|---|---|---|
| # | R | Chemical Name and NMR | [M + 1]⁺ |
| 160 | 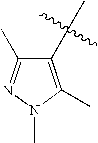 | 8-(difluoromethoxy)-6-methoxy-3-methyl-1- (1,3,5-trimethyl-1H-pyrazol-4- yl)benzo[e]imidazo[5,1-c][1,2,4]triazine ¹H NMR (400 MHz, d₆-DMSO) δ ppm 7.22 (t, J = 7.28 Hz, 1H), 7.05 (d, J = 2.3 Hz, 1H), 6.72 (d, J = 2.3 Hz, 1H), 4.05 (s, 3H), 3.76 (s, 3H), 2.75 (s, 3H), 2.07 (s, 3H), 1.93 (s, 3H). | 389.2 |
| 161 | 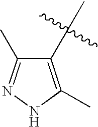 | 8-(difluoromethoxy)-1-(3,5-dimethyl-1H- pyrazol-4-yl)-6-methoxy-3- methylbenzo[e]imidazo[5,1-c][1,2,4]triazine ¹H NMR (400 MHz, d₆-DMSO) δ ppm 12.79 (s, 1H), 7.22 (t, J = 72.8 Hz, 1H), 7.05 (d, J = 2.3 Hz, 1H), 6.70 (d, J = 2.3 Hz, 1H), 4.05 (s, 3H), 3.76 (s, 3H), 2.75 (s, 3H), 2.07 (s, 3H), 1.95 (s, 3H). | 375.1 |
| 162 | 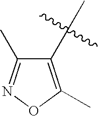 | 4-(8-difluoromethoxy)-6-methoxy-3- methylbenzo[e]imidazo[5,1-c][1,2,4]triazin- 1-yl)-3,5-dimethylisoxazole ¹H NMR (400 MHz, d₆-DMSO) δ ppm 7.31 (t, J = 72.8 Hz, 1H), 7.11 (d, J = 2.3 Hz, 1H), 6.67 (d, J = 2.3 Hz, 1H), 4.07 (s, 3H), 2.77 (s, 3H), 2.32 (s, 3H), 2.08 (s, 3H). | 376.1 |
| 163 | 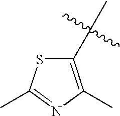 | 5-(8-difluoromethoxy)-6-methoxy-3- methylbenzo[e]imidazo[5,1-c][1,2,4]triazin- 1-yl)-2,4-dimethylthiazole ¹H NMR (400 MHz, d₆-DMSO) δ ppm 7.22 (t, J = 72.7 Hz, 1H), 7.09 (d, J = 2.3 Hz, 1H), 6.55 (d, J = 2.3 Hz, 1H), 4.06 (s, 3H), 2.75 (s, 3H), 2.72 (s, 3H), 2.09 (s, 3H). | 392.1 |
| 164 | 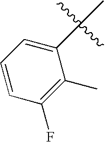 | 8-(difluoromethoxy)-1-(3-fluoro-2- methylphenyl)-6-methoxy-3- methylbenzo[e]imidazo[5,1-c][1,2,4]triazine ¹H NMR (400 MHz, d₆-DMSO) δ ppm 7.40- 7.50 (m, 2H), 7.35 (m, 1H), 7.08 (t, J = 72.6 Hz, 1H), 7.03 (d, J = 2.3 Hz, 1H), 6.15 (d, J = 2.3 Hz, 1H), 4.04 (s, 3H), 2.78 (s, 3H), 1.92 (s, 3H). | 389.1 |
| 165 | 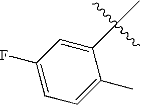 | 8-(difluoromethoxy)-1-(5-fluoro-2- methylphenyl)-6-methoxy-3- methylbenzo[e]imidazo[5,1-c][1,2,4]triazine ¹H NMR (400 MHz, d₆-DMSO) δ ppm 7.50 (m, 1H), 7.38-7.46 (m, 2H), 7.09 (t, J = 72.8 Hz, 1H), 7.05 (d, J = 2.3 Hz, 1H), 6.16 (d, J = 2.3 Hz, 1H), 4.05 (s, 3H), 2.78 (s, 3H), 1.97 (s, 3H). | 389.1 |
| 166 | 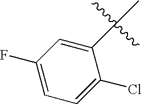 | 1-(2-chloro-5-fluorophenyl)-8- (difluoromethoxy)-6-methoxy-3- methylbenzo[e]imidazo[5,1-c][1,2,4]triazine ¹H NMR (400 MHz, d₆-DMSO) δ ppm 7.78 (m, 1H), 7.70 (m, 1H), 7.62 (m, 1H), 7.15 (t, J = 72.6 Hz, 1H), 7.08 (d, J = 2.3 Hz, 1H), 6.19 (d, J = 2.3 Hz, 1H), 4.06 (s, 3H), 2.79 (s, 3H). | 409.1 |
| 167 | 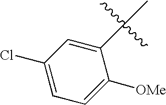 | 1-(5-chloro-2-methoxyphenyl)-8- (difluoromethoxy)-6-methoxy-3- methylbenzo[e]imidazo[5,1-c][1,2,4]triazine ¹H NMR (400 MHz, d₆-DMSO) δ ppm 7.71 (dd, J = 9.0, 2.6 Hz, 1H), 7.62 (d, J = 2.6 Hz, 1H), 7.26 (d, J = 9.0 Hz, 1H), 7.15 (t, J = 72.8 Hz, 1H), 7.07 (d, J = 2.3 Hz, 1H), 6.43 (d, J = 2.3 Hz, 1H), 4.06 (s, 3H), 2.60 (s, 3H), 2.77 (s, 3H). | 421.1 |
| 168 | 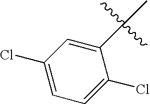 | 1-(2,5-dichlorophenyl)-8-(difluoromethoxy)- 6-methoxy-3-methylimidazo[5,1- c][1,2,4]benzotriazine ¹H NMR (400 MHz, d₆-DMSO) δ ppm 7.81 (s, 1H), 7.81 (m, 2H), 7.17 (t, J = 72.6, 1H), 7.10 (s, 1H), 6.22 (s, 1H), 4.07 (s, 3H), 2.79 (s, 3H). | 426.1 |
| 169 | 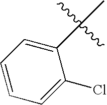 | 1-(2-chlorophenyl)-8-(difluoromethoxy)-6- methoxy-3-methylimidazo[5,1- c][1,2,4]benzotriazine ¹H NMR (400 MHz, d₆-DMSO) δ ppm 7.73 (m, 3H), 7.61 (m, 1H), 7.09 (t, J = 72.8, 1H), 7.06 (s, 1H), 6.14 (s, 1H), 4.05 (s, 3H), 2.78 (s, 3H). | 391.1 |

### Example 170

### 8-(difluoromethoxy)-6-methoxy-1-(5-methoxypyridin-3-yl)-3-methylimidazo[5,1-c][1,2,4]benzotriazine

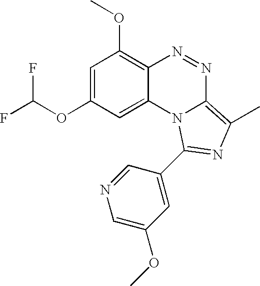

### Step 1. 1-(5-fluoro-3-methoxy-2-nitrophenyl)-4-methyl-1H-imidazole

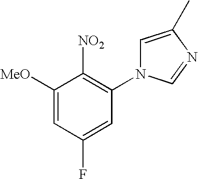

1,5-difluoro-3-methoxy-2-nitrobenzene (7 g, 37.0 mmol) and 4-methyl-1H-imidazole (3.04 g, 37.0 mmol) were dissloved in DMF (100 mL). To this was added potassium carbonate (10 g, 74 mmol)). The reaction was stirred at RT overnight then poured into water and extracted with ethyl acetate. The organic layer was separated, washed with water, brine, then dried over MgSO₄ and filtered. The solvent was removed under reduced pressure and the crude purified by flash chromatography on silica gel in 50% to 70%. Ethyl acetate/hexane 4.5 g of a pale yellow solid was recovered. MS [(+)ESI] m/z=252.1 [M−H]⁺.

### Step 2. 2-bromo-1-(5-fluoro-3-methoxy-2-nitrophenyl)-4-methyl-1H-imidazole

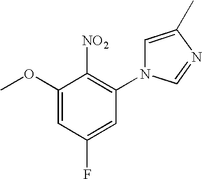

1-(5-fluoro-3-methoxy-2-nitrophenyl)-4-methyl-1H-imidazole (4.9 g, 19.51 mmol) was dissolved in THF (40 mL) and cooled to 0° C. To this was added 1-bromopyrrolidine-2,5-dione (3.65 g, 20.48 mmol). The reaction was let warm to RT after 1 h and stirred for 4 h. The reaction was poured into water and extracted with ethyl acetate. The organic layer was separated and combined then brine, dried over MgSO₄ and filtered. The solvent was removed under reduced pressure and the crude purified by flash chromatography on silica gel in 20% to 40% ethyl acetate/hexane. 1.6 g of a waxy yellow solid was collected. MS [(+)ESI] m/z=330.0 [M−H]⁺.

### Step 3. 3-(2-bromo-4-methyl-1H-imidazol-1-yl)-5-methoxy-4-nitrophenol

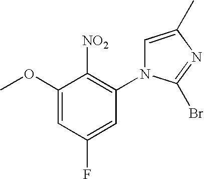

To 2-bromo-1-(5-fluoro-3-methoxy-2-nitrophenyl)-4-methyl-1H-imidazole (1.6 g, 4.85 mmol) was added 2-(methylsulfonyl)ethanol (0.903 g, 7.27 mmol) dissolved in DMF (10 mL), followed by powdered potassium hydroxide (0.81 g, 14.5 mmol). The reaction was stirred for 1 h at RT then poured into saturated ammonium chloride and extracted with ethyl acetate. The organic layer was separated and washed with water then brine, dried over MgSO₄ and the solvent removed under reduced pressure, 1.6 g of a yellow solid was collected. MS [(+)ESI] m/z=328.0 [M−H]⁺.

### Step 4. 2-bromo-1-(5-(difluoromethoxy)-3-methoxy-2-nitrophenyl)-4-methyl-1H-imidazole

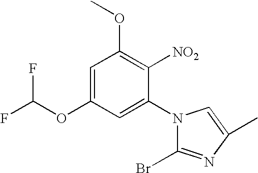

3-(2-bromo-4-methyl-1H-imidazol-1-yl)-5-methoxy-4-nitrophenol (0.5 g, 1.524 mmol) and potassium carbonate (0.25 g, 0.18 mmol) were suspended in a solution containing DMF (6 mL) and water (0.6 mL). The reaction was heated to 80° C. and sodium 2-chloro-2,2-difluoroacetate (0.465 g, 3.05 mmol) was added in one portion. The reaction was brought to 110° C. and maintained for 1 h, then poured into saturated ammonium chloride and extracted with ethyl acetate. The organic layer was separated and washed with water then brine and dried over MgSO₄ and filtered. The solvent was removed under reduced pressure and the crude purified by flash chromatography on silica gel in 20 to 50% ethyl acetate/hexane. 0.42 g of a tan solid was recovered. MS [(+)ESI] m/z=378.0 [M−H]⁺.

### Step 5. 3-(1-(5-(difluoromethoxy)-3-methoxy-2-nitrophenyl)-4-methyl-1H-imidazol-2-yl)-5-methoxypyridine

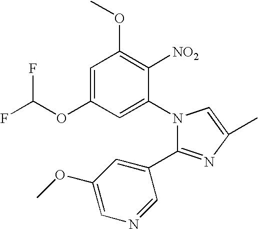

2-bromo-1-(5-(difluoromethoxy)-3-methoxy-2-nitrophenyl)-4-methyl-1H-imidazole (0.15 g, 0.397 mmol) and 5-methoxypyridin-3-ylboronic acid (0.121 g, 0.793 mmol) were suspended in a solution containing dioxane (4 mL) and water (1 mL). To this was added potassium carbonate (162 mg, 1.2 mmol). Argon was bubbled through the reaction for 1 min and Pd(PPh₃)₄ (10% mol, 46 mg) was added. The reaction was sealed and heated to 110° C. for 16 h. The reaction was poured into water and extracted with ethyl acetate. The organic layer was separated and dried over MgSO₄, filtered, and the solvent removed under reduced pressure. The crude was purified by flash chromatography on silica gel in 50-100% ethyl acetate/hexane. 0.15 g of a clear oil was recovered. MS [(+)ESI] m/z=407.1 [M−H]⁺.

### Step 6. 4-(difluoromethoxy)-2-methoxy-6-(2-(5-methoxypyridin-3-yl)-4-methyl-1H-imidazol-1-yl)aniline

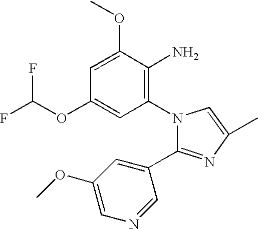

3-(1-(5-(difluoromethoxy)-3-methoxy-2-nitrophenyl)-4-methyl-1H-imidazol-2-yl)-5-methoxypyridine (0.15 g, 0.369 mmol) was dissolved in a solution containing ethanol (2 mL) and glacial acetic acid (2 mL). To this was added Fe powder (0.0.124 g, 2.2 mmol). The reaction was heated to 100° C. for 1 h, the reaction was poured into 1N NaOH and extracted with ethyl acetate. The organic layer was separated and washed with water, brine and dried over MgSO₄. The solution was filtered and the solvent removed under reduced pressure. 130 mg of an off white solid was recovered. MS [(+)ESI] m/z=377.1 [M−H]⁺.

### Step 7: 8-(difluoromethoxy)-6-methoxy-1-(5-methoxypyridin-3-yl)-3-methylimidazo[5,1-c][1,2,4]benzotriazine

4-(difluoromethoxy)-2-methoxy-6-(2-(5-methoxypyridin-3-yl)-4-methyl-1H-imidazol-1-yl)aniline (0.12 g, 0.319 mmol) was dissolved in glacial acetic acid (4 mL). To this was added sodium nitrite (24 mg, 0.33 mmol). The reaction was stirred at RT for 1 h, then poured into 1N NaOH and extracted with ethyl acetate. The organic layer was separated, washed with water then brine, dried over MgSO₄ and filtered. The solvent was removed under reduced pressure and the crude recrystallized from ethyl acetate/hexane. 50 mg of a yellow solid was recovered. [(+)ESI] m/z=388.1 [M+H]⁺. ¹H NMR (400 MHz, d₆-DMSO) δ ppm 8.51 (s, 1H), 8.47 (s. 1H), 7.76 (s, 1H), 7.19 (t, J=72.7, 1H), 7.07 (m, 1H), 6.52 (s, 1H), 4.08 (s, 3H), 3.86 (s, 3H), 2.77 (s, 3H).

### Example 171

### 1,8-Bis-(2,5-dichloro-phenyl)-3-methyl-imidazo[5,1-c][1,2,4]benzotriazine

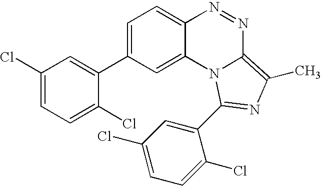

### Step 1. 1-bromo-8-chloro-3-methylbenzo[e]imidazo[5,1-c][1,2,4]triazine

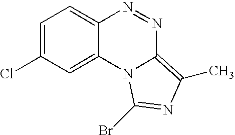

This compound was prepared as described in Example 132, step 6 by replacing 6-chloro-3-methyl-8-(trifluoromethyl)benzo[e]imidazo[5,1-c][1,2,4]triazine with 8-chloro-3-methylbenzo[e]imidazo[5,1-c][1,2,4]triazine (Example 42).

### Step 2. 1,8-Bis-(2,5-dichloro-phenyl)-3-methyl-imidazo[5,1-c][1,2,4]benzotriazine

211 mg of 1-bromo-8-chloro-3-methylbenzo[e]imidazo[5,1-c][1,2,4]triazine was dissolved in 25 ml dioxane. An second solution of 1 g K₂CO₃ in 3.2 ml water is added. To this mixture 350 mg 2,5-dichlorophenyl boronic acid and 200 mg Pd(PPh₃)₄ are added. The resulting mixture is heated up to reflux for 3 h. At RT the solution is filtered off. The solvent is distilled off from the clear solution. The remaining residue is stirred with 50 ml water for 30 minutes. The product suspension stays overnight. The crude product is collected by filtration and purified by flash chromatography. Yield: 105 mg; MS [M+H]⁺ 473; m.p.: 190-193° C.

The following compounds of Table 8 were prepared analogous to the procedure for Example 171.

TABLE 8

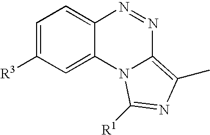

| Example | | | MS | m.p |
|---|---|---|---|---|
| # | R¹, R³ | Chemical Name | [M + 1]⁺ | (° C.) |
| 172 | 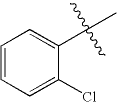 | 1,8-Bis-(2-chloro-phenyl)-3-methyl- imidazo[5,1-c][1,2,4]benzotriazine | 405 | 172-175 |
| 173 | 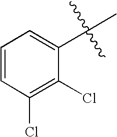 | 1,8-Bis-(2,3-dichloro-phenyl)-3- methyl-imidazo[5,1- c][1,2,4]benzotriazine | 473 | 207-210 |
| 174 | 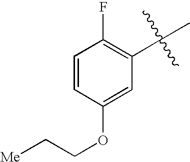 | 1,8-Bis-(2-fluoro-5-propoxy-phenyl)- 3-methyl-imidazo[5,1- c][1,2,4]benzotriazine | 489 | 134-136 |
| 175 | 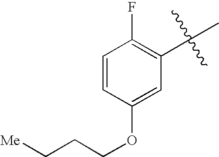 | 1,8-Bis-(5-butoxy-2-fluoro-phenyl)-3- methyl-imidazo[5,1- c][1,2,4]benzotriazine | 517 | 94-95 |
| 176 | 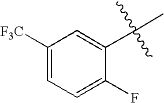 | 1,8-Bis-(2-fluoro-5-trifluoromethyl- phenyl)-3-methyl-imidazo[5,1- c][1,2,4]benzotriazine | 509 | 155-158 |
| 177 | 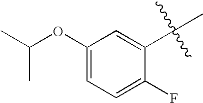 | 1,8-Bis-(2-fluoro-5-isopropoxy- phenyl)-3-methyl-imidazo[5,1- c][1,2,4]benzotriazine | 489 | 160.5-162 |
| 178 | 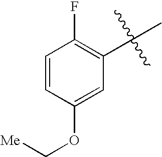 | 1,8-Bis-(5-ethoxy-2-fluoro-phenyl)-3- methyl-imidazo[5,1- c][1,2,4]benzotriazine | 461 | 152-155 |

### Examples 179

### 1-Cyclohexyl-8-(2-cyclohexyl-4-methyl-imidazol-1-yl)-3-methyl-imidazo[5,1-c][1,2,4]benzotriazine

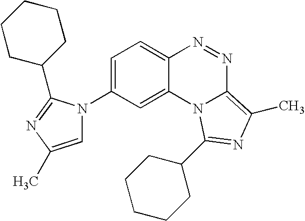

### Step 1: 4-fluoro-2-(2-cyclohexyl-4-methyl-imidazol-1-yl)-nitrobenzene

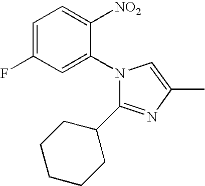

To a suspension of K₂CO₃ (6 g), 2-cyclohexyl-4-methyl imidazole (3 g) and 50 ml acetonitrile was added 2,4-difluoro-nitrobenzene (3.2 g). The reaction mixture was stirred and heated to reflux for 5 h. Then the reaction mixture was filtered off. The solvent was removed and the crude residue was purified by chromatography (DCM/methanol 96:4). Yield: 2.0 g

### Step 2: 2,4-bis-(2-cyclohexyl-4-methyl-imidazol-1-yl)-nitrobenzene

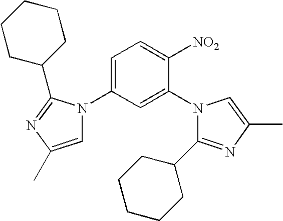

To a suspension of CsCO₃ (6 g), 2-cyclohexyl-4-methyl imidazole (3 g) and 50 ml acetonitrile was added 4-fluoro-2-(2-cyclohexyl-4-methyl-imidazol-1-yl)-nitrobenzene (2.0 g). The reaction mixture was stirred and heated to reflux for 7 h. Then the reaction mixture was filtered off. The solvent was removed and the crude residue was purified by chromatography (DCM/methanol 96:4). Yield: 1.25 g

### Step 3: 1-amino-2,4-bis-(2-cyclohexyl-4-methyl-imidazol-1-yl)-benzene

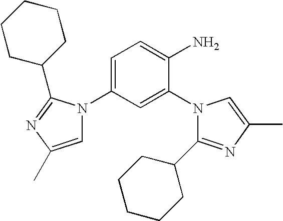

A mixture of 2,4-bis-(2-cyclohexyl-4-methyl-imidazol-1-yl)-nitrobenzene (1.25 g), 15 ml methanol, 3 ml water, 1.5 ml hydrazine hydrate and Raney-Ni catalyst (1 g) was stirred at room temperature for 2 hours. During this time an additional portion of 0.5 ml hydrazine hydrate was added after 30 minutes and a second portion of 0.5 ml hydrazine hydrate after 1 hour. The catalyst was filtered off 30 ml water and 100 ml ethyl acetate were added to the reaction mixture which was stirred again for 30 minutes. The organic layer was separated and the solvent was removed. The residual crude product is used without further purification. Yield: 1.0 g

### Step 4: 1-Cyclohexyl-8-(2-cyclohexyl-4-methyl-imidazol-1-yl)-3-methyl-imidazo[5,1-c]-[1,2,4]benzotriazine

1-amino-2,4-bis-(2-cyclohexyl-4-methyl-imidazol-1-yl)-benzene (1.0 g) was stirred with 15 ml 1M H₂SO₄ at 0° C. A solution of sodium nitrite (0.8 g) in 10 ml water was added to the solution over a period of 30 minutes. The mixture was stirred for additional 2 hours at about 0° C. 10 ml water were added. The mixture is neutralized with solid NaHCO₃ stepwise to pH=9. The crude product precipitated. It was separated and washed with 20 ml water. The product was purified by chromatography (DCM/methanol 95:5). Yield: 0.25 g, m.p.: 224-227° C.; MS [M+H]⁺=429.

### Examples 180

### 1-(2,5-Dichloro-phenyl)-3-methyl-8-piperidin-1-yl-imidazo[5,1-c][1,2,4]benzotriazine

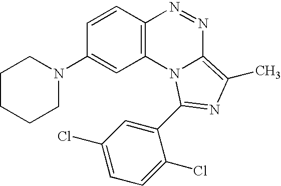

This compound was prepared according to the procedure described for Example 179 by replacing 2-cyclohexyl-4-methyl imidazole with 2-(2,5-dichlorophenyl)-4-methyl imidazole (from Example 5, step 1) in step 1 and by replacing 2-cyclohexyl-4-methyl imidazole with piperidine in step 2. m.p.: 158-160° C.; MS [M+H]⁺=412.

### Examples 181

### 1-(2,5-Dichloro-phenyl)-3-methyl-8-morpholin-4-yl-imidazo[5,1-c][1,2,4]benzotriazine

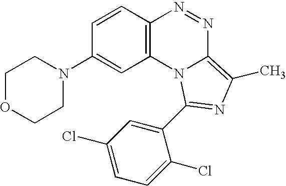

This compound was prepared according to the procedure described for Example 179 by replacing 2-cyclohexyl-4-methyl imidazole with 2-(2,5-dichlorophenyl)-4-methyl imidazole (from Example 5, step 1) in step 1 and by replacing 2-cyclohexyl-4-methyl imidazole with morpholine in step 2. m.p.: 239-242° C.; MS [M+H]⁺=414.

### Examples 182

### 1-Isobutyl-8-(2-isobutyl-4-methyl-imidazol-1-yl)-3-methyl-imidazo[5,1-c][1,2,4]benzotriazine

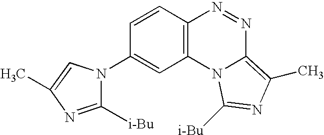

This compound was prepared according to the procedure described for Example 179 by replacing 2-cyclohexyl-4-methyl imidazole with 2-(iso-butyl)-4-methyl imidazole in steps 1 and 2. m.p.: 134-136° C.; MS [M+H]⁺=377.

### Examples 183

### 1-(2-Chloro-phenyl)-3-methyl-8-piperidin-1-yl-imidazo[5,1-c][1,2,4]benzotriazine

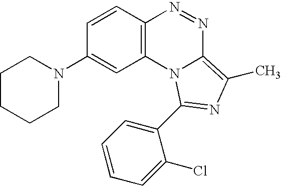

This compound was prepared according to the procedure described for Example 179 by replacing 2-cyclohexyl-4-methyl imidazole with 2-(2-chlorophenyl)-4-methyl imidazole in step 1 and by replacing 2-cyclohexyl-4-methyl imidazole with piperidine in step 2. m.p.: 207-210° C.; MS [M+H]⁺=378.

### Examples 184-187

The following compounds of Table 9 were prepared analogous to the procedure for Example 183.

TABLE 9

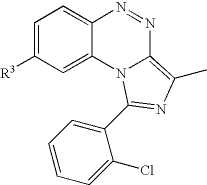

| Example | | | MS | m.p |
|---|---|---|---|---|
| # | R³ | Chemical Name | [M + 1]⁺ | (° C.) |
| 184 | 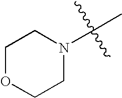 | 1-(2-Chloro-phenyl)-3-methyl-8-morpholin- 4-yl-imidazo[5,1-c][1,2,4]benzotriazine | 380 | 242-245 |
| 185 | 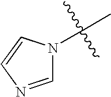 | 1-(2-Chloro-phenyl)-8-imidazol-1-yl-3- methyl-imidazo[5,1-c][1,2,4]benzotriazine | 361 | 95-98 |
| 186 | 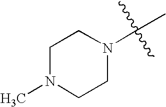 | 1-(2-Chloro-phenyl)-3-methyl-8-(4-methyl- piperazin-1-yl)-imidazo[5,1- c][1,2,4]benzotriazine | 393 | 230 |
| 187 | 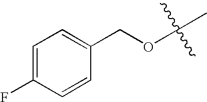 | 1-(2-Chloro-phenyl)-8-(4-fluoro- benzyloxy)-3-methyl-imidazo[5,1- c][1,2,4]benzotriazine | 419 | 174-177 |

Certain compounds of formula (I) are inhibitors of the enzyme PDE2. A substance is considered to effectively inhibit PDE2 if it has an IC₅₀ of less than 10 μM, preferably less than 1 μM. Certain compounds of formula (I) are inhibitors of the enzyme PDE10. A substance is considered to effectively inhibit PDE10 if it has an IC₅₀ of less than 10 μM, preferably less than 1 μM.

### Example A

### Inhibition of Recombinant PDE2A (Expressed in Baculovirus/SF21-Cells)

PDE2A (NM002599) was cloned and the gene was inserted in the baculovirus and the enzyme-protein expressed in SF21-cells. The enzyme was isolated from these cells by harvesting the cells by an centrifugation at 200 g to collect the cells. The cells were resuspended in 50 mM Tris-HCl/5 mM MgCl2 buffer (pH=7.4) (Sigma, Deisenhofen, Germany; Merck, Darmstadt, Germany) and lysed by a sonication of the cells (three times for 15 seconds, Labsonic U, Fa. Braun, Degersheim, Switzerland, level “high”). The membrane fraction of PDE2A was obtained by a centrifugation at 48 000 g for 1 h, resuspended in buffer and stored at −70° C.

PDE2A activity was determined in a one step procedure in microtiterplates. The reaction mixture of 100 μl contained 50 mM Tris-HCl/5 mM MgCl2 buffer (pH=7.4) (Sigma, Deisenhofen, Germany; Merck, Darmstadt, Germany), 0.5 μM [3H]-cAMP (Amersham, Buckinghamshire, UK), 1000 nM cGMP and the enzyme. Non-specific enzyme activity was tested in the absence of cGMP. The reaction was initiated by addition of the substrate solution and was carried out at 37° C. for 30 minutes. Enzymatic activity then was stopped by addition of 25 μl SPA-beads (Amersham-Pharmacia). One hour later the mixture was measured in a liquid scintillation counter for microtiterplates (Microbeta Trilux). For pipetting of the incubation mixture the robot Biomek (Fa. Beckman) was routinely used.

The determined Km for this assay was Kₘ=4200 nmol/l for the membrane fraction and Kₘ=5300 nM for the cytosolic fraction. The optimal amount of enzyme in the assay has been determined and optimised for each enzyme preparation separately before using the enzyme in compound testing. For determination of IC₅₀ values the Hill-plot, 2-parameter-model, was used.

### Example B

### Inhibition of Recombinant PDE10A (Baculovirus/SF21 System)

The DNA of PDE10A1 (AB 020593, 2340 bp) was synthesized and cloned into the vector pCR4.TOPO (Entelechon GmbH, Regensburg, Germany). The gene was than inserted into a baculovirus vector, ligated with the baculovirus DNA. The enzyme-protein was expressed in SF21-cells. The enzyme was isolated from these cells by harvesting the cells by an centrifugation at 500 g to collect the cells.

The cells were resuspended in 50 mM Tris-HCl/1 mM EDTA/250 mM Sucrose buffer, pH=7.4 (Sigma, Deisenhofen, Germany; Merck, Darmstadt, Germany) and lysed by sonification of the cells (three times for 15 seconds, Labsonic U, Fa. Braun, Degersheim, Switzerland, level “high”). The cytosolic PDE10A was obtained by a centrifugation at 48,000 g for 1 h in the supernatant and stored at −70° C.

PDE activity was determined in a one step procedure in microtiter plates. The reaction mixture of 100 μl contained 50 mM Tris-HCl/5 mM MgCl2 buffer (pH=7.4, Sigma, Deisenhofen, Germany; Merck, Darmstadt, Germany) 0.1 μM [3H]-cAMP (Amersham, Buckinghamshire, UK) and the enzyme. Non-specific enzyme activity was determined without the enzyme. The reaction was initiated by addition of the substrate solution and was carried out at 37° C. for 30 minutes. Enzymatic activity then was stopped by addition of 25 μl Ysi-SPA-beads (Amersham-Pharmacia). One hour later the mixture was measured in a liquid scintillation counter for microtiter plates (Microbeta Trilux). The Biomek 2000 (Beckman) was used routinely for pipetting of the incubation mixture. The optimal amount of enzyme in the assay has been determined and optimized for each enzyme preparation separately before using the enzyme in compound testing. For determination of IC₅₀ values the Hill-plot, 2-parameter-model, was used.

Table of IC₅₀ data for PDE2A and PDE10A assays*

| Example | PDE10A | PDE2A |
|---|---|---|
| # | IC₅₀ (nM) | IC₅₀ (nM) |
| 1 | 626 | 716 |
| 2 | 401 | 546 |
| 3 | 3898 | 3760 |
| 4 | 228 | 541 |
| 5 | 23.7 | 3.31 |
| 6 | 761 | 29.8 |
| 7 | 258 | 17.8 |
| 8 | 176 | 36.5 |
| 9 | 156 | 302 |
| 10 | 410 | 1530 |
| 11 | >10000 | >10000 |
| 12 | 780 | 187 |
| 13 | 86 | 32.6 |
| 14 | 308 | 371 |
| 15 | >5000 | >1000 |
| 16 | 456 | 351 |
| 17 | 196 | 122 |
| 18 | 183 | 20.1 |
| 19 | 2030 | 1360 |
| 20 | 2290 | 473 |
| 21 | >1000 | 912 |
| 22 | >500 | 37.7 |
| 23 | 1540 | 59.1 |
| 24 | >500 | 240 |
| 25 | 1490 | 341 |
| 26 | 635 | 500 |
| 27 | 911 | 298 |
| 28 | 359 | 122 |
| 29 | 425 | 35.3 |
| 30 | >1000 | 1790 |
| 31 | 5340 | 551 |
| 32 | — | — |
| 33 | — | — |
| 34 | 75.2 | 26.7 |
| 35 | >5000 | >5000 |
| 36 | 351 | 73.4 |
| 37 | 77.2 | 5.31 |
| 38 | 207 | 41.45 |
| 39 | 115 | 89.6 |
| 40 | 3940 | 1410 |
| 41 | 3120 | >1000 |
| 42 | >10000 | >10000 |
| 43 | >500 | 233 |
| 44 | 1550 | 121 |
| 45 | 4.16 | 775 |
| 46 | 0.28 | 30.3 |
| 47 | 2.06 | 589 |
| 48 | 1.48 | 196 |
| 49 | 5.13 | 95.9 |
| 50 | 71.2 | 2190 |
| 51 | 0.7 | 210 |
| 52 | 1.85 | 292 |
| 53 | 6.12 | 4200 |
| 54 | 256 | 3370 |
| 55 | 0.45 | 56.9 |
| 56 | 0.73 | 47.1 |
| 57 | 3.35 | 522 |
| 58 | 40.9 | 35.5 |
| 59 | 260 | 3010 |
| 60 | 3.31 | 172 |
| 61 | 2.41 | 103 |
| 62 | 8.34 | 442 |
| 63 | 3.99 | 437 |
| 64 | 1.92 | 70.4 |
| 65 | 0.85 | 50.4 |
| 66 | 1.53 | 110 |
| 67 | 16.8 | 64.85 |
| 68 | 64.2 | 109 |
| 69 | 34.7 | 55.5 |
| 70 | 12.5 | 22.8 |
| 71 | 11.4 | 5.53 |
| 72 | 7.77 | 14.4 |
| 73 | 18.9 | 15.1 |
| 74 | 38.5 | 134 |
| 75 | 15 | 13.9 |
| 76 | 9.6 | 9.3 |
| 77 | 30.9 | 10.3 |
| 78 | 8.51 | 3.32 |
| 79 | 15.2 | 8.39 |
| 80 | 7.09 | 9.43 |
| 81 | 9.43 | 35.9 |
| 82 | 199 | 60.7 |
| 83 | 65.7 | 16.5 |
| 84 | 10.7 | 60.95 |
| 85 | 55.2 | 37.3 |
| 86 | 8.33 | 3390 |
| 87 | 246 | 1250 |
| 88 | 7.99 | 14.2 |
| 89 | 623 | 819 |
| 90 | 386 | 585 |
| 91 | 43.6 | 56.1 |
| 92 | 523 | 469 |
| 93 | 252 | 312 |
| 94 | 122 | 127 |
| 95 | 45.5 | 39.4 |
| 96 | 53.1 | 40.3 |
| 97 | 146 | 146 |
| 98 | 15.4 | 45 |
| 99 | 9.11 | 19998 |
| 100 | 6.53 | 894 |
| 101 | 43 | 258 |
| 102 | 14.5 | 704 |
| 103 | 4.72 | 435 |
| 104 | 5.37 | 171 |
| 105 | 7.13 | — |
| 106 | 7.91 | 336 |
| 107 | 2.98 | 62.5 |
| 108 | 8.37 | 55 |
| 109 | 102 | >5000 |
| 110 | 8.52 | 609 |
| 111 | 3.01 | 324 |
| 112 | 1.88 | 636 |
| 113 | 5.09 | 591 |
| 114 | 17.1 | 1060 |
| 115 | 67 | >10000 |
| 116 | 13.1 | 1500 |
| 117 | 3.55 | 1150 |
| 118 | 2.02 | — |
| 119 | 1.64 | 269 |
| 120 | 53.6 | >10000 |
| 121 | 133 | >1000 |
| 122 | 39.3 | 1620 |
| 123 | 25.4 | 2375.5 |
| 124 | 13 | 1430 |
| 125 | 12.1 | 117 |
| 126 | 2.45 | 108 |
| 127 | 11.7 | 3420 |
| 128 | 6.31 | 654 |
| 129 | 61.6 | 4950 |
| 130 | 7.84 | 1270 |
| 131 | 7.20 | 1220 |
| 132 | 32.7 | — |
| 133 | 9.69 | 403 |
| 134 | 1.48 | 346.5 |
| 135 | — | — |
| 136 | — | — |
| 137 | — | — |
| 138 | — | — |
| 139 | — | — |
| 140 | — | — |
| 141 | — | — |
| 142 | 10.6 | 1220 |
| 143 | 57 | >1000 |
| 144 | — | — |
| 145 | — | — |
| 146 | — | — |
| 149 | 22 | 451 |
| 150 | 7 | 944 |
| 151 | 0.7 | 80.6 |
| 152 | 3.05 | 105 |
| 153 | — | — |
| 154 | 0.67 | 73.9 |
| 155 | — | — |
| 156 | — | — |
| 157 | 5.32 | 277 |
| 158 | — | — |
| 159 | — | — |
| 160 | — | — |
| 161 | — | — |
| 162 | — | — |
| 163 | — | — |
| 164 | — | — |
| 165 | — | — |
| 166 | — | — |
| 167 | — | — |
| 168 | — | — |
| 169 | — | — |
| 170 | — | — |
| 171 | — | — |
| 172 | 6.55 | 1.28 |
| 173 | 57.6 | 61.5 |
| 174 | 41.1 | 3.55 |
| 175 | 21.6 | 0.77 |
| 176 | 117 | 137 |
| 177 | — | — |
| 178 | — | — |
| 179 | 322 | 242 |
| 180 | 49.55 | 6.9 |
| 181 | 24.3 | 2.3 |
| 182 | 32.8 | 134 |
| 183 | 12.1 | 7.79 |
| 184 | 9.77 | 6.39 |
| 185 | 8.97 | 7.14 |
| 186 | 9.02 | 7.49 |
| 187 | 24.6 | 9.72 |

The compounds of formula (I) show significant antidepressant, anxiolytic and cognition enhancing effects in vivo.

### Example C

### Novel Object Recognition

The novel object recognition is an animal model of learning and memory (Rutten et al., 2006a+b).

The novel object recognition is performed in glass aquaria (40×60×40 cm) that have 3 black walls and one transparent wall. The floor consists of black, antislip PVC. Objects of different material (iron, plastic, coated hardwood) and forms and similar size are used for the experiment. The objects are positioned 10 cm from the wall and 35-40 cm from each other. Female Wistar-rats are used for this experiment.

One day before the experiment rats have 15 min to habituate to the arena and two objects.

On the first day of the experiment rats are placed into the arena and have five min to explore two equal objects. To disturb the learning process, MK-801 at 0.025 mg/kg is administered intraperitoneally on the first day of the experiment 30 min before the test starts.

On the second day of the experiment (24 h later) rats are again placed into the arena and have 5 min to explore one of the old objects and a novel object. The position of the novel object is changed from rat to rat to avoid a place preference.

The following parameters are recorded:

1. the time the rats spent with each object on the first day
2. the time the rats spent with each object on the second day
3. percent of time rats spent with the novel object on the second day

Exploratory contact is regarded as the nose of the rat being within a 2-cm-radius of an object.

Vehicle or compounds of formula (I) are given orally as a suspension on the first day of experiment 30 min prior to the test session.

### Example D

### Forced Swim Test

The forced swim test is an established animal model of depression (Yacoubi et al., 2001). Mice which are forced to swim in a restricted area from which they cannot escape will rapidly cease attempts to escape and adopt a characteristic immobile posture which can be readily identified and timed. Immobility is taken as depression-related behaviour in the animal (Porsolt, 1979).

For the test a glass cylinder (height: 20 cm, internal diameter: 15 cm) containing 11 cm water maintained at 23° C. is used. On the day of experiment the mice are forced to swim in the water for 6 min and the immobility time is recorded during the last 4 min of the 6-min-period. Afterwards animals are removed from the water, dried with a paper towel and put under infrared light.

### Example E

### Light and Dark Box

The light and dark box is an established animal model of anxiety (Crawley, 1985). The light and dark box consists of two chambers (each 30×30 cm) that are connected by an opening. There is an aversive chamber with white walls that is brightly lit (600 lux) and a dark chamber with black walls that is only lit by an infrared lamp (150 lux).

Untreated mice predominately stay in the dark chamber whereas mice treated with an anxiolytic compound go more often into the light chamber resulting in an increased number of transitions between the boxes and increased time in the light box. In addition the distance traveled in the dark chamber is regarded as an activity-related parameter.

For the experiment, mice are placed in the light box after the pre-treatment time. Recording time starts when the mouse enters the dark box for the first time. Then the animal has 5 min to explore the two chambers.

The behaviour of the mice is recorded by video and analyzed by VideoMot 2 (TSE systems, Germany). The following parameters are recorded:

1. number of transitions [n] as anxiety-related parameter

2. distance traveled in the dark chamber [cm] as activity-related-parameter

### Example F

### Statistics

Results are analyzed by t-test (two groups) or one way analysis of variance (ANOVA) when several groups are compared. Tukey test is used for individual comparison. P<0.05 is regarded as significant.

### REFERENCES

Antonini I, Franchetti P, Grifantini M, Martelli S. (1976) A new example of intramolecular 1,5-hydrogen transfer during diazotization reactions. J. Org. Chem. 41: 158-159.
Baddeley A (2003). Working memory: looking back and looking forward. Nat Rev Neurosci 4 (10): 829-839.
Beck A T (2008). The evolution of the cognitive model of depression and its neurobiological correlates. Am J Psychiatry 165: 969-977.
Blokland A, Schreiber R, Prickaerts J (2006). Improving memory: a role for phosphodiesterases. Curr. Pharm. Des 12 (20): 2511-2523.
Boess F G, Hendrix M, van der Staay F J, Erb C, Schreiber R, van Staveren W, de Vente J, Prickaerts J, Blokland A, Koenig G (2004) Inhibition of phosphodiesterase 2 increases neuronal cGMP, synaptic plasticity and memory performance. Neuropharmacology 47 (7): 1081-1092.
Bolger G B, Rodgers L, Riggs M (1994). Differential CNS expression of alternative mRNA isoforms of the mammalian genes encoding cAMP-specific phosphodiesterases. Gene 149: 237-44.
Conti M, Jin S L (1999). The molecular biology of cyclic nucleotide phosphodiesterases. Prog Nucleic Acid Res Mol Biol 63: 1-38.
Crawley J N (1985). Exploratory Behavior Models of Anxiety in Mice. Neurosci Biobehav Rev 9: 37-44.
Duman R S (1998). Novel therapeutic approaches beyond the serotonin receptor. Biol Psychiatry 44 (5): 324-335.

Dunkin J J, Leuchter A F, Cook I A, Kasl-Godley J E, Abrams M, Rosenberg-Thompson S (2000). Executive dysfunction predicts nonresponse to fluoxetine in major depression. J Affect Disord 60: 13-23.
ElYacoubi M, Ledent C, Parmentier M, Bertorelli R, Ongini E, Costentin J, and Vaugeois J M (2001). Adenosine A2A receptor antagonists are potential antidepressants: evidence based on pharmacology and A2A receptor knockout mice. Br J Pharmacol 134: 68-77.
Essayan D M (2001). Cyclic nucleotide phosphodiesterases. J Allergy Clin Immunol 108: 671-680.
Gualtieri C T, Johnson L G, Benedict K B (2006). Neurocognition in depression: patients on and off medication versus healthy comparison subjects. J Neuropsychiatry Clin Neurosci 18: 217-225.
Gorlyn M, Keilp J G, Grunebaum M F, Taylor B P, Oquendo M A, Bruder G E, Stewart J W, Zalsman G, Mann J J (2008). Neuropsychological characteristics as predictor of SSRI treatment response in depressed subjects. J Neural Transm 115: 1213-1219.
Kumar S, Kulkarni S K (1996). Influence of antidepressant drugs on learning and memory paradigms in mice. Indian J Exp Biol 34: 431-435.
Lakics et al. Society of Neuroscience 35^{th} Annual Meeting; 2005, Nov. 12-16, Washington DC.

Mandelli L, Serretti A, Colombo C, Florita M, Santoro A, Rossini D, Zanardi R, Smeraldi E (2006). Improvement of cognitive functioning in mood disorder patients with depressive symptomatic recovery durino treatment: an exploratory analysis. Psychiatry Clin Neurosci 60: 598-604.
Masood A, Nadeem A, Mustafa S J, O'Donnell J M (2008). Reversal of oxidative stress-induced anxiety by inhibition of phosphodiesterase-2 in mice. JPET 326: 369-379.
Menniti F S, Faraci W S, Schmidt C J (2006). Phosphodiesterases in the CNS: targets for drug development. Nat Rev Drug Discov 5 (8): 660-670.
Modell J G, Mountz J M, Beresford T P (1990). Basal ganglia/limbic striatal and thalamocortical involvement in craving and loss of control in alcoholism. J Neuropsychiatry Clin Neurosci 2 (2): 123-144.
Nibuya M, Nestler E J, Duman R S (1996). Chronic antidepressant administration increases the expression of cAMP response element binding protein (CREB) in rat hippocampus. J Neurosci 16 (7): 2365-2372.
Paelecke-Habermann Y, Pohl J, Leplow B (2005). Attention and executive functions in remitted major depression patients. J Affect Disord 89: 125-135.
Porsolt R D (1979). Animal model of depression. Biomedicine 30: 139-40.
Prickaerts J, de Vente J, Honig W, Steinbusch H W, Blokland A (2002). cGMP, but not cAMP, in rat hippocampus is involved in early stages of object memory consolidation. Eur J Pharmacol 436 (1-2): 83-87.
Ramanathan M, Kumar S N, Suresh B (2003). Evaluation of cognitive function of fluoxetine, sertraline and tianeptine in isolation and chronic unpredictable mild stress-induced depressive Wistar rats. Indian J Exp Biol 41: 1269-1272.
Rutten K, Prickaerts J, Blokland A (2006a). Rolipram reverses scopolamine-induced and time-dependent memory deficits in object recognition by different mechanisms of action. Neurobiol Learn Mem 85: 132-138.
Rutten K, Prickaerts J, Hendrix M, van der Staay F J, Sik A, Blokland A (2006b). Time-dependent involvement of cAMP and cGMP in consolidation of object memory: studies using selective phosphodiesterase type 2, 4 and 5 inhibitors. Eur J Pharmacol 558:107-112.
Sadchikova E V, et all. (2000). Synthesis of new heteroaromatic systems: naphtha[2,1-e]imidazo[5,1-c]1,2,4-triazines and benz[e]imidazo[5,1-c]-1,2,4-triazines. Chemistry of Heterocyclic Compounds 36: 465-471
Sadchikova E V and Mokrushin V S (2005). Reactivity of diazoles and azolediazonium salts in C-azo coupling reactions. Russian Chemical Bulletin, International Edition 54: 354-365
Shelton R C, Manier D H, Peterson C S, Ellis T C, Sulser F (1999). Cyclic AMP-dependent protein kinase in subtypes of major depression and normal volunteers. Int J Neuropsychopharmacol 2 (3): 187-192.
Simonov A M, Sitkina L M, Pozharskii A F (1967) Unusual reaction of azo-couplimg in the imidazole series. Chemistry & Industry 34: 1454
Simonov A M et all. (1969). Unusual reaction of azo-couplimg in an imidazole series. Formation of a new heterocyclic system of imidazo-1,2,4-benzotriazine. Khimia Geterotsiklicheskikh Soedinienii 5: 916-922
Soderling, S. H. and Beavo, J. A. (2000). Regulation of cAMP and cGMP signaling: new phosphodiesterases and new functions. Curr Opin Cell Biol 12: 174-179.
Valluzi J A, Chan K (2007). Effect of fluoxetine on hippocampal-dependent and hippocampal-independent learning tasks. Behav Pharmacol 18: 507-513.
Van Staveren W C, Steinbusch H W, Markerink-Van Ittersum M, Repaske D R, Goy M F, Kotera J, Omori K, Beavo J A, De Vente J (2003). mRNA expression patterns of the cGMP-hydrolyzing phosphodiesterases types 2, 5, and 9 during development of the rat brain. J Comp Neurol 467: 566-80.

A number of embodiments of the invention have been described. Nevertheless, it will be understood that various modifications may be made without departing from the spirit and scope of the invention.

What is claimed is:

1. A compound of formula (I): 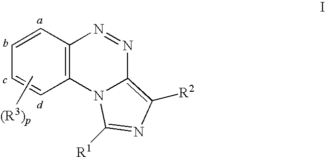
or a pharmaceutically acceptable salt thereof; wherein:
a, b, c, and d indicate four possible positions on the ring for each R³, when present;
p is 0 or an integer from 1 to 4;
R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
any two groups R³ may together be alkylene or alkenylene completing a 3- to 8-membered saturated or unsaturated ring together with the carbon atoms to which they are attached, which ring is unsubstituted or substituted with one or more independently selected Z groups; or
any two groups of R³ may, together with the atoms to which they are attached, form a heterocyclo group which is unsubstituted or substituted with one or more independently selected Z groups;
each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;
each R⁵, R⁶, R⁷, R⁸, R⁹ and R¹⁰ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or
R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or
any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;
each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;
each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;
each Y³ and Y⁴ is independently selected from a single bond, alkylene, alkenylene, and alkynylene; and
each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy;
provided that:
(a) when p is 0, then R¹ and R² are not each H or each methyl;
(b) when R¹ is H; R² is —C(O)O-(ethyl); p is 1; and R³ is at the c position of the ring; then R³ is other than hydroxyl;
(c) when R¹ is H; R² is nitro or —C(O)O-(ethyl); p is 2; and each R³ is at the a and c positions of the ring; then each R³ is other than methoxy;
(d) when R¹ is H; p is 2; each R³ is methyl; and the two R³ groups are at the a and c positions of the ring; then R² is other than —C(O)OH, —C(O)O-(ethyl), or benzylthio;
(e) when R¹ is H; R² is nitro, —C(O)O-(ethyl), —C(O)NH₂, or —CONH-(methyl); p is 1; and R³ is at the b position of the ring, then R³ is other than methyl;
(f) when R¹ is H; R² is —C(O)O-(ethyl); p is 1; and R³ is at the b position of the ring; then R³ is other than amino;
(g) when R¹ is methyl, 1,3-dixolan-2-yl, hydroxymethyl, or formyl; and R² is H, then p is other than 0;
(h) when R¹ is H; R² is H; p is 1; and R³ is at the c position of the ring; then R³ is other than chloro or dimethylamino;
(i) when R¹ is H; p is 1; R³ is hydroxyl; and R³ is at the c position of the ring; then R² other than cyano;
(j) when R² is H; p is 1; R³ is hydroxyl; and R³ is at the c position of the ring; then R¹ other than cyano;
(k) when R¹ is H; R² is H; p is 1; and R³ is at the b position of the ring; then R³ is other than chloro, bromo, methyl, methoxy, nitro, and trifluoromethyl;
(l) when R² is H, p is 1; R³ is methoxy; and R³ is at the b position of the ring; then R¹ is other than methyl, ethyl, methoxy, ethoxy, trifluoromethyl or phenyl;
(m) when R¹ is H, p is 1; R³ is methoxy; and R³ is at the b position of the ring; then R² is other than methyl, ethyl, methoxy, ethoxy, trifluoromethyl or phenyl;
(n) when R² is H, p is 0 or 1; R³ is chloro (when p is 1); and R³ is at the b position of the ring (when p is 1); then R¹ is other than chloro, cyano, nitro, methyl, ethyl, isopropyl, methoxy, ethoxy, trifluoromethyl, phenyl, methylthio, —C(O)O-(methyl), —C(O)-(methyl), —C(O)N-(methyl)₂, —N(methyl)₂, or benzyl;
(o) when R¹ is H, p is 0 or 1; R³ is chloro (when p is 1); and R³ is at the b position of the ring (when p is 1); then R² is other than chloro, cyano, nitro, methyl, ethyl, isopropyl, methoxy, ethoxy, trifluoromethyl, phenyl, methylthio, —C(O)O-(methyl), —C(O)O-(ethyl), —C(O)-(methyl), —C(O)N-(methyl)₂, —N(methyl)₂, or benzyl;
(p) when R¹ is H; p is 2; each R³ is chloro; and the two R³ groups are at the b and d positions of the ring; then R² is other than H;
(q) when two R³ groups, together with the atoms to which they are attached, form an unsubstituted benzene ring, and R¹ is H, then R² is other than hydrogen, nitro, —C(O)O-(ethyl), —C(O)NHNH₂, —C(O)NH₂, —C(O)NH-(methyl), —C(O)NH-(phenyl), —C(O)NH-(cyclohexyl), —C(O)NH-(morpholin-1-yl), —C(O)NH-(piperidin-1-yl), —C(O)NH-(4-methylphenyl), —C(O)NH-(4-chlorophenyl), and —NH—C(O)O-(ethyl); and
(r) when two R³ groups, together with the atoms to which they are attached, form an heterocyclo ring, that heterocyclo ring is other than a 12-membered ring with 4 or more oxygen atoms.

2. The compound of claim 1, or a pharmaceutically acceptable salt thereof, wherein p is 1, 2, or 3; R¹ and R² are other than H; and each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂.

3. The compound of claim 1, or a pharmaceutically acceptable salt thereof, wherein p is 1, 2, or 3.

4. The compound of claim 1, or a pharmaceutically acceptable salt thereof, wherein p is 1 or 2.

5. The compound of claim 1, or a pharmaceutically acceptable salt thereof, wherein p is 1.

6. The compound of claim 1, or a pharmaceutically acceptable salt thereof, wherein p is 2.

7. The compound of claim 1, or a pharmaceutically acceptable salt thereof, wherein R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups.

8. The compound of claim 1, or a pharmaceutically acceptable salt thereof, wherein R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups; and wherein each R⁵, R⁶, R⁸, R⁹, and R¹⁰ is independently selected from H, alkyl, and haloalkyl.

9. The compound of claim 1, or a pharmaceutically acceptable salt thereof, wherein R¹ is selected from halo, alkyl, cycloalkyl, aryl, and heterocyclo, wherein said alkyl, cycloalkyl, aryl, and heterocyclo are each unsubstituted or substituted with one or more independently selected Z groups.

10. The compound of claim 1, or a pharmaceutically acceptable salt thereof, wherein R¹ is selected from alkyl, cycloalkyl, aryl, and heterocyclo, wherein said alkyl, cycloalkyl, aryl, and heterocyclo are each unsubstituted or substituted with one or more independently selected Z groups.

11. The compound of claim 1, or a pharmaceutically acceptable salt thereof, wherein R¹ is selected from alkyl, wherein said alkyl is unsubstituted or substituted with one or more independently selected Z groups.

12. The compound of claim 1, or a pharmaceutically acceptable salt thereof, wherein R¹ is selected from cycloalkyl, wherein said cycloalkyl is unsubstituted or substituted with one or more independently selected Z groups.

13. The compound of claim 1, or a pharmaceutically acceptable salt thereof, wherein R¹ is selected from aryl and heteroaryl, wherein said aryl and heteroaryl are each unsubstituted or substituted with one or more independently selected Z groups.

14. The compound of claim 1, or a pharmaceutically acceptable salt thereof, wherein R¹ is heterocyclo, which is unsubstituted or substituted with one or more independently selected Z groups.

15. The compound of claim 1, or a pharmaceutically acceptable salt thereof, wherein R¹ is heteroaryl, which is unsubstituted or substituted with one or more independently selected Z groups.

16. The compound of claim 1, or a pharmaceutically acceptable salt thereof, wherein R¹ is aryl, which is unsubstituted or substituted with one or more independently selected Z groups.

17. The compound of claim 1, or a pharmaceutically acceptable salt thereof, wherein R¹ is selected from bromo, ethyl, propyl, isobutyl, cyclohexyl, a phenyl ring, a thiophene ring, a pyrazole ring, an isooxazole ring, a thiazole ring, and a pyridine ring; wherein said ethyl, propyl, isobutyl, cyclohexyl, a phenyl ring, a thiophene ring, a pyrazole ring, an isooxazole ring, a thiazole ring, and a pyridine ring are each unsubstituted or substituted with one or more independently selected Z groups.

18. The compound of claim 1, or a pharmaceutically acceptable salt thereof, wherein R¹ is selected from H, bromo, ethyl, propyl, isobutyl, cyclohexyl, phenyl, thiophen-2-yl, 1H-pyrazol-5-yl, 1H-pyrazol-4-yl, furan-3-yl, isooxazol-4-yl, thiazol-5-yl, pyridin-2-yl, pyridin-3-yl, and pyridin-4-yl; wherein said ethyl, propyl, isobutyl, cyclohexyl, phenyl, thiophen-2-yl, 1H-pyrazol-5-yl, 1H-pyrazol-4-yl, isooxazol-4-yl, thiazol-5-yl, pyridin-2-yl, pyridin-3-yl, and pyridin-4-yl are each unsubstituted or substituted with one or more independently selected Z groups.

19. The compound of claim 1, or a pharmaceutically acceptable salt thereof, wherein R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups.

20. The compound of claim 1, or a pharmaceutically acceptable salt thereof, wherein R² is alkyl.

21. The compound of claim 1, or a pharmaceutically acceptable salt thereof, wherein R² is methyl.

22. The compound of claim 1, or a pharmaceutically acceptable salt thereof, wherein each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups.

23. The compound of claim 1, or a pharmaceutically acceptable salt thereof, wherein each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups; and wherein each R⁵, R⁶, R⁸, R⁹, and R¹⁰ is independently selected from hydrogen, alkyl, and haloalkyl.

24. The compound of claim 1, or a pharmaceutically acceptable salt thereof, wherein each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups.

25. The compound of claim 1, or a pharmaceutically acceptable salt thereof, wherein each R³ is independently selected from halo, alkyl, —OH, —OR⁴, aryl, and heterocyclo, wherein said alkyl, aryl, and heterocyclo are each unsubstituted or substituted with one or more independently selected Z groups; and wherein each R⁴ is independently alkyl, haloalkyl, cycloalkylalkyl or aralkyl.

26. The compound of claim 1, or a pharmaceutically acceptable salt thereof, wherein each R³ is independently selected from chloro, fluoro, —OH, trifluoromethyl, methoxy, difluoromethoxy, cyclopropylmethoxy, piperidinyl, morpholinyl, piperazinyl, phenyl, 1H-imidazol-1-yl, and benzyloxy; wherein said piperidinyl, morpholinyl, piperazinyl, phenyl, and 1H-imidazol-1-yl are are each unsubstituted or substituted with one or more independently selected Z groups.

27. The compound of claim 1, or a pharmaceutically acceptable salt thereof, wherein each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo.

28. The compound of claim 1, or a pharmaceutically acceptable salt thereof, wherein each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein each R¹¹ is independently alkyl or haloalkyl; wherein each R¹², R¹³, R¹⁴, R¹⁵, R¹⁶, and R¹⁷ is independently selected from hydrogen, alkyl, and haloalkyl; and wherein said alkyl, cycloalkyl, cycloalkylalkyl, aryl, aralkyl, heterocyclo, and heterocycloalkyl is unsubstituted or substituted with one or more independently selected Z¹ groups.

29. The compound of claim 1, or a pharmaceutically acceptable salt thereof, wherein each Z is independently selected from halo, cyano, nitro, alkyl, haloalkyl, cycloalkyl, —OH, —OR¹¹, —SH, —SR¹¹, — C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein each R¹¹ is independently alkyl or haloalkyl; and wherein each R¹², R¹³, R¹⁴, R¹⁵, R¹⁶, and R¹⁷ is independently selected from H, alkyl, and haloalkyl.

30. The compound of claim 1, or a pharmaceutically acceptable salt thereof, wherein each Z is independently selected from halo, cyano, nitro, alkyl, haloalkyl, alkoxy, haloalkoxy, cycloalkyl, —OH, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo; wherein each R¹¹ is independently alkyl or haloalkyl; and wherein each R¹², R¹³, and R¹⁷ is independently selected from H and alkyl.

31. The compound of claim 7, or a pharmaceutically acceptable salt thereof, wherein each Z group is independently selected from halo, alkyl, haloalkyl, alkoxy, haloalkoxy, and —C(O)NR¹²R¹³.

32. The compound of claim 7, or a pharmaceutically acceptable salt thereof, wherein each Z group is independently selected from chloro, fluoro, methyl, isopropyl, trifluoromethyl, methoxy, ethoxy, isoproxy, n-propoxy, butoxy, trifluoromethoxy, and —C(O)NH₂.

33. The compound of claim 22, or a pharmaceutically acceptable salt thereof, wherein each Z is independently selected from halo, alkyl, haloalkyl, alkoxy, and cycloalkyl.

34. The compound of claim 22, or a pharmaceutically acceptable salt thereof, wherein each Z is independently selected from chloro, fluoro, methyl, isobutyl, trifluoromethyl, ethoxy, propoxy, butoxy, and cyclohexyl.

35. The compound of claim 1, or a pharmaceutically acceptable salt thereof, wherein:
p is 1, 2, or 3;
R¹ is selected from alkyl, aryl, aralkyl or heterocyclo, unsubstituted or substituted with one to three independently selected Z groups;
R² is selected from alkyl; and
each R³ is independently selected from —OH, —OR⁴, halo, cyano, nitro and —NR⁵R⁶, wherein Y¹ represents a single bond.

36. The compound of claim 1, or a pharmaceutically acceptable salt thereof, wherein:
p is 1, 2, or 3;
R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups;
R² is selected from alkyl, cycloalkyl, cycloalkylalkyl, heterocyclo, and heterocycloalkyl; wherein alkyl, cycloalkyl, cycloalkylalkyl, heterocyclo, and heterocycloalkyl are each unsubstituted or substituted with one or more independently selected Z groups;
each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups; and
each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein said alkyl, cycloalkyl, cycloalkylalkyl, aryl, aralkyl, heterocyclo, and heterocycloalkyl is unsubstituted or substituted with one or more independently selected Z¹ groups.

37. The compound of claim 1, or a pharmaceutically acceptable salt thereof, wherein:
p is 1, 2, or 3;
R¹ is selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups;
R² is selected from alkyl;
each R³ is independently selected from alkyl, cycloalkyl, cycloalkylalkyl, aryl, aralkyl, heterocyclo, heterocycloalkyl, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —NR⁵R⁶, —C(O)NR⁵R⁶, —S(O)₂—NR⁵R⁶, —N(R⁷)—C(O)—NR⁸R⁹, —N(R¹⁰)—C(O)—R⁴, and —N(R¹⁰)—C(O)O—R⁴; wherein said alkyl, cycloalkyl, cycloalkylalkyl, aryl, aralkyl, heterocyclo, heterocycloalkyl are each unsubstituted or substituted by one or more independently selected Z groups;
each Z group is independently selected from alkyl, cycloalkyl, cycloalkylalkyl, aryl, aralkyl, heterocyclo, heterocycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo; wherein said alkyl, cycloalkyl, cycloalkylalkyl, aryl, aralkyl, heterocyclo, and heterocycloalkyl is unsubstituted or substituted with one or more independently selected Z¹ groups;
each R¹¹ is independently alkyl or haloalkyl; and
each R⁵, R⁶, R⁸, R⁹, R¹⁰, R¹², R¹³, R¹⁴, R¹⁵, R¹⁶, and R¹⁷ is independently selected from H, alkyl, and haloalkyl.

38. The compound of claim 1, or a pharmaceutically acceptable salt thereof, wherein:
p is 1, 2, or 3;
R¹ is selected from halo, alkyl, cycloalkyl, aryl, and heteroaryl, wherein said alkyl, cycloalkyl, aryl, and heteroaryl are each unsubstituted or substituted with one or more independently selected Z groups;
R² is selected from alkyl;
each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;
each Z is independently selected from halo, cyano, nitro, alkyl, haloalkyl, alkoxy, haloalkoxy, cycloalkyl, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —NR¹²R¹³, —C(O)—NR¹²R¹³, —S(O)₂—NR¹²R¹³, —OC(O)—NR¹²R¹³, —N(R¹⁴)—C(O)—NR¹⁵R¹⁶, —N(R¹⁷)—C(O)—R¹¹, —N(R¹⁷)—C(O)O—R¹¹, and oxo;
each R¹¹ is independently alkyl or haloalkyl; and
each R¹², R¹³, R¹⁴, R¹⁵, R¹⁶, and R¹⁷ is independently selected from H, alkyl, and haloalkyl.

39. The compound of claim 1, or a pharmaceutically acceptable salt thereof, wherein:
p is 1, 2, or 3;
R¹ is selected from alkyl, cycloalkyl, aryl, and heteroaryl, wherein said alkyl, cycloalkyl, aryl, and heteroaryl are each unsubstituted or substituted with one or more independently selected Z groups;
R² is selected from alkyl;
each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;
each Z is independently selected from halo, cyano, nitro, alkyl, haloalkyl, alkoxy, haloalkoxy, cycloalkyl, —OH, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo;
each R¹¹ is independently alkyl or haloalkyl; and
each R¹², R¹³, and R¹⁷ is independently selected from H, alkyl, and haloalkyl.

40. The compound of claim 1, or a pharmaceutically acceptable salt thereof, wherein:
p is 1, 2, or 3;
R¹ is alkyl or cycloalkyl, which are each unsubstituted or substituted with one or more independently selected Z groups;
R² is selected from alkyl;
each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;
each Z is independently selected from halo, cyano, nitro, alkyl, haloalkyl, alkoxy, haloalkoxy, cycloalkyl, —OH, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo;
each R¹¹ is independently alkyl or haloalkyl; and
each R¹², R¹³, and R¹⁷ is independently selected from H, alkyl, and haloalkyl.

41. The compound of claim 1, or a pharmaceutically acceptable salt thereof, wherein:
p is 1, 2, or 3;
R¹ is aryl or heteroaryl, which are each unsubstituted or substituted with one or more independently selected Z groups;
R² is selected from alkyl;
each R³ is independently selected from halo, cyano, nitro, —OH, —OR⁴, alkyl, cycloalkyl, cycloalkylalkyl, aryl, aralkyl, heterocyclo, and heterocycloalkyl, wherein said alkyl, cycloalkyl, cycloalkylalkyl, aryl, aralkyl, heterocyclo, and heterocycloalkyl are each unsubstituted or substituted with one or more independently selected Z groups;
each Z is independently selected from halo, cyano, nitro, alkyl, haloalkyl, alkoxy, haloalkoxy, cycloalkyl, —OH, —OR¹¹, —NR¹²R¹³, —C(O)—NR¹²R¹³, —S(O)₂—NR¹²R¹³, —OC(O)—NR¹²R¹³, and —N(R¹⁷)—C(O)—R¹¹, and oxo;
each R¹¹ is independently alkyl or haloalkyl; and
each R¹², R¹³, and R¹⁷ is independently selected from H, alkyl, and haloalkyl.

42. The compound of claim 1, or a pharmaceutically acceptable salt thereof, wherein:
p is 1, 2, or 3;
R¹ is selected from H, bromo, ethyl, propyl, isobutyl, cyclohexyl, a phenyl ring, a thiophene ring, a pyrazole ring, an isooxazole ring, a thiazole ring, and a pyridine ring; wherein said ethyl, propyl, isobutyl, cyclohexyl, a phenyl ring, a thiophene ring, a pyrazole ring, an isooxazole ring, a thiazole ring, and a pyridine ring are each unsubstituted or substituted with one or more Z groups independently selected from halo, alkyl, haloalkyl, alkoxy, haloalkoxy, and —C(O)NR¹²R¹³.
R² is selected from alkyl;
each R³ is independently selected from halo, alkyl, —OH, —OR⁴, aryl, and heterocyclo, wherein said alkyl, aryl, and heterocyclo are each unsubstituted or substituted with one or more Z groups independently selected from halo, alkyl, haloalkyl, alkoxy, and cycloalkyl;
each R¹² and R¹³ is independently selected from H and alkyl.

43. The compound of claim 1, or a pharmaceutically acceptable salt thereof, wherein:
p is 1, 2, or 3;
R¹ is selected from H, bromo, ethyl, propyl, isobutyl, cyclohexyl, phenyl, thiophen-2-yl, 1H-pyrazol-5-yl, 1H-pyrazol-4-yl, isooxazol-4-yl, thiazol-5-yl, pyridin-2-yl, pyridin-3-yl, and pyridin-4-yl; wherein said ethyl, propyl, isobutyl, cyclohexyl, phenyl, thiophen-2-yl, 1H-pyrazol-5-yl, 1H-pyrazol-4-yl, isooxazol-4-yl, thiazol-5-yl, pyridin-2-yl, pyridin-3-yl, and pyridin-4-yl are each unsubstituted or substituted with one or more groups independently selected from chloro, fluoro, methyl, isopropyl, trifluoromethyl, methoxy, ethoxy, isoproxy, n-propoxy, butoxy, trifluoromethoxy, and —C(O)NH₂;
R² is methyl; and
each R³ is independently selected from chloro, fluoro, —OH, trifluoromethyl, methoxy, difluoromethoxy, cyclopropylmethoxy, piperidinyl, morpholinyl, piperazinyl, phenyl, 1H-imidazol-1-yl, and benzyloxy; wherein said piperidinyl, morpholinyl, piperazinyl, phenyl, and 1H-imidazol-1-yl are are each unsubstituted or substituted with one or more groups independently selected from chloro, fluoro, methyl, isobutyl, trifluoromethyl, ethoxy, propoxy, butoxy, and cyclohexyl.

44. The compound of claim 1, wherein said compound is a compound of Formula Ib: 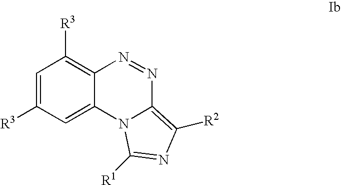
or a pharmaceutically acceptable salt thereof.

45. The compound of claim 1, wherein said compound is a compound of Formula Ic: 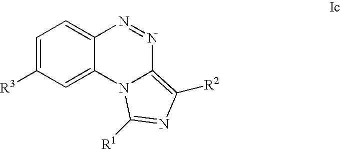
or a pharmaceutically acceptable salt thereof.

46. The compound of claim 1, selected from:
8-fluoro-3-methyl-1-propyl-imidazo[5,1-c][1,2,4]benzotriazine;
7-methoxy-3-methyl-1-propyl-imidazo[5,1-c][1,2,4]benzotriazine;
1-ethyl-8-fluoro-3-methyl-imidazo[5,1-c][1,2,4]benzotriazine;
1-cyclohexyl-8-fluoro-3-methyl-imidazo[5,1-c][1,2,4]benzotriazine;
1-(2,5-dichlorophenyl)-8-methoxy-3-methyl-imidazo[5,1-c][1,2,4]benzotriazine;
1-(2,5-dichlorophenyl)-7-fluoro-3-methyl-imidazo[5,1-c][1,2,4]benzotriazine; and
1-(2,5-dichlorophenyl)-7-methoxy-3-methyl-imidazo[5,1-c][1,2,4]benzotriazine;
or a pharmaceutically acceptable salt thereof.

47. The compound of claim 1, selected from:
1-(2,5-dichlorophenyl)-8-fluoro-3-methylbenzo[e]imidazo[5,1-c][1,2,4]triazine;
8-fluoro-1-isobutyl-3-methylbenzo[e]imidazo[5,1-c][1,2,4]triazine;
1-sec-butyl-8-fluoro-3-methylbenzo[e]imidazo[5,1-c][1,2,4]triazine;
8-fluoro-3-methylbenzo[e]imidazo[5,1-c][1,2,4]triazine;
8-fluoro-3-methyl-1-(2-methylpyridin-3-yl)benzo[e]imidazo[5,1-c][1,2,4]triazine;
1-(2-chlorophenyl)-8-fluoro-3-methylbenzo[e]imidazo[5,1-c][1,2,4]triazine;
1-(2,3-dichlorophenyl)-8-fluoro-3-methylbenzo[e]imidazo[5,1-c][1,2,4]triazine;
8-fluoro-3-methyl-1-(1-methyl-1H-pyrazol-5-yl)benzo[e]imidazo[5,1-c][1,2,4]triazine;
8-fluoro-1-(2-methoxyphenyl)-3-methylbenzo[e]imidazo[5,1-c][1,2,4]triazine;
8-fluoro-3-methyl-1-o-tolylbenzo[e]imidazo[5,1-c][1,2,4]triazine;
1-(2-chloro-5-methoxyphenyl)-8-fluoro-3-methylbenzo[e]imidazo[5,1-c][1,2,4]triazine;
1-(4-chloropyridin-3-yl)-8-fluoro-3-methylbenzo[e]imidazo[5,1-c][1,2,4]triazine;
8-fluoro-1-(2-fluoro-5-isopropoxyphenyl)-3-methylbenzo[e]imidazo[5,1-c][1,2,4]triazine;
8-fluoro-1-(2-fluoro-5-(trifluoromethyl)phenyl)-3-methylbenzo[e]imidazo[5,1-c][1,2,4]triazine;
1-(5-butoxy-2-fluorophenyl)-8-fluoro-3-methylbenzo[e]imidazo[5,1-c][1,2,4]triazine;
8-fluoro-1-(2-fluoro-5-propoxyphenyl)-3-methylbenzo[e]imidazo[5,1-c][1,2,4]triazine;
1-(5-ethoxy-2-fluorophenyl)-8-fluoro-3-methylbenzo[e]imidazo[5,1-c][1,2,4]triazine;
7-fluoro-3-methyl-1-(2-methylpyridin-3-yl)benzo[e]imidazo[5,1-c][1,2,4]triazine;
7-fluoro-1-(2-methoxyphenyl)-3-methylbenzo[e]imidazo[5,1-c][1,2,4]triazine;
7-fluoro-1-(4-fluoro-2-methylphenyl)-3-methylbenzo[e]imidazo[5,1-c][1,2,4]triazine;
7-fluoro-3-methyl-1-o-tolylbenzo[e]imidazo[5,1-c][1,2,4]triazine;
1-(2-chloro-5-methoxyphenyl)-7-fluoro-3-methylbenzo[e]imidazo[5,1-c][1,2,4]triazine;
7-fluoro-1-(2-fluoro-5-(trifluoromethyl)phenyl)-3-methylbenzo[e]imidazo[5,1-c][1,2,4]triazine;
7-fluoro-1-(2-fluoro-5-isopropoxyphenyl)-3-methylbenzo[e]imidazo[5,1-c][1,2,4]triazine;
1-(5-butoxy-2-fluorophenyl)-7-fluoro-3-methylbenzo[e]imidazo[5,1-c][1,2,4]triazine;
7-fluoro-1-(2-fluoro-5-propoxyphenyl)-3-methylbenzo[e]imidazo[5,1-c][1,2,4]triazine;
1-(2-chlorophenyl)-7-methoxy-3-methylbenzo[e]imidazo[5,1-c][1,2,4]triazine;
7-methoxy-3-methylbenzo[e]imidazo[5,1-c][1,2,4]triazine;
7-methoxy-3-methyl-1-(2-methylpyridin-3-yl)benzo[e]imidazo[5,1-c][1,2,4]triazine;
8-chloro-1-(2,5-dichlorophenyl)-3-methylbenzo[e]imidazo[5,1-c][1,2,4]triazine;
8-chloro-3-methyl-1-(2-methylpyridin-3-yl)benzo[e]imidazo[5,1-c][1,2,4]triazine;
8-chloro-1-(2-methoxyphenyl)-3-methylbenzo[e]imidazo[5,1-c][1,2,4]triazine;
8-chloro-3-methyl-1-(1-methyl-1H-pyrazol-5-yl)benzo[e]imidazo[5,1-c][1,2,4]triazine;
2-(8-chloro-3-methylbenzo[e]imidazo[5,1-c][1,2,4]triazin-1-yl)benzamide;
8-chloro-3-methylbenzo[e]imidazo[5,1-c][1,2,4]triazine;
8-chloro-1-(2-fluoro-5-isopropoxyphenyl)-3-methylbenzo[e]imidazo[5,1-c][1,2,4]triazine;
1-(2-fluoro-5-isopropoxyphenyl)-8-methoxy-3-methylbenzo[e]imidazo[5,1-c][1,2,4]triazine;
6,8-dimethoxy-3-methyl-1-(4-methylpyridin-3-yl)imidazo[5,1-c][1,2,4]benzotriazine;
1-(2-chlorophenyl)-7,8-dimethoxy-3-methylbenzo[e]imidazo[5,1-c][1,2,4]triazine;
6,8-dimethoxy-3-methyl-1-(3-methylpyridin-4-yl)imidazo[5,1-c][1,2,4]benzotriazine;
6,8-dimethoxy-3-methyl-1-(2-methylpyridin-3-yl)imidazo[5,1-c][1,2,4]benzotriazine;
7,8-dimethoxy-3-methyl-1-o-tolylbenzo[e]imidazo[5,1-c][1,2,4]triazine;
7,8-dimethoxy-3-methyl-1-(pyridin-2-yl)benzo[e]imidazo[5,1-c][1,2,4]triazine;
1-(3,5-dimethyl-1H-pyrazol-4-yl)-6,8-dimethoxy-3-methylimidazo[5,1-c][1,2,4]benzotriazine;
6,8-dimethoxy-3-methyl-1-(1,3,5-trimethyl-1H-pyrazol-4-yl)imidazo[5,1-c][1,2,4]benzotriazine;
1-isobutyl-7,8-dimethoxy-3-methylbenzo[e]imidazo[5,1-c][1,2,4]triazine;
1-bromo-7,8-dimethoxy-3-methylbenzo[e]imidazo[5,1-c][1,2,4]triazine;
1-(2,5-dichlorophenyl)-7,8-dimethoxy-3-methylbenzo[e]imidazo[5,1-c][1,2,4]triazine;
1-(2-chloro-5-methylphenyl)-7,8-dimethoxy-3-methylbenzo[e]imidazo[5,1-c][1,2,4]triazine;
7,8-dimethoxy-3-methyl-1-(2-(trifluoromethyl)phenyl)benzo[e]imidazo[5,1-c][1,2,4]triazine;
1-(2-chlorophenyl)-7,8-difluoro-3-methylbenzo[e]imidazo[5,1-c][1,2,4]triazine;
7,8-difluoro-1-isobutyl-3-methylbenzo[e]imidazo[5,1-c][1,2,4]triazine;
6-methoxy-3-methyl-1-(2-methylphenyl)imidazo[5,1-c][1,2,4]benzotriazine;
1-(2-chlorophenyl)-6-methoxy-3-methylbenzo[e]imidazo[5,1-c][1,2,4]triazine;
6-methoxy-3-methyl-1-(3-methylpyridin-4-yl)imidazo[5,1-c][1,2,4]benzotriazine;
6-methoxy-3-methyl-1-(3-methylpyridin-4-yl)-8-morpholin-4-ylimidazo[5,1-c][1,2,4]benzotriazine;
6-methoxy-3-methyl-1-(2-methylpyridin-3-yl)-8-morpholin-4-ylimidazo[5,1-c][1,2,4]benzotriazine
1-(2-chlorophenyl)-6-methoxy-3-methyl-8-morpholin-4-ylimidazo[5,1-c][1,2,4]benzotriazine;
6-methoxy-3-methyl-1-(2-methylphenyl)-8-morpholin-4-ylimidazo[5,1-c][1,2,4]benzotriazine;
6-fluoro-8-methoxy-1-(3-methoxyphenyl)-3-methylimidazo[5,1-c][1,2,4]benzotriazine;
1-(5-chloro-2-methoxyphenyl)-6-fluoro-8-methoxy-3-methylimidazo[5,1-c][1,2,4]benzotriazine;
6-fluoro-1-(4-fluoro-2-methylphenyl)-8-methoxy-3-methylimidazo[5,1-c][1,2,4]benzotriazine;
1-(2-chloro-4-fluorophenyl)-6-fluoro-8-methoxy-3-methylimidazo[5,1-c][1,2,4]benzotriazine;
1-(2-chloro-4-methylphenyl)-6-fluoro-8-methoxy-3-methylimidazo[5,1-c][1,2,4]benzotriazine;
1-(2-chloro-5-methylphenyl)-6-fluoro-8-methoxy-3-methylimidazo[5,1-c][1,2,4]benzotriazine;
6-fluoro-1-(5-fluoro-2-methylphenyl)-8-methoxy-3-methylimidazo[5,1-c][1,2,4]benzotriazine;
6-fluoro-1-(2-fluoro-4-methylphenyl)-8-methoxy-3-methylimidazo[5,1-c][1,2,4]benzotriazine;
6-fluoro-1-(2-fluoro-5-methylphenyl)-8-methoxy-3-methylimidazo[5,1-c][1,2,4]benzotriazine;
6-fluoro-1-(2-fluoro-5-methoxyphenyl)-8-methoxy-3-methylimidazo[5,1-c][1,2,4]benzotriazine;
1-(2-chloro-5-ethoxyphenyl)-6-fluoro-8-methoxy-3-methylimidazo[5,1-c][1,2,4]benzotriazine;
1-(2-chloro-5-methoxyphenyl)-6-fluoro-8-methoxy-3-methylimidazo[5,1-c][1,2,4]benzotriazine;
1-(5-chloro-2-methylphenyl)-6-fluoro-8-methoxy-3-methylimidazo[5,1-c][1,2,4]benzotriazine;
1-(2-chloro-5-fluorophenyl)-6-fluoro-8-methoxy-3-methylimidazo[5,1-c][1,2,4]benzotriazine;
6-fluoro-8-methoxy-3-methyl-1-(3-methylthiophen-2-yl)imidazo[5,1-c][1,2,4]benzotriazine;
1-[2-chloro-5-(trifluoromethyl)phenyl]-6-fluoro-8-methoxy-3-methylimidazo[5,1-c][1,2,4]benzotriazine;
1-[2-chloro-5-(trifluoromethoxy)phenyl]-6-fluoro-8-methoxy-3-methylimidazo[5,1-c][1,2,4]benzotriazine;
6-fluoro-1-(3-fluoro-2-methylphenyl)-8-methoxy-3-methylimidazo[5,1-c][1,2,4]benzotriazine;
6-fluoro-8-methoxy-3-methyl-1-(1,3,5-trimethyl-1H-pyrazol-4-yl)imidazo[5,1-c][1,2,4]benzotriazine;
6-fluoro-8-methoxy-1-(2-methoxyphenyl)-3-methylimidazo[5,1-c][1,2,4]benzotriazine;
6-fluoro-8-methoxy-3-methyl-1-pyridin-4-ylimidazo[5,1-c][1,2,4]benzotriazine;
1-(5-chloro-2-fluorophenyl)-6-fluoro-8-methoxy-3-methylimidazo[5,1-c][1,2,4]benzotriazine;
1-(3,5-dimethylisoxazol-4-yl)-6-fluoro-8-methoxy-3-methylimidazo[5,1-c][1,2,4]benzotriazine;
6-fluoro-8-methoxy-3-methyl-1-pyridin-3-ylimidazo[5,1-c][1,2,4]benzotriazine;
1-(2,4-dimethyl-1,3-thiazol-5-yl)-6-fluoro-8-methoxy-3-methylimidazo[5,1-c][1,2,4]benzotriazine;
6-fluoro-1-(6-fluoro-5-methylpyridin-3-yl)-8-methoxy-3-methylimidazo[5,1-c][1,2,4]benzotriazine;
6-fluoro-1-(6-fluoro-2-methylpyridin-3-yl)-8-methoxy-3-methylimidazo[5,1-c][1,2,4]benzotriazine;
6-fluoro-1-(2-fluoropyridin-3-yl)-8-methoxy-3-methylimidazo[5,1-c][1,2,4]benzotriazine;
6-fluoro-8-methoxy-1-(5-methoxypyridin-3-yl)-3-methylimidazo[5,1-c][1,2,4]benzotriazine;
1-(3,5-dimethyl-1H-pyrazol-4-yl)-6-fluoro-8-methoxy-3-methylimidazo[5,1-c][1,2,4]benzotriazine;
6-fluoro-8-methoxy-3-methyl-1-(4-methylpyridin-3-yl)imidazo[5,1-c][1,2,4]benzotriazine;
4-fluoro-3-(6-fluoro-8-methoxy-3-methylimidazo[5,1-c][1,2,4]benzotriazin-1-yl)benzamide;
8-fluoro-6-methoxy-1-(3-methoxyphenyl)-3-methylimidazo[5,1-c][1,2,4]benzotriazine;
8-fluoro-6-methoxy-1-(2-methoxyphenyl)-3-methylimidazo[5,1-c][1,2,4]benzotriazine;
1-[2-chloro-5-(trifluoromethyl)phenyl]-8-fluoro-6-methoxy-3-methylimidazo[5,1-c][1,2,4]benzotriazine;
1-(5-chloro-2-methoxyphenyl)-8-fluoro-6-methoxy-3-methylimidazo[5,1-c][1,2,4]benzotriazine;
8-fluoro-1-(5-fluoro-2-methylphenyl)-6-methoxy-3-methylimidazo[5,1-c][1,2,4]benzotriazine;
1-(5-chloro-2-methylphenyl)-8-fluoro-6-methoxy-3-methylimidazo[5,1-c][1,2,4]benzotriazine;
8-fluoro-1-(2-fluoro-5-methylphenyl)-6-methoxy-3-methylimidazo[5,1-c][1,2,4]benzotriazine;
1-(5-chloro-2-fluorophenyl)-8-fluoro-6-methoxy-3-methylimidazo[5,1-c][1,2,4]benzotriazine;
1-(2-chloro-5-methylphenyl)-8-fluoro-6-methoxy-3-methylimidazo[5,1-c][1,2,4]benzotriazine;
1-(2-chloro-5-ethoxyphenyl)-8-fluoro-6-methoxy-3-methylimidazo[5,1-c][1,2,4]benzotriazine;
8-fluoro-6-methoxy-3-methyl-1-pyridin-3-ylimidazo[5,1-c][1,2,4]benzotriazine;
8-fluoro-1-(4-fluoro-2-methylphenyl)-6-methoxy-3-methylimidazo[5,1-c][1,2,4]benzotriazine;
1-(2-chloro-4-fluorophenyl)-8-fluoro-6-methoxy-3-methylimidazo[5,1-c][1,2,4]benzotriazine;
1-(2-chloro-4-methylphenyl)-8-fluoro-6-methoxy-3-methylimidazo[5,1-c][1,2,4]benzotriazine;
8-fluoro-1-(2-fluoro-5-methoxyphenyl)-6-methoxy-3-methylimidazo[5,1-c][1,2,4]benzotriazine;
8-fluoro-1-(2-fluoro-4-methylphenyl)-6-methoxy-3-methylimidazo[5,1-c][1,2,4]benzotriazine;
1-(3,5-dimethylisoxazol-4-yl)-8-fluoro-6-methoxy-3-methylimidazo[5,1-c][1,2,4]benzotriazine;
8-fluoro-6-methoxy-3-methyl-1-(1,3,5-trimethyl-1H-pyrazol-4-yl)imidazo[5,1-c][1,2,4]benzotriazine;
8-fluoro-1-(3-fluoro-2-methylphenyl)-6-methoxy-3-methylimidazo[5,1-c][1,2,4]benzotriazine;
1-(2-chloro-5-fluorophenyl)-8-fluoro-6-methoxy-3-methylimidazo[5,1-c][1,2,4]benzotriazine;
8-fluoro-6-methoxy-3-methyl-1-(3-methylthiophen-2-yl)imidazo[5,1-c][1,2,4]benzotriazine;
8-fluoro-1-(6-fluoro-2-methylpyridin-3-yl)-6-methoxy-3-methylimidazo[5,1-c][1,2,4]benzotriazine;
8-fluoro-1-(6-fluoro-5-methylpyridin-3-yl)-6-methoxy-3-methylimidazo[5,1-c][1,2,4]benzotriazine;
8-fluoro-6-methoxy-1-(5-methoxypyridin-3-yl)-3-methylimidazo[5,1-c][1,2,4]benzotriazine;
8-fluoro-6-methoxy-3-methyl-1-(4-methylpyridin-3-yl)imidazo[5,1-c][1,2,4]benzotriazine;
1-(3,5-dimethyl-1H-pyrazol-4-yl)-8-fluoro-6-methoxy-3-methylimidazo[5,1-c][1,2,4]benzotriazine;
1-[2-chloro-5-(trifluoromethoxy)phenyl]-8-fluoro-6-methoxy-3-methylimidazo[5,1-c][1,2,4]benzotriazine;
1-(2-chloro-5-methoxyphenyl)-8-fluoro-6-methoxy-3-methylimidazo[5,1-c][1,2,4]benzotriazine;
1-(2,4-dimethyl-1,3-thiazol-5-yl)-8-fluoro-6-methoxy-3-methylimidazo[5,1-c][1,2,4]benzotriazine;
4-fluoro-3-(8-fluoro-6-methoxy-3-methylimidazo[5,1-c][1,2,4]benzotriazin-1-yl)benzamide;
8-fluoro-6-methoxy-3-methyl-1-pyridin-4-ylimidazo[5,1-c][1,2,4]benzotriazine;
8-fluoro-6-methoxy-3-methyl-1-(2-methylpyridin-3-yl)imidazo[5,1-c][1,2,4]benzotriazine;
8-fluoro-6-methoxy-3-methyl-1-(3-methylpyridin-4-yl)imidazo[5,1-c][1,2,4]benzotriazine;
6-Chloro-3-methyl-1-(3-methylpyridin-4-yl)-8-(trifluoromethyl)imidazo[5,1-c][1,2,4]benzotriazine;
6-chloro-1-(2,5-dichlorophenyl)-3-methyl-8-(trifluoromethyl)imidazo[5,1-c][1,2,4]benzotriazine;
6-methoxy-3-methyl-1-(3-methylpyridin-4-yl)-8-(trifluoromethyl)benzo[e]imidazo[5,1-c][1,2,4]triazine;
1-(2-Chlorophenyl)-6-methoxy-3-methyl-8-(trifluoromethyl)imidazo[5,1-c][1,2,4]benzotriazine;
6-Methoxy-3-methyl-1-(2-methylpyridin-3-yl)-8-(trifluoromethyl)imidazo[5,1-c][1,2,4]benzotriazine;
6-Methoxy-3-methyl-1-(4-methylpyridin-3-yl)-8-(trifluoromethyl)imidazo[5,1-c][1,2,4]benzotriazine;
6-Methoxy-3-methyl-1-(3-methylthiophen-2-yl)-8-(trifluoromethyl)imidazo[5,1-c][1,2,4]benzotriazine;
6-Methoxy-1-(3-methoxypyridin-4-yl)-3-methyl-8-(trifluoromethyl)benzo[e]imidazo[5,1-c][1,2,4]triazine;
1-(2,5-Dichlorophenyl)-6-methoxy-3-methyl-8-(trifluoromethyl)benzo[e]imidazo[5,1-c][1,2,4]triazine;
1-(3-Fluoro-2-methylphenyl)-6-methoxy-3-methyl-8-(trifluoromethyl)benzo[e]imidazo[5,1-c][1,2,4]triazine;
1-(5-Chloro-2-methoxyphenyl)-6-methoxy-3-methyl-8-(trifluoromethyl)benzo[e]imidazo[5,1-c][1,2,4]triazine;
1-(Furan-3-yl)-6-methoxy-3-methyl-8-(trifluoromethyl)benzo[e]imidazo[5,1-c][1,2,4]triazine;
4-(6-Methoxy-3-methyl-8-(trifluoromethyl)benzo[e]imidazo[5,1-c][1,2,4]triazin-1-yl)-3,5-dimethylisoxazole;
6-Methoxy-3-methyl-1-(thiophen-2-yl)-8-(trifluoromethyl)benzo[e]imidazo[5,1-c][1,2,4]triazine;
1-(5-Fluoro-2-methylphenyl)-6-methoxy-3-methyl-8-(trifluoromethyl)benzo[e]imidazo[5,1-c][1,2,4]triazine;
6-methoxy-3-methyl-1-(4-methylpyridin-3-yl)benzo[e]imidazo[5,1-c][1,2,4]triazin-8-ol;
8-(difluoromethoxy)-6-methoxy-3-methyl-1-(4-methylpyridin-3-yl)benzo[e]imidazo[5,1-c][1,2,4]triazine;
8-(benzyloxy)-6-methoxy-3-methyl-1-(3-methylpyridin-4-yl)imidazo[5,1-c][1,2,4]benzotriazine;
6-methoxy-3-methyl-1-(3-methylpyridin-4-yl)benzo[e]imidazo[5,1-c][1,2,4]triazin-8-ol;
8-(difluoromethoxy)-6-methoxy-3-methyl-1-(3-methylpyridin-4-yl)imidazo[5,1-c][1,2,4]benzotriazine;
8-(benzyloxy)-6-methoxy-3-methyl-1-(2-methylpyridin-3-yl)imidazo[5,1-c][1,2,4]benzotriazine;
6-methoxy-3-methyl-1-(2-methylpyridin-3-yl)imidazo[5,1-c][1,2,4]benzotriazin-8-ol;
8-(difluoromethoxy)-6-methoxy-3-methyl-1-(2-methylpyridin-3-yl)imidazo[5,1-c][1,2,4]benzotriazine;
8-(benzyloxy)-6-methoxy-3-methyl-1-(4-methylpyridin-3-yl)imidazo[5,1-c][1,2,4]benzotriazine;
8-(cyclopropylmethoxy)-6-methoxy-3-methyl-1-(3-methylpyridin-4-yl)benzo[e]imidazo[5,1-c][1,2,4]triazine;
8-(cyclopropylmethoxy)-6-methoxy-3-methyl-1-(2-methylpyridin-3-yl)benzo[e]imidazo[5,1-c][1,2,4]triazine;
8-(difluoromethoxy)-6-methoxy-3-methyl-1-(1,3,5-trimethyl-1H-pyrazol-4-yl)benzo[e]imidazo[5,1-c][1,2,4]triazine;
8-(difluoromethoxy)-1-(3,5-dimethyl-1H-pyrazol-4-yl)-6-methoxy-3-methylbenzo[e]imidazo[5,1-c][1,2,4]triazine;
4-(8-(difluoromethoxy)-6-methoxy-3-methylbenzo[e]imidazo[5,1-c][1,2,4]triazin-1-yl)-3,5-dimethylisoxazole;
5-(8-(difluoromethoxy)-6-methoxy-3-methylbenzo[e]imidazo[5,1-c][1,2,4]triazin-1-yl)-2,4-dimethylthiazole;
8-(difluoromethoxy)-1-(3-fluoro-2-methylphenyl)-6-methoxy-3-methylbenzo[e]imidazo[5,1-c][1,2,4]triazine;
8-(difluoromethoxy)-1-(5-fluoro-2-methylphenyl)-6-methoxy-3-methylbenzo[e]imidazo[5,1-c][1,2,4]triazine;
1-(2-chloro-5-fluorophenyl)-8-(difluoromethoxy)-6-methoxy-3-methylbenzo[e]imidazo[5,1-c][1,2,4]triazine;
1-(5-chloro-2-methoxyphenyl)-8-(difluoromethoxy)-6-methoxy-3-methylbenzo[e]imidazo[5,1-c][1,2,4]triazine;
1-(2,5-dichlorophenyl)-8-(difluoromethoxy)-6-methoxy-3-methylimidazo[5,1-c][1,2,4]benzotriazine;
1-(2-chlorophenyl)-8-(difluoromethoxy)-6-methoxy-3-methylimidazo[5,1-c][1,2,4]benzotriazine;
8-(difluoromethoxy)-6-methoxy-1-(5-methoxypyridin-3-yl)-3-methylimidazo[5,1-c][1,2,4]benzotriazine;
1,8-Bis-(2,5-dichloro-phenyl)-3-methyl-imidazo[5,1-c][1,2,4]benzotriazine;
1,8-Bis-(2-chloro-phenyl)-3-methyl-imidazo[5,1-c][1,2,4]benzotriazine;
1,8-Bis-(2,3-dichloro-phenyl)-3-methyl-imidazo[5,1-c][1,2,4]benzotriazine;
1,8-Bis-(2-fluoro-5-propoxy-phenyl)-3-methyl-imidazo[5,1-c][1,2,4]benzotriazine;
1,8-Bis-(5-butoxy-2-fluoro-phenyl)-3-methyl-imidazo[5,1-c][1,2,4]benzotriazine;
1,8-Bis-(2-fluoro-5-trifluoromethyl-phenyl)-3-methyl-imidazo[5,1-c][1,2,4]benzotriazine;
1,8-Bis-(2-fluoro-5-isopropoxy-phenyl)-3-methyl-imidazo[5,1-c][1,2,4]benzotriazine;
1,8-Bis-(5-ethoxy-2-fluoro-phenyl)-3-methyl-imidazo[5,1-c][1,2,4]benzotriazine;
1-Cyclohexyl-8-(2-cyclohexyl-4-methyl-imidazol-1-yl)-3-methyl-imidazo[5,1-c][1,2,4]benzotriazine;
1-(2,5-Dichloro-phenyl)-3-methyl-8-piperidin-1-yl-imidazo[5,1-c][1,2,4]benzotriazine;
1-(2,5-Dichloro-phenyl)-3-methyl-8-morpholin-4-yl-imidazo[5,1-c][1,2,4]benzotriazine;
1-Isobutyl-8-(2-isobutyl-4-methyl-imidazol-1-yl)-3-methyl-imidazo[5,1-c][1,2,4]benzotriazine;
1-(2-Chloro-phenyl)-3-methyl-8-piperidin-1-yl-imidazo[5,1-c][1,2,4]benzotriazine;
1-(2-Chloro-phenyl)-3-methyl-8-morpholin-4-yl-imidazo[5,1-c][1,2,4]benzotriazine;
1-(2-Chloro-phenyl)-8-imidazol-1-yl-3-methyl-imidazo[5,1-c][1,2,4]benzotriazine;
1-(2-Chloro-phenyl)-3-methyl-8-(4-methyl-piperazin-1-yl)-imidazo[5,1-c][1,2,4]benzotriazine; and
1-(2-Chloro-phenyl)-8-(4-fluoro-benzyloxy)-3-methyl-imidazo[5,1-c][1,2,4]benzotriazine;
or a pharmaceutically acceptable salt thereof.

48. A pharmaceutical composition comprising a compound of claim 1, or a pharmaceutically acceptable salt thereof, and a pharmaceutically acceptable carrier.

49. A method of treating disorders associated with phosphodiesterase 2 or 10 hyperactivity, the method comprising administering to a patient in need thereof a therapeutically effective amount of a compound of claim 1, or a pharmaceutically acceptable salt thereof.

50. A method of treating central nervous system disorders in a patient in need thereof comprising, administering to said patient a therapeutically effective amount of a compound of claim 1, or a pharmaceutically acceptable salt thereof.

51. The method of claim 50, wherein the neurological and psychiatric disorders are selected from mood (affective) disorders; neurotic, stress-related and somatoform disorders; disorders comprising the symptom of cognitive deficiency in a mammal; attention deficit disorders, executive function deficits (working memory deficits), dysfunction of impulse control, extrapyramidal symptoms, and disorders that are based on a malfunction of basal ganglia; behavioural and emotional disorders with onset usually occurring in childhood and adolescence; disorders of psychological development; systemic atrophies primarily affecting the central nervous system; extrapyramidal and movement disorders; behavioural syndromes associated with physiological disturbances and physical factors; disorders of adult personality and behaviour; schizophrenia and other psychotic disorders; mental and behavioural disorders due to psychoactive substance use; sexual dysfunction; mental retardation; factitious disorders; episodic and paroxysmal disorders; epilepsy; narcolepsy; and dementia.

52. The method of claim 51, wherein the mood disorders are selected from bipolar disorder I depressed, hypomanic, manic and mixed form; bipolar disorder II; depressive disorders; depressive episode or recurrent major depressive disorder; minor depressive disorder; depressive disorder with postpartum onset; depressive disorders with psychotic symptoms; cyclothymia; dysthymia, euthymia; and premenstrual dysphoric disorder.

53. The method of claim 51, wherein the neurotic, stress-related and somatoform disorders are selected from anxiety disorders, general anxiety disorder, panic disorder with or without agoraphobia, specific phobia, social phobia, chronic anxiety disorders, obsessive compulsive disorder, post traumatic stress disorder (PTSD), and depersonalisation-derealisation syndrome.

54. The method of claim 51, wherein the symptom cognitive deficits are selected from cognitive deficits related to psychosis, age-associated memory impairment, Parkinson's disease, Alzheimer's disease, multi infarct dementia, Lewis body dementia, stroke, frontotemporal dementia, progressive supranuclear palsy Huntington's disease, HIV disease, cerebral trauma, drug abuse, mild cognitive disorder, ADHD, Asperger's syndrome, and age-associated memory impairment.

55. The method of claim 51, wherein the disorders usually first diagnosed in infancy, childhood and adolescence are selected from hyperkinetic disorders, deficit/hyperactivity disorder (ADHD), hyperkinetic conduct disorder, attention deficit disorder (ADD), depressive conduct disorder, transient tic disorder, chronic motor or vocal tic disorder, combined vocal and multiple motor tic disorder (de la Tourette), substance induced tic disorders, autistic disorders; excessive masturbation nail-biting, nose-picking and thumb-sucking.

56. The method of claim 51, wherein disorders of psychological development are selected from Asperger's syndrome, Rett's syndrome, autistic disorders, childhood autism, overactive disorder associated with mental retardation and stereotyped movements, specific developmental disorder of motor function, and specific developmental disorders of scholastic skills.

57. The method of claim 51, wherein systemic atrophies primarily affecting the central nervous system are selected from Huntington's disease, multiple sclerosis and amyotrophic lateral sclerosis.

58. The method of claim 51, wherein movement disorders with malfunction or degeneration of basal ganglia are selected from Parkinson's disease, second Parkinsonism, postencephalitic Parkinsonism, Lewis body disease, degenerative diseases of the basal ganglia, tremor, essential tremor and drug-induced tremor, myoclonus, chorea and drug-induced chorea, drug-induced tics and tics of organic origion, drug-induced acute dystonia, drug-induced tardive dyskinesia, L-dopa-induced dyskinesia, restless leg syndrome Stiff-man syndrome, focal dystonia, multiple-focal, segmental dystonia, torsion dystonia, hemispheric, generalised and tardive dystonia, cervical dystonia (torticolli), blepharospasm (cramp of the eyelid), appendicular dystonia, oromandibular dystonia and spasmodic dysphonia, neuroleptic malignant syndrome (NMS), neuroleptic induced parkinsonism, neuroleptic-induced early onset or acute dyskinesia, neuroleptic-induced acute dystonia, neuroleptic-induced acute akathisia, neuroleptic-induced tardive dyskinesia, and neuroleptic-induced tremor.

59. The method of claim 51, wherein behavioural syndromes associated with physiological disturbances and physical factors are selected from nonorganic sleep disorders, nonorganic hypersomnia, nonorganic disorder of the sleep-wake schedule; mental and behavioural disorders associated with the puerperium, postnatal and postpartum depression, eating disorders, anorexia nervosa, and bulimia nervosa.

60. The method of claim 51, wherein disorders of adult personality and behaviour are selected from emotionally unstable, borderline, obsessive-compulsive, anankastic, dependent and passive-aggressive personality disorder; intermittent explosive disorder; pathological gambling; pathological fire-setting (pyromania); pathological stealing (kleptomania); trichotillomania; and Münchausen syndrome.

61. The method of claim 51, wherein schizophrenia and other psychotic disorders disorders are selected from paranoid schizophrenia, hebephrenic schizophrenia, catatonic schizophrenia, undifferentiated schizophrenia, residual schizophrenia, schizophreniform disorders, borderline schizotypal disorder, latent schizotypal disorders, prepsychotic schizotypal disorders, prodromal schizotypal disorders, pseudoneurotic pseudopsychopathic schizophrenia and schizotypal personality disorder, persistent delusional disorders, acute psychotic disorders, transient psychotic disorders, persistent psychotic disorders, induced delusional disorders, manic depressive or mixed type, puerperal psychosis, and nonorganic psychosis.

62. The method of claim 51, wherein mental and behavioural disorders due to psychoactive substance use selected from mental and behavioural disorders due to use of alcohol, opioids, cannabinoids, sedatives or hypnotics, cocaine, mental and behavioural disorders due to the use of stimulants, mental and behavioural disorders due to use of hallucinogens, tobacco, and volatile solvents, mental and behavioural disorders due to multiple drug use and use of psychoactive substances, dependence syndrome, withdrawal state, and withdrawal state with delirium.

63. A method of treating obesity, type II diabetes, metabolic syndrome, glucose intolerance and related health risks, symptoms or disorders in a patient in need thereof comprising administering to said patient a therapeutically effective amount of a compound of claim 1, or a pharmaceutically acceptable salt thereof.

64. A method of treating or preventing disorders associated with enhanced endothelial activity, impaired endothelial barrier or enhanced neoangiogenesis, septic shock; vascular edema, reduced natriuria pathology, inflammatory diseases, asthma, rhinitis, arthritis, rheumatoid diseases, autoimmune diseases, acute renal or liver failure, liver dysfunction, or benign or malignant neoplasia in a patient in need thereof comprising, administering to said patient a therapeutically effective amount of a compound of claim 1, or a pharmaceutically acceptable salt thereof.

65. A method of treating or preventing a disorder associated with thrombosis or embolism in a patient in need thereof comprising, administering to said patient a therapeutically effective amount of a compound of claim 1, or a pharmaceutically acceptable salt thereof.

66. The method of claim 65, wherein said disorder is selected from thrombosis induced tissue infarction in coronary artery disease, in cerebrovascular disease or in peripheral vascular disease; stable and unstable angina; transient ischemic attacks; placenta insufficiency; thrombosis after bypass, angioplasty; thrombosis after stent placement; and thrombosis after heart valve replacement.

67. A pharmaceutical composition or kit, comprising at least one compound of claim 1, or a pharmaceutically acceptable salt thereof, and at least one further pharmaceutically active compound.

68. A method of treating pain or a pain disorder selected from inflammatory pain, hyperalgesia, inflammatory hyperalgesia, migraine, cancer pain, osteoarthritis pain, post-surgical pain, non-inflammatory pain, neuropathic pain, sub-categories of neuropathic pain including peripheral neuropathic pain syndromes, chemotherapy-induced neuropathy, complex regional pain syndrome, HIV sensory neuropathy, neuropathy secondary to tumor infiltration, painful diabetic neuropathy, phantom limb pain, postherpetic neuralgia, postmastectomy pain, trigeminal neuralgia, central neuropathic pain syndromes, central poststroke pain, multiple sclerosis pain, Parkinson disease pain, and spinal cord injury pain in a patient in need thereof, comprising administering to said patient a compound of claim 1, or a pharmaceutically acceptable salt thereof.

69. A method of treating disorders associated with phosphodiesterase 2 or 10 hyperactivity, central nervous system disorders, obesity, type II diabetes, metabolic syndrome, glucose intolerance, disorders associated with thrombosis or embolism, disorders associated with enhanced endothelial activity, impaired endothelial barrier, or enhanced neoangiogenesis, septic shock, vascular edema, reduced natriuria pathology, inflammatory diseases, asthma, rhinitis, arthritis and rheumatoid diseases, autoimmune diseases, acute renal or liver failure, liver dysfunction, or benign or malignant neoplasia in a patient in need thereof comprising, administering to said patient a therapeutically effective amount of a compound of formula (I): 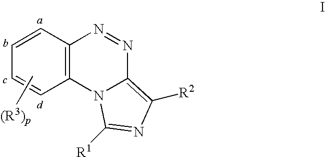
or a pharmaceutically acceptable salt thereof; wherein:
a, b, c, and d indicate four possible positions on the ring for each R³, when present;
p is 0 or an integer from 1 to 4;
R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
any two groups R³ may together be alkylene or alkenylene completing a 3- to 8-membered saturated or unsaturated ring together with the carbon atoms to which they are attached, which ring is unsubstituted or substituted with one or more independently selected Z groups; or
any two groups of R³ may, together with the atoms to which they are attached, form a heterocyclo group which is unsubstituted or substituted with one or more independently selected Z groups;
each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;
each R⁵, R⁶, R⁷, R⁸, R⁹ and R¹⁰ is independently selected hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or
R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or
any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;
each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;
each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more Z¹ groups;
each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;
each Y³ and Y⁴ is independently selected from a single bond, alkylene, alkenylene, and alkynylene; and
each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy.

70. A pharmaceutical composition comprising a compound of formula (I): 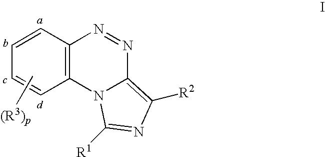
or a pharmaceutically acceptable salt thereof; and a pharmaceutically acceptable carrier; wherein:
a, b, c, and d indicate four possible positions on the ring for each R³, when present;
p is 0 or an integer from 1 to 4;
R¹ is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
R² is selected from hydrogen, R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
each R³ is independently selected from R⁴, —OH, —OR⁴, —SH, —SR⁴, —C(O)H, —C(O)OH, —C(O)R⁴, —C(O)OR⁴, —O—C(O)R⁴, —O—C(O)OR⁴, —SO₃H, —S(O)_{q}R⁴, halo, cyano, nitro, —Y¹—NR⁵R⁶, —Y¹—N(R⁷)—Y²—NR⁸R⁹, —Y¹—N(R¹⁰)—Y²—R⁴, and —P(O)(OR⁴)₂; wherein q is 1 or 2;
any two groups R³ may together be alkylene or alkenylene completing a 3- to 8-membered saturated or unsaturated ring together with the carbon atoms to which they are attached, which ring is unsubstituted or substituted with one or more independently selected Z groups; or
any two groups of R³ may, together with the atoms to which they are attached, form a heterocyclo group which is unsubstituted or substituted with one or more independently selected Z groups;
each R⁴ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups;
each R⁵, R⁶, R⁷, R⁸, R⁹ and R¹⁰ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z groups; or
R⁵ and R⁶ may together be alkylene or alkenylene, completing a 3- to 8-membered saturated or unsaturated ring with the nitrogen atom to which they are attached, which ring is unsubstituted or substituted with one or more independently selected Z groups; or
any two of R⁷, R⁸ and R⁹ may together be alkylene or alkenylene, completing a 3- to 8-membered saturated or unsaturated ring with the nitrogen atom to which they are attached, which ring is unsubstituted or substituted with one or more independently selected Z groups;
each Z group is independently selected from hydrogen, R¹¹, —OH, —OR¹¹, —SH, —SR¹¹, —C(O)H, —C(O)OH, —C(O)R¹¹, —C(O)OR¹¹, —O—C(O)R¹¹, —O—C(O)OR¹¹, —SO₃H, —S(O)_{q}R¹¹, halo, cyano, nitro, —Y¹—NR¹²R¹³, —Y¹—N(R¹⁴)—Y²—NR¹⁵R¹⁶, —Y¹—N(R¹⁷)—Y²—R¹¹, and oxo; wherein q is 1 or 2;
each R¹¹ is independently selected from alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more Z¹ groups;
each R¹², R¹³, R¹⁴, R¹⁵, R¹⁶, and R¹⁷ is independently selected from hydrogen, alkyl, alkenyl, alkynyl, cycloalkyl, cycloalkylalkyl, cycloalkenyl, cycloalkenylalkyl, aryl, aralkyl, heterocyclo, and heterocycloalkyl, each of which is unsubstituted or substituted with one or more independently selected Z¹ groups;
each Y¹ and Y² is independently selected from a single bond, —Y³—S(O)_{q}—Y⁴—, —Y³—C(O)—Y⁴—, —Y³—C(S)—Y⁴—, —Y³—O—Y⁴—, —Y³—S—Y⁴—, —Y³—O—C(O)—Y⁴—, and —Y³—C(O)—O—Y⁴—;
each Y³ and Y⁴ is independently selected from a single bond, alkylene, alkenylene, and alkynylene; and
each Z¹ is independently selected from oxo, halogen, cyano, nitro, hydroxyl, C₁₋₆ alkyl, C₁₋₆ haloalkyl, C₁₋₆ alkoxy, C₁₋₆ haloalkoxy, C₁₋₆ alkylthio, C₁₋₆ alkylsulfinyl, C₁₋₆ alkylsulfonyl, amino, C₁₋₆ alkylamino, di-C₁₋₆-alkylamino, C₁₋₆ alkylcarbonyl, C₁₋₆ alkoxycarbonyl, carboxy, carbamyl, C₁₋₆ alkylcarbamyl, di-C₁₋₆ alkylcarbamyl, C₁₋₆ alkylcarbamyloxy, and di-C₁₋₆-alkylcarbamyloxy.

71. A method of preparing a compound of claim 1, comprising:
(i) reacting an appropriately substituted nitro benzene of formula (1): 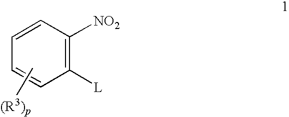
with a substituted imidazole of formula (2): 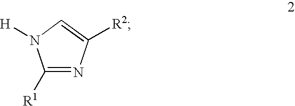
(ii) reducing the nitro group of the product of step (i) to an amino group; and
(iii) reacting the product of step (ii) with a nitrite in the presence of an acid to form the triazine ring structure;
wherein L is a leaving group.

72. The method of claim 71, wherein step (i) is accomplished in the presence of a base.

73. The method of claim 72, wherein said base is selected from a carbonate, hydroxide and amine base.

74. The method of claim 71, wherein L is selected from fluoro, chloro, and bromo.

75. The method of claim 71, wherein the nitro group in (ii) is reduced by catalytic hydrogenation, by use of sodium dithionite, or by use of SnCl₂.

76. The method of claim 71, wherein the acid in (iii) is selected from a mineral acid.

77. The method of claim 76, wherein the acid in (iii) is selected from HCl and H₂SO₄.
